(19) United States
(12) Patent Application Publication
(10) Pub. No.: US 2012/0277233 A1
(43) Pub. Date: Nov. 1, 2012

(54) Pyridyl-Triazine Inhibitors of Hedgehog Signaling

(75) Inventors: Chunlin Tao, Los Angeles, CA (US); Hongna Han, Irvine, CA (US); Xiaowen Sun, Shanghai (CN); Neil Desai, Los Angeles, CA (US)

(73) Assignee: California Capital Equity, LLC, Los Angeles, CA (US)

(21) Appl. No.: 13/377,003

(22) PCT Filed: Jun. 8, 2010
(86) PCT No.: PCT/US10/37717

§ 371 Date: Jul. 3, 2012

Related U.S. Application Data
(60) Provisional application No. 61/185,401, filed on Jun. 9, 2009.

(51) Int. Cl. A61K 31/53; C07D 401/14; C07D 405/14; C07D 413/14; A61P 9/00; C07D 409/14; C07D 417/14; C07D 471/04; C07D 498/04; A61P 35/00; C07D 401/04; A61K 31/5377

(52) U.S. Cl. 514/236.2; 544/212; 514/245; 544/113; 544/209

(57) ABSTRACT

The invention provides pyridyl-triazine derivatives to inhibit the hedgehog signaling pathway and the use of such compounds in the treatment of hyperproliferative diseases and angiogenisis mediated diseases.

### FIELD OF THE INVENTION

The present invention relates generally to the use of pyridyl-triazine derivatives to treat a variety of disorders, diseases and pathologic conditions and more specifically to the use of triazine compounds to inhibit the hedgehog signaling pathway and to the use of compounds to the treatment of hyperproliferative diseases and angiogenesis mediated diseases.

### BACKGROUND OF THE INVENTION

The hedgehog (Hh) gene was first identified during a search for embryonic lethal mutants of Drosophila melanogaster, which found that mutation of Hh resulted in altered segment patterning of the larva (Nusslein-Volhard, C Wieschaus, E. Nature 1980, 287, 795-801). Subsequently the gene was identified in many other invertebrates and vertebrates, including humans. Three mammalian counterparts of the Hh gene, termed Sonic hedgehog (Shh), Dessert hedgehog (Dhh), and Indian hedgehog (Ihh), were identified by combined screening of mouse genomic and cDNA libraries (Echelard, Y.; Epstein, D. J.; et al., Cell 1993, 75, 1417-1430.). Hh undergoes multiple processing events, including autocatalytic cleavage of the C-terminal domain combined with addition of a cholesterol moiety at the cleavage site, and an N-terminal palmitoylation, to generate the active ligand (Lee, J. J.; Ekker, S. C.; et al., Science 1994, 266, 1528-1537; Porter, J. A.; Young, K. E.; et al., Science 1996, 274, 255-259; Pepinsky, R. B.; Zeng, C.; et al., J. Biol. Chem. 1998, 273, 14037-14045).

The receptor of secreted Hh protein is the multipass transmembrane protein Patched (Ptch). Of the two vertebrate homologues of Ptch (Ptch1 and Ptch2), the role of Ptch1 is better understood. In the absence of Hh ligand, Ptch inhibits the activity of the downstream effector Smoothened (Smo). The binding of Hh inactivates Ptch, resulting in activation of Smo (Stone, D. M.; Hynes, M.; et al., Nature 1996, 384, 129-134). In Drosophila, a complex of proteins comprising Fused (Fu), Suppressor of Fused (SuFu), and Costal-2 (Cos2) mediates signaling downstream of Smo and is aided by several kinases, such as protein kinase A (PKA), glycogen synthase kinase 3 (GSK3), and casein kinase 1 (CK1). Mammalian homologues of Fu and Cos2 have not yet been identified, suggesting that the signaling mechanisms differ in mammals and Drosophila. Several mammalian-specific kinases that is required for Shh signaling have been identified (Varjosalo, M.; Bjorklund, M.; et al., Cell 2008, 133, 537-548; Mao, J.; Maye, P.; et al., J. Biol. Chem. 2002, 277, 35156-35161; Riobo, N. A.; Haines, G. M.; et al., Cancer Res. 2006, 66, 839-845). These proteins modulate the function of Gli (Ci in Drosophila), the only transcription factor identified to date that operates directly downstream of Hh.

The first vertebrate Gli gene to be discovered was human Gli1, which was amplified about 50-fold in a malignant glioma (Kinzler, K. W.; Bigner, S. H.; et al., Science 1987, 236, 70-73). Vertebrates have three Gli proteins (Gli1, Gli2, and Gli3), all of which have five highly conserved tandem zinc fingers, a fairly conserved N-terminal domain, several potential PKA sites, and a number of additional small conserved regions in the C-terminal end. Despite these similarities, the functions of the Gli subtypes differ. Both Gli2 and Gli3 contain activation and repressor domains. Consequently, in the absence of upstream Hh signal, full-length Gli3 and, to a lesser extent, Gli2 are constitutively cleaved to generate a truncated repressor form (Dai, P.; Akimaru, H.; et al., J. Biol. Chem. 1999, 274, 8143-8152; Ruiz i Altaba, DeVelopment 1999, 126, 3205-3216; Shin, S. H.; Kogerman, P.; et al., Proc. Natl. Acad. Sci. U.S.A. 1999, 96, 2880-2884). Hh signaling inhibits this cleavage, resulting in full-length Gli2 and Gli3, which have activator function. Gli1, in contrast, does not undergo proteolytic cleavage and acts as a constitutive activator. The transcription of Gli1 gene is initiated by Hh and is also controlled by Gli3.27 Target genes of the Hh pathway other than Gli1 include Ptch, several Wnt and TGF_superfamily proteins, cell cycle proteins such as cyclin D, and stem-cell marker genes such as NANOG and SOX2.30, 31 Investigators are now attempting to comprehensively identify the Gli1-target genes (Yoon, J. W.; Kita, Y.; et al., J. Biol. Chem. 2002, 277, 5548-5555; Yoon, J. W.; Gilbertson, R.; Int. J. Cancer 2008, 124, 109-119).

The Hh signaling pathway is crucial for proper embryonic development (Ingham, P. W.; McMahon, A. P. Genes DeV. 2001, 15, 3059-3087). It is also essential for restraining growth in the nervous system and other tissues and in maintenance of stem cells in adults (Machold, R.; Hayashi, S., et al., Neuron 2003, 39, 937-950; Lavine, K. J.; Kovacs, A.; et al., J. Clin. InVest. 2008, 118, 2404-2414. Balordi, F.; Fishell, G. et al., J. Neurosci. 2007, 27, 14248-14259). The expression and roles of Hh in vertebrate tissues/organs have been extensively described in the recent reviews (Varjosalo, M.; Taipale, J. Genes DeV. 2008, 22, 2454-2472).

Two of the functions of Hh in vertebrate embryonic development are both crucial and relatively well understood: neural tube differentiation and anteroposterior limb patterning. The predominant mechanism of Hh signaling in these functions is paracrine signaling, in which the Hh molecules act in a gradient fashion. For example, in vertebrate limb buds, exposure to different concentrations of Shh modulates patterning of the interdigital mesenchyme, which influences the proper growth of digits in a specific pattern (Tabin, C. J.; McMahon, A. P. Science 2008, 321, 350-352). In neural tube development, Shh produced by the floor plate causes dorsoventral patterning, the specification of ventral cell populations, and general cellular proliferation in the brain.40 Holoprosencephaly, a disorder involving the development of forebrain and midface in which ventral cell types are lost, is caused in humans by mutations that lead to loss of Shh activity (Belloni, E.; Muenke, M.; et al., Nat. Genet. 1996, 14, 353-356).

Another important feature of Shh signaling is that the Gli subtypes have both unique and overlapping functions. While ectopic expression of Gli1 in the midbrain and hindbrain of transgenic mice results in expression of some ventral cell types, mice homozygous for a mutation in the region encoding the zinc finger domain of Gli1 develop normally (Hynes, M.; Stone, D. M.; et al., Neuron, 1997, 19, 15-26; Park, H. L.; Bai, C.; et al., DeVelopment 2000, 127, 1593-1605). However, Gli1/Gli2 double mutant mice have phenotypes with severe multiple defects, including variable loss of the ventral spinal cord, and smaller lungs; therefore, Gli2 plays a more important role in spinal cord and lung development than does Gli1. In contrast, Gli1/Gli3 double mutant mice did not have these phenotypes (Park, H. L.; Bai, C.; et al., DeVelopment 2000, 127, 1593-1605). Gli2 and Gli3 have both been implicated in skeletal development, with each subtype serving specific functional roles. Gli2 mutant mice exhibit severe skeletal abnormalities including cleft palate, tooth defects, absence of vertebral body and intervertebral discs, and shortened limb and sternum (Mo, R.; Freer, A. M.; et al., DeVelopment 1997, 124, 113-123). Gli3 appears to be the major mediator of Shh effect in the limbs, as Gli1/Gli2 double mutant mice had a normal digit number and pattern while Gli3 mutant mice showed polydactyly (Hui, C. C.; Joyner, A. L. Nat. Genet. 1993, 3, 241-246).

Genetic analyses of Gli mutants revealed that the requirement for Gli subtypes development is quite divergent even among vertebrates. In zebrafish, both detour (dtr) mutations (encoding loss-of-function alleles of Gli1) and you-too (yot) mutations (encoding C-terminally truncated Gli2) have defects in body axis formation and expression of Hh-target genes in the brain (Karlstrom, R. O.; Tyurina, O. V.; et al., DeVelopment 2003, 130, 1549-1564), suggesting divergent requirements for Gli1 and Gli2 in mouse and zebrafish.

In adults, the Hh pathway is essential for restraining growth in the nervous system and other tissues and in maintenance of stem cells. Zhang and Kalderon have shown that Hh acts specifically on stem cells in Drosophila ovaries and that these cells cannot proliferate in the absence of Hh (Zhang, Y.; Kalderon, D. Nature 2001, 410, 599-604). Other studies showed that Hh signaling in the postnatal telencephalon both promotes proliferation and maintains populations of neural progenitors, suggesting that Shh signaling in the mammalian telencephalon may participate in the maintenance of a neural stem cell niche. The role of Hh in proliferation of adult neural progenitor cells was confirmed by a study in which Shh was overexpressed and proliferation was inhibited by using a Smo antagonist (Lai, K.; Kaspar, B. K.; et al., Nat. Neurosci. 2003, 6, 21-27).

Hh genes have the ability to induce tissue proliferation. This function is important in embryogenesis and tissue maintenance, but inappropriate activation of the pathway can result in tumorigenesis (Hunter, T. Cell 1997, 88, 333-346). Tumors in about 25% of all cancer deaths are estimated to involve aberrant Hh pathway activation. Tumorigenesis or tumor growth can result from abnormal up-regulation of Hh ligand or from deregulation of the expression or function of downstream components by, for example, loss of Ptch, activating mutations of Smo (Xie, J.; Murone, M.; et al., Nature 1998, 391, 90-92), loss of SuFu, amplification or chromosomal translocation of Gli1 or Gli2 gene amplification or stabilization of Gli2 protein (Bhatia, N.; Thiyagarajan, S.; J. Biol. Chem. 2006, 281, 19320-19326).

The first Hh pathway gene found to be amplified in cancers was Gli1, which was expressed at high levels in human glioblastoma and derived cell lines. Subsequently, Gli1 was found to be consistently expressed in a variety of glial tumors, and Gli1 overexpression was shown to induce central-nerves system hyperproliferation (Dahmane, N.; Sanchez, P.; et al., DeVelopment 2001, 128, 5201-5212). Gli1 overexpression has also been observed in a panel of brain tumors ranging from low-grade to high-grade in a study that identified Gli1 expression as the only reliable marker of Hh pathway activity (Clement, V.; Sanchez, P.; Curr. Biol. 2007, 17, 165-172). Further, cell proliferation in primary cultures of many of these tumors was inhibited by Gli1 small-interfering RNA. Gli1 expression was correlated with tumor grade in PDGF-induced liomagenesis in mice. Hh signaling components other than Gli1 also contribute to tumorigenesis in specific subsets of glioblastomas. In PDGFinduced tumors, expression level of Shh was correlated with the tumor grade. However, other studies found only a subset of gliomas to contain high levels of Shh.

Another cancer with defects in Hh pathway regulation is basal cell carcinoma (BCC). Human Ptch was first identified by virtue of its mutation in patients with Gorlin syndrome (GS), a genetic disease that gives rise to sporadic BCC (Johnson, R. L.; Rothman, A. L.; et al., Science 1996, 272, 1668-1671). The mutations of Ptch identified in BCC include deletions producing truncated proteins and insertion or nonsense mutations accompanied by loss of heterozygosity (LOH) or mutations in the other allele. These mutations inhibit the ability of Ptch to suppress Smo, resulting in constitutive Hh signaling. While Ptch1 abnormalities are detected in the majority of BCC patients, it is now clear that a subset of BCC is also driven by a mutation in Smo that decreases its sensitivity to inhibition by Ptch. In addition, overexpression of Gli1 protein causes BCC-like tumors in mice, establishing the importance of Gli1 transcription in BCC tumorigenesis (Nilsson, M.; Unden, A. B.; et al., Proc. Natl. Acad. Sci. U.S.A. 2000, 97, 3438-3443). The level of Gli1 transcript can be used to discriminate BCC from certain other skin tumors (Hatta, N.; Hirano, T.; et al., J. Cutaneous Pathol. 2005, 32, 131-136). However, blocking of Glibased transcription has not yet been shown to arrest BCC growth.

Medulloblastoma, the most common malignant pediatric brain tumor, is linked with mutations in Ptch and Smo and mutations in other Hh pathway genes such as SuFu and Gli (Pomeroy, S. L.; Tamayo, P.; et al., Nature 2002, 415, 436-442). Inactivation of the Ptch locus by deletion and mutation has been found in about 10% of sporadic medulloblastomas. Shh pathway involvement in these tumors was further confirmed by studies in which treatment of murine medulloblastomas with Smo inhibitors inhibited cell proliferation and reduced tumor growth in mice (Berman, D. M.; Karhadkar, S. S.; et al., Science 2002, 297, 1559-1561; Sanchez, P.; Ruiz i Altaba, Mech. DeV. 2005, 122, 223-230; Romer, J. T.; Kimura, H. et al., Cancer Cell 2004, 6, 229-240). Taylor et al. identified SuFu as a tumorsuppressor gene whose mutation predisposes individuals to medulloblastoma. They found that a subset of children with medulloblastoma carry germline and somatic mutations in SuFu, accompanied by loss of heterozygosity of the wild-type allele. Several of these mutations encoded truncated SuFu proteins that are unable to export Gli protein from the nuclei. In addition, the tumor-suppressor REN has also been linked with medulloblastoma in which the allelic deletion and reduced expression of REN are frequently observed. It is suggested that it inhibits medulloblastoma growth by negatively regulating the Hh pathway (C.; Zazzeroni, F.; Gallo, R.; et al., Proc. Natl. Acad. Sci. U.S.A. 2004, 101, 10833-10838; Argenti, B.; Gallo, R.; et al., J. Neurosci. 2005, 25, 8338-8346).

Hh has also been shown to be an early and late mediator of pancreatic cancer tumorigenesis. Shh was not detected in normal adult human pancreata but was aberrantly expressed in 70% of pancreatic adenocarcinoma specimens (Thayer, S. P.; di Magliano, M. P.; et al., Nature 2003, 425, 851-856). Participation of Shh signaling has been indicated at multiple stages of pancreatic carcinogenesis and is accompanied by multiple oncogenic factors, including K-Ras, one of the most frequently mutated genes in pancreatic cancer (Morton, J. P.; Mongeau, M. E.; et al., Proc. Natl. Acad. Sci. U.S.A. 2007, 104, 5103-5108; Ji, Z.; Mei, F. C.; et al., J. Biol. Chem. 2007, 282, 14048-14055). Activated Hh signaling was detected in cell lines established from primary and metastatic pancreatic adenocarcinomas, and the Smo inhibitor cyclopamine induced apoptosis in a subset of the pancreatic cancer cell lines both in culture and in mice (Sheng, T.; Li, C.; et al., Mol. Cancer. 2004, 3, 29).

Numerous studies indicate that Hh signaling is involved in prostate cancer. Sanchez and others reported the expression of Shh-Gli pathway components in adult human prostate cancer. Treatment of primary prostate tumor cultures and metastatic prostate cancer cell lines with Smo inhibitors blocked the pathway and proliferation. Increased expression of Shh in prostate cancer cells up-regulates Gli1 expression and dramatically accelerates the growth of prostate tumor xenografts (Fan, L.; Pepicelli, C. V.; et al., Endocrinology 2004, 145, 3961-3970). Elevated Shh activity distinguished metastatic from localized prostate cancer, and manipulation of this pathway modulated the invasiveness and metastasis of these tumors (Karhadkar, S. S.; Bova, G. S.; et al., Nature 2004, 431, 707-712).

Hh signaling has also been implicated in various other cancers, such as lung, colorectal, bladder, endometrial, ovarian, and esophageal carcinomas and rhabdomyosarcoma (Chi, S.; Huang, S.; et al., Cancer Lett. 2006, 244, 53-60; Watkins, D. N.; Berman, D. M.; et al., Nature 2003, 422, 313-317; Qualtrough, D.; Buda, A.; et al., Int. J. Cancer 2004, 110, 831-837; McGarvey, T. W.; Maruta, Y.; Oncogene 1998, 17, 1167-1172; Feng, Y. Z.; Shiozawa, T.; et al., Clin. Cancer Res. 2007, 13, 1389-1398; Bhattacharya, R.; Kwon, J.; et al., Clin. Cancer Res. 2008, 14, 7659-7666; Mori, Y.; Okumura, T.; et al., Oncology 2006, 70, 378-389; Tostar, U.; Maim, C. J.; et al., J. Pathol. 2006, 208, 17-25; Hahn, H.; Wojnowski, L.; et al., Nat. Med. 1998, 4, 619-622). The role of Hh-Gli signaling pathway in cancer and its potential as therapeutic target have been reviewed in more detail in recent articles.

The aberrant activation of Hh-Gli signaling in several cancers has made it an attractive target for anticancer drug discovery. Various inhibitors of hedgehog signaling have been investigated such as Cyclopamine, a natural alkaloid that had been showed to arrest cell cycle at arrest cell cycle at G0-G1 and to induce apoptosis in SCLC. Cyclopamine is believed to inhibit Smo by binding to its heptahelical bundle. Currently it is in preclinical and clinical studies as an anticancer agent (Kolterud, Å.; Toftga°rd, R. Drug DiscoVery Today: Ther. Strategies 2007, 4, 229-235). A number of Smo inhibitors have now been reported and can be classified as cyclopamine analogues or synthetic Smo antagonists. Several pharmaceutical companies have identified new Smo inhibitors with druglike properties by optimization of highthroughput screen hits. One such small molecule, GDC-0449, was developed by Curis and Genentech, is currently in phase I/II clinical trials for advanced BCC and solid epithelial tumor (Gunzner, J.; Sutherlin, D.; et al., WO2006028958, Mar. 16, 2006). Despite with these compounds, there still remains a need for potent inhibitors of the hedgehog signaling pathway.

### DETAILED DESCRIPTION OF THE INVENTION

The present invention is related to compounds showed as in Formula (I)

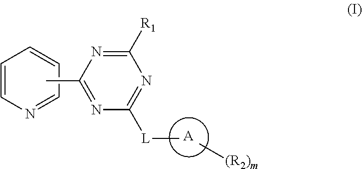

Or a pharmaceutically acceptable salt thereof, wherein:

### L is NR₃CO, NR₃SO₂, NR₃CONH, NR₃CSNH or NR₃CHR₄;

R₁ is selected from:
(i) amino, alkyl amino, aryl amino, heteroaryl amino;
(ii) Alkylthio, sulfinyl, sulfonyl, sulfamoyl;
(iii) Alkyloxy, Alkanoyl, alkoxycarbonyl;
(iv) Hydrogen, C₁-C₆ alkyl, cycloalkyl, C₂-C₆ alkenyl, C₂-C₆ alkynyl;
(v) aryl, heterocyclic, heteroaryl;
(vi) C₁-C₆ trifluoroalkyl, cyano and
(vii) groups of the formula (a):

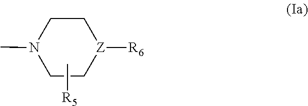

wherein:
R₅ represents hydrogen, C₁-C₄ alkyl, oxo;
Z is CH, when R₆ is hydrogen; or Z—R₆ is O; or Z is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁-C₆alkyl)aminocarbonyl, each of which is substituted with from 0 to 4 substituents independently chosen from halogen, hydroxy, cyano, amino, —COOH and oxo;
Ring A is aryl, heterocycle, heteroaryl;
R₂ is hydroxyl, halogen, amino, nitro, cyano, alkyl, alkenyl, alkynyl, Alkanoyl, Alkylthio, sulfonyl, sulfinyl, alkoxy, alkoxycarbonyl, carbamoyl, acylamine, sulfamoyl or sulfonamide;
or R₂ is a aryl, heterocycle or heteroaryl that is optionally substituted with hydroxyl, halogen, amino, nitro, cyano, alkyl, acyl, sulfonyl, sulfinyl, alkoxy, carbamoyl, acylamine, sulfamoyl and sulfonamide.
R₃ and R₄ are independently selected from hydrogen or an optionally substituted C1-4 alkyl group;
m is 0-4.

In a particular embodiment, compounds of the invention have the general formular Ia.

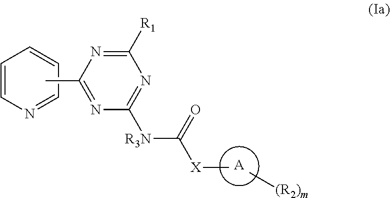

Wherein A, R₁, R₂, R₃, R₄ and m are as defined herein and

X is absent, O, CR₄R₇ or NR₃
R₇ is hydrogen or an optionally substituted C1-4 alkyl group;
In another particular embodiment, compounds of the invention have the general formular Ib.

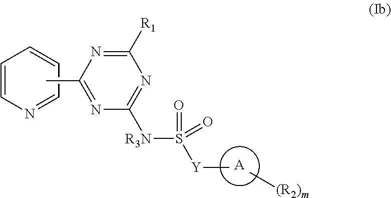

Wherein A, R₁, R₂, R₃, m are as defined herein and

Y is absent or CR₄R₇
R₄, R₇ are as defined herein.

The present invention also relates to compounds as shown in Formula (A):

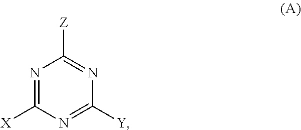

or a pharmaceutically acceptable salt thereof, wherein:
Y is selected from -K-A¹-R¹;
K is selected from NR³C(O) and NR⁴C(O)NR⁵;
A¹ is selected from aryl, heteroaryl, and heterocyclyl;
R¹ is one or more substituents independently selected from H, halo, nitro, C₁-C₆ alkylsulfonyl, —OR⁴, C₁-C₆ alkyl, and C₁-C₆ haloalkyl;
R³ is selected from H, C₁-C₆ alkyl, and —C(O)-A¹-R¹;
R⁴ and R⁵ are each independently selected from H and C₁-C₆ alkyl;
X is pyridinyl;
Z is selected from H, C₁-C₆ alkyl, C₁-C₆ alkylthio, C₁-C₆ alkoxy, C₁-C₆ haloalkyl, —NR⁴R⁵, and cyano.

The present invention also relates to compounds as shown in Formula (A):

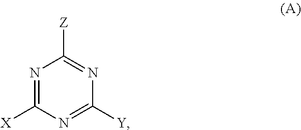

or a pharmaceutically acceptable salt thereof, wherein:
Y is -K-A¹-R¹;
K is selected from NR³C(O) and NR⁴C(O)NR⁵;
A¹ is selected from phenyl and furanyl;
R¹ is one or more substituents independently selected from H, halo, nitro, C₁-C₆ alkylsulfonyl, —OR⁴, C₁-C₆ alkyl, and C₁-C₆ haloalkyl;
R³ is selected from H, C₁-C₆ alkyl, and —C(O)-A¹-R¹;
R⁴ and R⁵ are each independently selected from H and C₁-C₆ alkyl;
X is pyridinyl;
Z is selected from C₁-C₆ alkyl, C₁-C₆ alkylthio, and —NR⁴R⁵.

The following definitions refer to the various terms used above and throughout the disclosure.

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that include, variables (e.g. X, Ar.). Unless otherwise specified, each variable within such a formula is defined independently of any other variable, and any variable that occurs more than one time in a formula is defined independently at each occurrence.

The term “halo” or “halogen” refers to fluorine, chlorine, bromine or iodine.

The term “alkyl” herein alone or as part of another group refers to a monovalent alkane (hydrocarbon) derived radical containing from 1 to 12 carbon atoms unless otherwise defined. Alkyl groups may be substituted at any available point of attachment. An alkyl group substituted with another alkyl group is also referred to as a “branched alkyl group”. Exemplary alkyl groups include methyl, ethyl, propyl, isopropyl, n-butyl, t-butyl, isobutyl, pentyl, hexyl, isohexyl, heptyl, dimethylpentyl, octyl, 2,2,4-trimethylpentyl, nonyl, decyl, undecyl, dodecyl, and the like. Exemplary substituents include but are not limited to one or more of the following groups: alkyl, aryl, halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). In some preferred embodiments of the present invention, alkyl groups are substituted with, for example, amino, heterocycloalkyl, such as morpholine, piperazine, piperidine, azetidine, hydroxyl, methoxy, or heteroaryl groups such as pyrrolidine,

The term ‘cycloalkyl” herein alone or as part of another group refers to fully saturated and partially unsaturated hydrocarbon rings of 3 to 9, preferably 3 to 7 carbon atoms. The examples include cyclopropyl, cyclobutyl, cyclopentyl and cyclohexyl, and like. Further, a cycloalkyl may be substituted. A substituted cycloalkyl refers to such rings having one, two, or three substituents, selected from the group consisting of halo, alkyl, substituted alkyl, alkenyl, alkynyl, nitro, cyano, oxo (═O), hydroxy, alkoxy, thioalkyl, —CO₂H, —C(═O)H, CO₂-alkyl, —C(═O)alkyl, keto, ═N—OH, ═N—O-alkyl, aryl, heteroaryl, heterocyclo, —NR′R″, —C(═O)NR′R″, —CO₂NR′R″, —C(═O)NR′R″, —NR′CO₂R″, —NR′C(═O)R″, —SO₂NR′R″, and —NR′SO₂R″, wherein each of R′ and R″ are independently selected from hydrogen, alkyl, substituted alkyl, and cycloalkyl, or R′ and R″ together form a heterocyclo or heteroaryl ring.

The term ‘alkenyl” herein alone or as part of another group refers to a hydrocarbon radical straight, branched or cyclic containing from 2 to 12 carbon atoms and at least one carbon to carbon double bond. Examples of such groups include the vinyl, allyl, 1-propenyl, isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, and like. Alkenyl groups may also be substituted at any available point of attachment. Exemplary substituents for alkenyl groups include those listed above for alkyl groups, and especially include C3 to C7 cycloalkyl groups such as cyclopropyl, cyclopentyl and cyclohexyl, which may be further substituted with, for example, amino, oxo, hydroxyl, etc.

The term “alkynyl” refers to straight or branched chain alkyne groups, which have one or more unsaturated carbon-carbon bonds, at least one of which is a triple bond. Alkynyl groups include C2-C8 alkynyl, C2-C6 alkynyl and C2-C4 alkynyl groups, which have from 2 to 8, 2 to 6 or 2 to 4 carbon atoms, respectively. Illustrative of the alkynyl group include ethenyl, propenyl, isopropenyl, butenyl, isobutenyl, pentenyl, and hexenyl. Alkynyl groups may also be substituted at any available point of attachment. Exemplary substituents for alkynyl groups include those listed above for alkyl groups such as amino, alkylamino, etc. The numbers in the subscript after the symbol “C” define the number of carbon atoms a particular group can contain.

The term “alkoxy” alone or as part of another group denotes an alkyl group as described above bonded through an oxygen linkage (—O—). Preferred alkoxy groups have from 1 to 8 carbon atoms. Examples of such groups include the methoxy, ethoxy, n-propoxy, isopropoxy, n-butoxy, isobutoxy, sec-butoxy, tert-butoxy, n-pentyloxy, isopentyloxy, n-hexyloxy, cyclohexyloxy, n-heptyloxy, n-octyloxy and 2-ethylhexyloxy.

The term “alkylthio” refers to an alkyl group as described above attached via a sulfur bridge. Preferred alkoxy and alkylthio groups are those in which an alkyl group is attached via the heteroatom bridge. Preferred alkylthio groups have from 1 to 8 carbon atoms. Examples of such groups include the methylthio, ethylthio, n-propythiol, n-butylthiol, and like.

The term “oxo,” as used herein, refers to a keto (C═O) group. An oxo group that is a substituent of a nonaromatic carbon atom results in a conversion of —CH₂— to —C(═O)—.

The term “alkanoyl” refers to groups of the formula: —C(O)R, where the R group is a straight or branched C1-C6 alkyl group, cycloalkyl, aryl, or heteroaryl.

The term “alkoxycarbonyl” herein alone or as part of another group denotes an alkoxy group bonded through a carbonyl group. An alkoxycarbonyl radical is represented by the formula: —C(O)OR, where the R group is a straight or branched C1-C6 alkyl group, cycloalkyl, aryl, or heteroaryl.

The term “aryl” herein alone or as part of another group refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 20 atoms therein, with alternating (resonating) double bonds between adjacent carbon atoms or suitable heteroatoms. Aryl groups may optionally be substituted with one or more groups including, but not limited to halogen such as I, Br, F, or Cl; alkyl, such as methyl, ethyl, propyl, alkoxy, such as methoxy or ethoxy, hydroxy, carboxy, carbamoyl, alkyloxycarbonyl, nitro, alkenyloxy, trifluoromethyl, amino, cycloalkyl, aryl, heteroaryl, cyano, alkyl S(O)ₘ (m=O, 1, 2), or thiol.

The term “amino” herein alone or as part of another group refers to —NH₂, an “amino” may optionally be substituted with one or two substituents, which may be the same or different, such as alkyl, aryl, arylalkyl, alkenyl, alkynyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl, thioalkyl, carbonyl or carboxyl. These substituents may be further substituted with a carboxylic acid, any of the alkyl or aryl substituents set out herein. In some embodiments, the amino groups are substituted with carboxyl or carbonyl to form N-acyl or N-carbamoyl derivatives.

The term “alkylsulfonyl” refers to groups of the formula (SO₂)-alkyl, in which the sulfur atom is the point of attachment. Preferably, alkylsulfonyl groups include C1-C6 alkylsulfonyl groups, which have from 1 to 6 carbon atoms. Methylsulfonyl is one representative alkylsulfonyl group.

The term “heteroatom” refers to any atom other than carbon, for example, N, O, or S.

The term “heteroaryl” herein alone or as part of another group refers to substituted and unsubstituted aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom.

The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero, one, two or three substituents selected from the group consisting of halo, alkyl, substituted alkyl, alkenyl, alkynyl, aryl, nitro, cyano, hydroxy, alkoxy, thioalkyl, —CO₂H, —C(═O)H, —CO₂-alkyl, —C(═O)alkyl, phenyl, benzyl, phenylethyl, phenyloxy, phenylthio, cycloalkyl, substituted cycloalkyl, heterocyclo, heteroaryl, —NR′R″, —C(═O)NR′R″, —CO₂NR′R″, —C(═O)NR′R″, —NR′CO₂R″, —NR′C(═O)R″, —SO₂NR′R″, and —NR′SO₂R″, wherein each of R′ and R″ is independently selected from hydrogen, alkyl, substituted alkyl, and cycloalkyl, or R′ and R″ together form a heterocyclo or heteroaryl ring.

Preferably monocyclic heteroaryl groups include pyrrolyl, pyrazolyl, pyrazolinyl, imidazolyl, oxazolyl, diazolyl, isoxazolyl, thiazolyl, thiadiazolyl, S isothiazolyl, furanyl, thienyl, oxadiazolyl, pyridyl, pyrazinyl, pyrimidinyl, pyridazinyl, triazinyl and the like.

Preferably bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, dihydroisoindolyl, tetrahydroquinolinyl and the like.

Preferably tricyclic heteroaryl groups include carbazolyl, benzidolyl, phenanthrollinyl, acridinyl, phenanthridinyl, xanthenyl and the like.

The term “heterocycle” or “heterocycloalkyl” herein alone or as part of another group refers to a cycloalkyl group (nonaromatic) in which one of the carbon atoms in the ring is replaced by a heteroatom selected from O, S or N. The “heterocycle” has from 1 to 3 fused, pendant or spiro rings, at least one of which is a heterocyclic ring (i.e., one or more ring atoms is a heteroatom, with the remaining ring atoms being carbon). The heterocyclic ring may be optionally substituted which means that the heterocyclic ring may be substituted at one or more substitutable ring positions by one or more groups independently selected from alkyl (preferably lower alkyl), heterocycloalkyl, heteroaryl, alkoxy (preferably lower alkoxy), nitro, monoalkylamino (preferably a lower alkylamino), dialkylamino (preferably a alkylamino), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy; lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. A heterocyclic group may generally be linked via any ring or substituent atom, provided that a stable compound results. N-linked heterocyclic groups are linked via a component nitrogen atom.

Typically, a heterocyclic ring comprises 1-4 heteroatoms; within certain embodiments each heterocyclic ring has 1 or 2 heteroatoms per ring. Each heterocyclic ring generally contains from 3 to 8 ring members (rings having from to 7 ring members are recited in certain embodiments), and heterocycles comprising fused, pendant or spiro rings typically contain from 9 to 14 ring members which consists of carbon atoms and contains one, two, or three heteroatoms selected from nitrogen, oxygen and/or sulfur.

Examples of “heterocycle” or “heterocycloalkyl groups include piperazine, piperidine, morpholine, thiomorpholine, pyrrolidine, imidazolidine and thiazolide.

The term “carbamoyl” herein refers to aminocarbonyl containing substituent represented by the formular C(O)N(R)₂ in which R is H, hydroxyl, alkyl, a carbocycle, a heterocycle, carbocycle-substituted alkyl or alkoxy, or heterocycle-substituted alkyl or alkoxy wherein the alkyl, alkoxy, carbocycle and heterocycles are as herein defined. Carbomoyl groups include alkylaminocarbonyl (e.g. ethylaminocarbonyl, Et-NH—CO—), arylaminocarbonyl (e.g. phenylaminocarbonyl), aralkylaminocrbonyl (e.g. benzoylaminocarbonyl), heterocycleaminocarbonyl (e.g. piperizinylaminocarbonyl), and in particular a heteroarylaminocarbonyl (e.g. pyridylaminocarbonyl).

The term “sulfamoyl” herein refers to —SO₂—N(R)₂ wherein each R is independently H, alkyl, carbocycle, heterocycle, carbocycloalkyl or heterocycloalkyl. Particular sulfamoyl groups are alkylsulfamoyl, for example methylsulfamoyl (—SO₂—NHMe); arylsulfamoyl, for example phenylsulfamoyl; aralkylsulfamoyl, for example benzylsulfamoyl.

The term “sulfinyl” herein refers to —SOR wherein R is alkyl, carbocycle, heterocycle, carbocycloalkyl or heterocycloalkyl. Particular sulfinyl groups are alkylsulfinyl (i.e. —SO-alkyl), for example methylsulfinyl; arylsulfinyl (i.e. —SO-aryl) for example phenylsulfinyl; arakylsulfinyl, for example benzylsulfinyl.

The term “sulfoamide” herein refers to —NR—SO₂—R wherein each R is independently H, alkyl, carbocycle, heterocycle, carbocycloalkyl or heterocycloalkyl), a carbocycle or a heterocycle. Particular sulfonamide groups are alkylsulfonamide (e.g. —NH—SO₂-alkyl), for example methylsulfonamide; arylsulfonamide (e.g. —NH—SO₂-aryl), for example phenylsulfonamide; aralkylsulfonamide, for example benzylsulfonamide.

The term “sulfonyl” herein refers to —SO₂—R group wherein R is alkyl, carbocycle, heterocycle, carbocycloalkyl or heterocycloalkyl. Particular sulfonyl groups are alkylsulfonyl (e.G. —SO₂-alkyl), for example methylsulfonyl; arylsulfonyl, for example phenylsulfonyl; araalkylsulfonyl, for example benzylsulfonyl.

A dash (“-”) that is not between two letters or symbols is used to indicate a point of t attachment for a substituent. For example, —CONH₂ is attached through the carbon atom.

The term “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member.

The term “optionally substituted” as it refers that the aryl or heterocyclyl or other group may be substituted at one or more substitutable positions by one or more groups independently selected from alkyl (preferably lower alkyl), alkoxy (preferably lower alkoxy), nitro, monoalkylamino (preferably with one to six carbons), dialkylamino (preferably with one to six carbons), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy and lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. Optional substitution is also indicated by the phrase “substituted with from 0 to X substituents,” where X is the maximum number of possible substituents. Certain optionally substituted groups are substituted with from 0 to 2, 3 or 4 independently selected substituents.

The term “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC— (CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred. It will be apparent that each compound of Formula I may, but need not, be formulated as a hydrate, solvate or non-covalent complex. In addition, the various crystal forms and polymorphs are within the scope of the present invention. Also provided herein are prodrugs of the compounds of Formula I.

Groups that are “optionally substituted” are unsubstituted or are substituted by other than hydrogen at one or more available positions. Such optional substituents include, for example, hydroxy, halogen, cyano, nitro, C1-C6 alkyl, C2-C6 alkenyl, C2-C6 alkynyl, C1-C6 alkoxy, C2-C6 alkyl ether, C3-C6 alkanone, C2-C6 alkylthio, amino, mono- or di-(C1-C6 alkyl)amino, C1-C6 haloalkyl, —COOH, —CONH₂, mono- or di-(C1-C6 alkyl)aminocarbonyl, —SO₂NH₂, and/or mono or di(C1-C6 alkyl) sulfonamido, as well as carbocyclic and heterocyclic groups.

Optional substitution is also indicated by the phrase “substituted with from 0 to X substituents,” where X is the maximum number of possible substituents. Certain optionally substituted groups are substituted with from 0 to 2, 3 or 4 independently selected substituents.

In a particular embodiment A is a ring selected from the below groups:

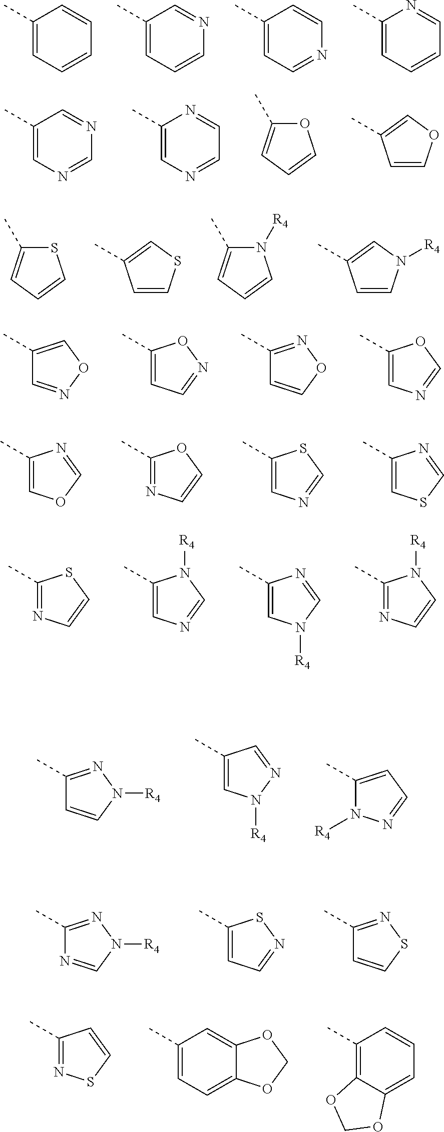

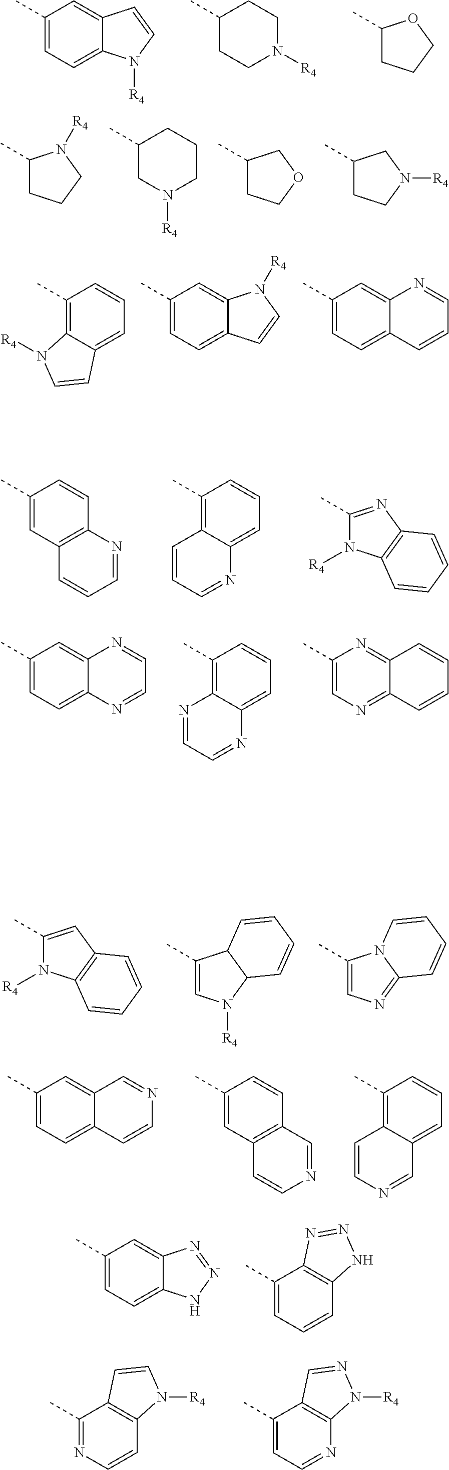

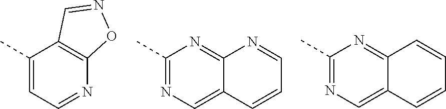

Preferred R₁ groups of formula (I) are listed below:

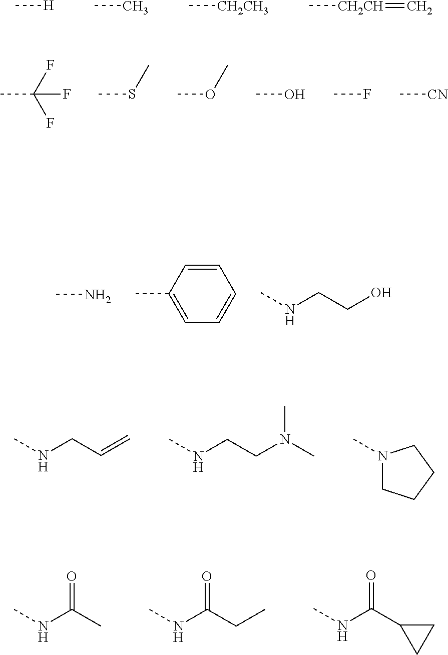

Examples of specific compounds of the present invention are those compounds defined in the following:

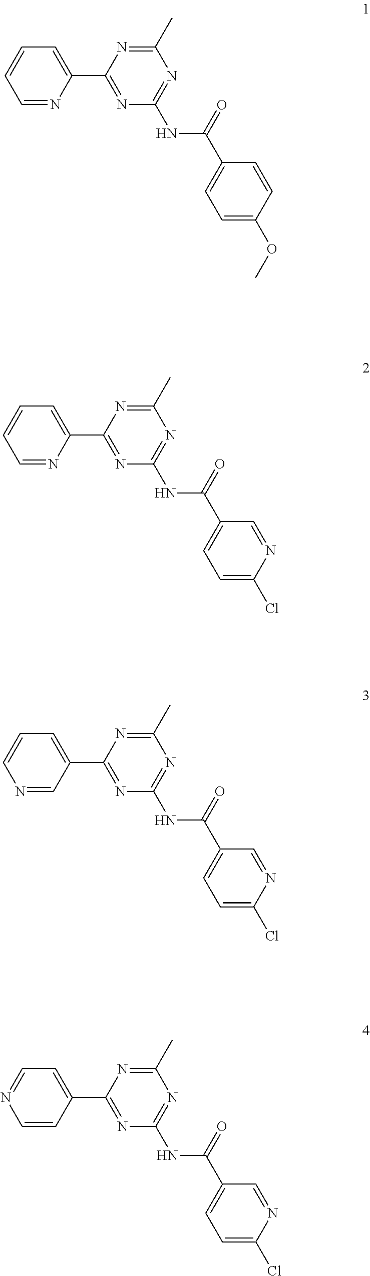

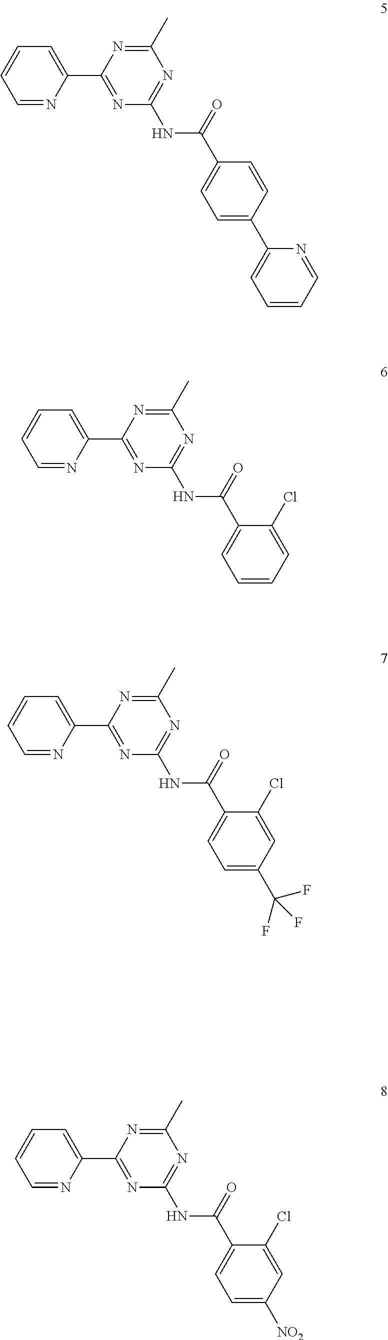

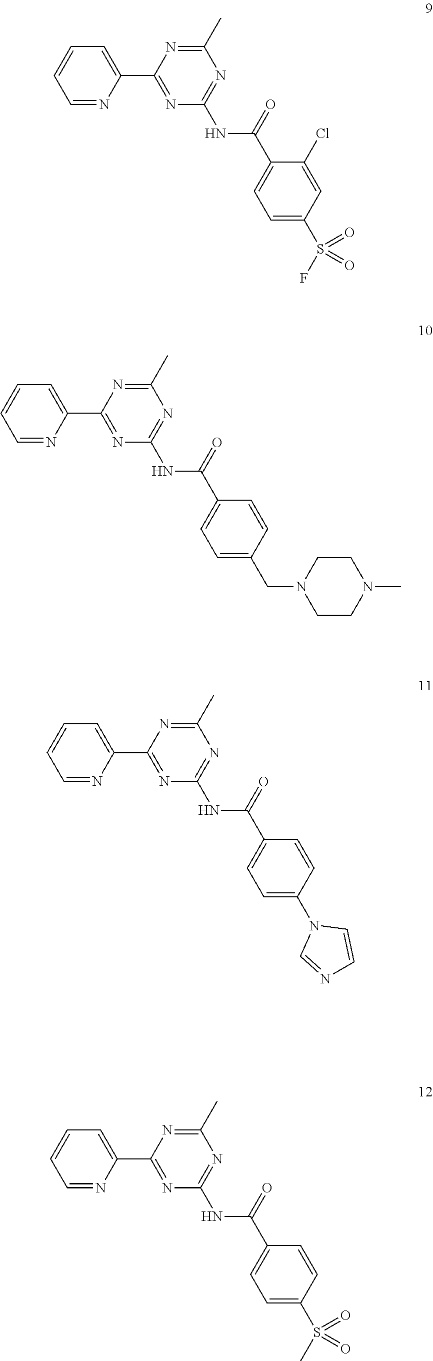

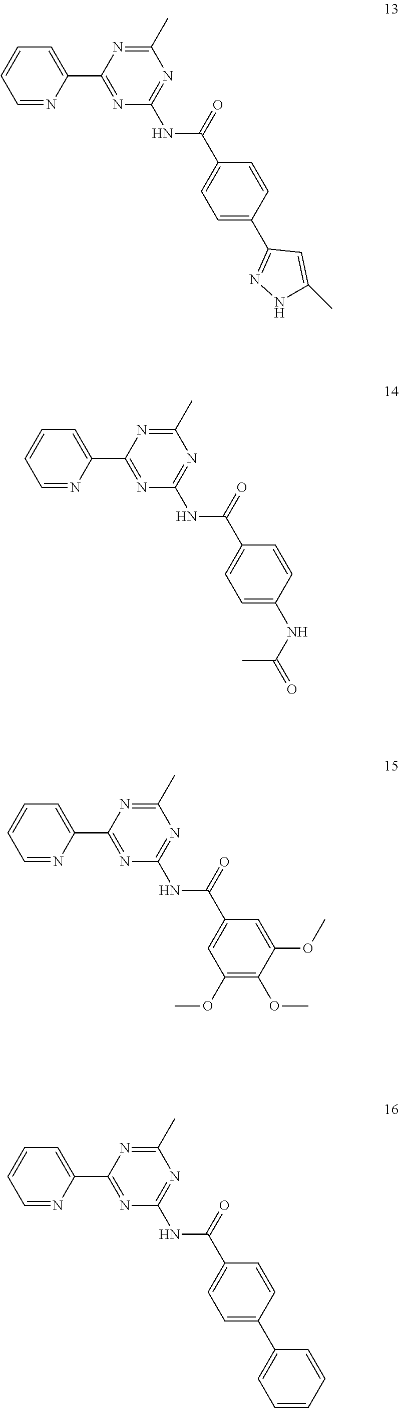

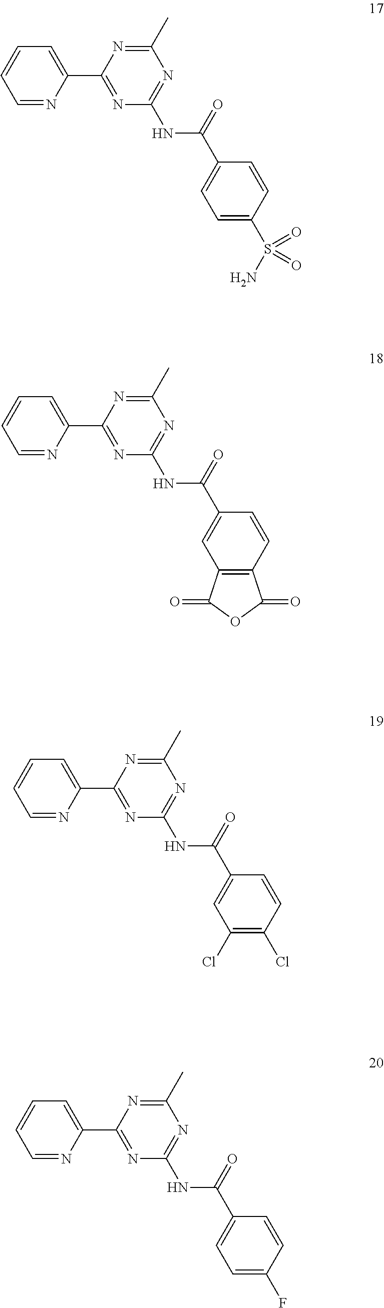

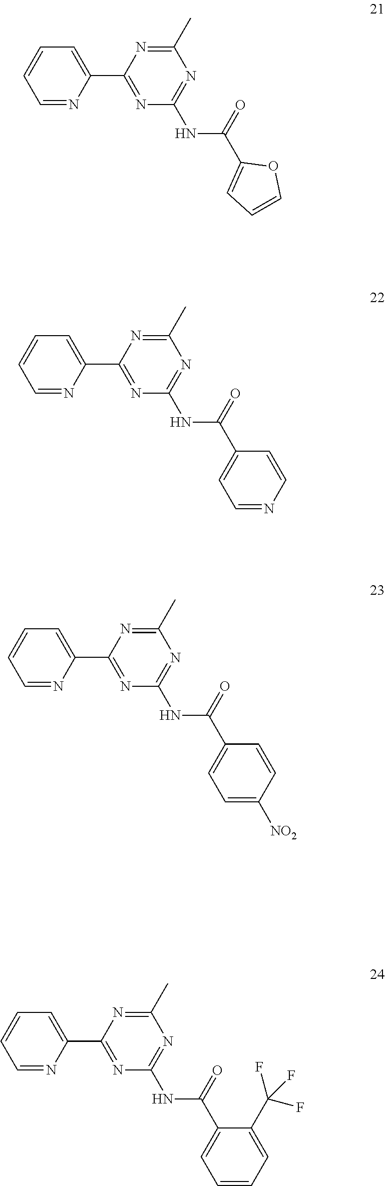

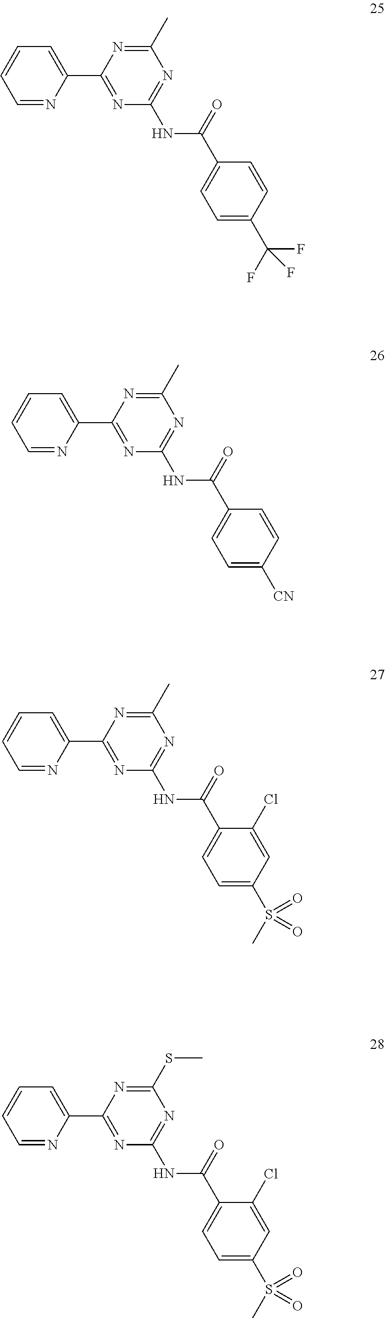

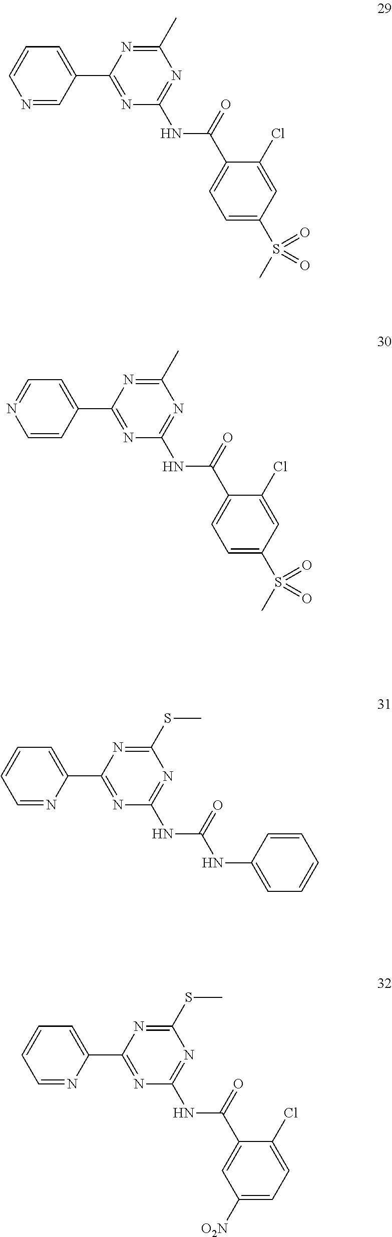

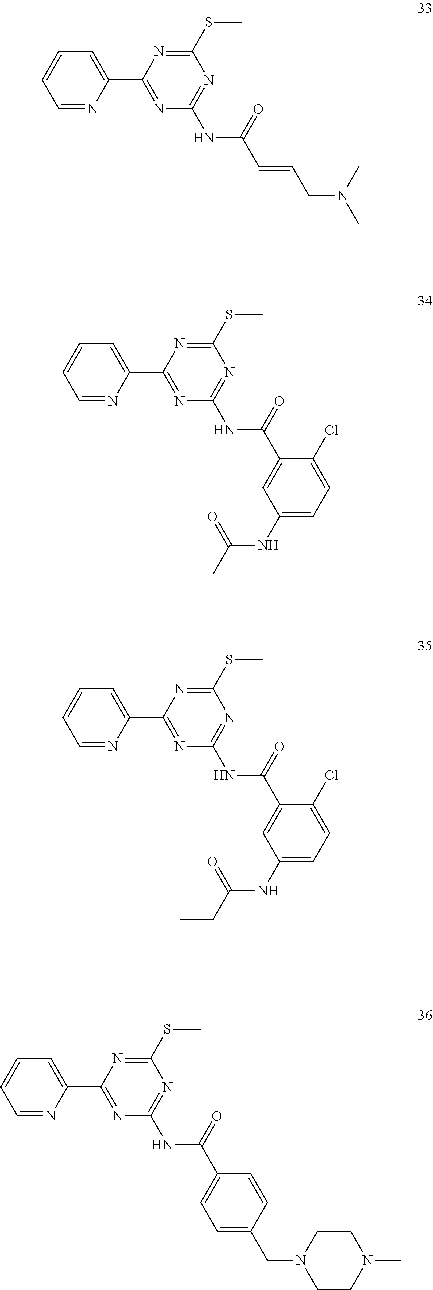

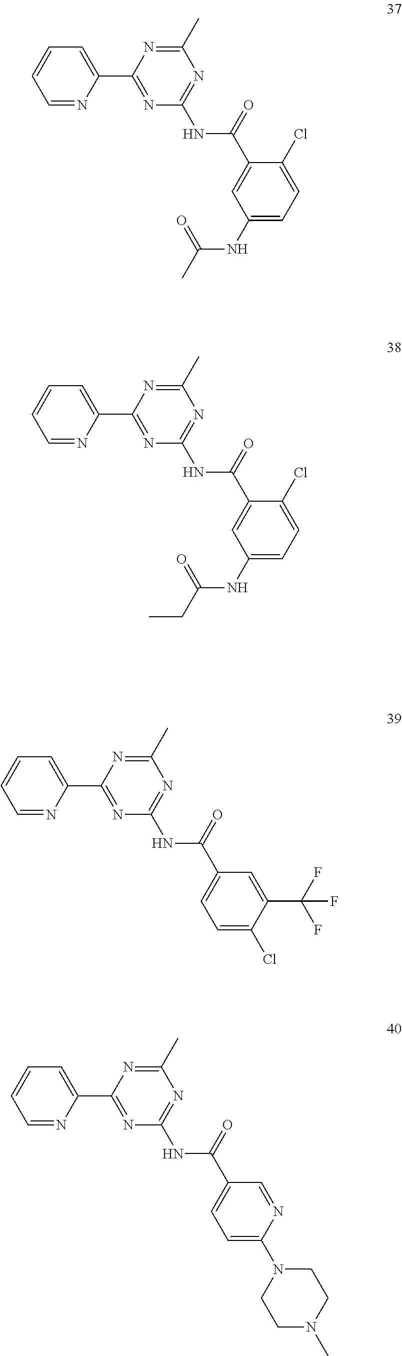

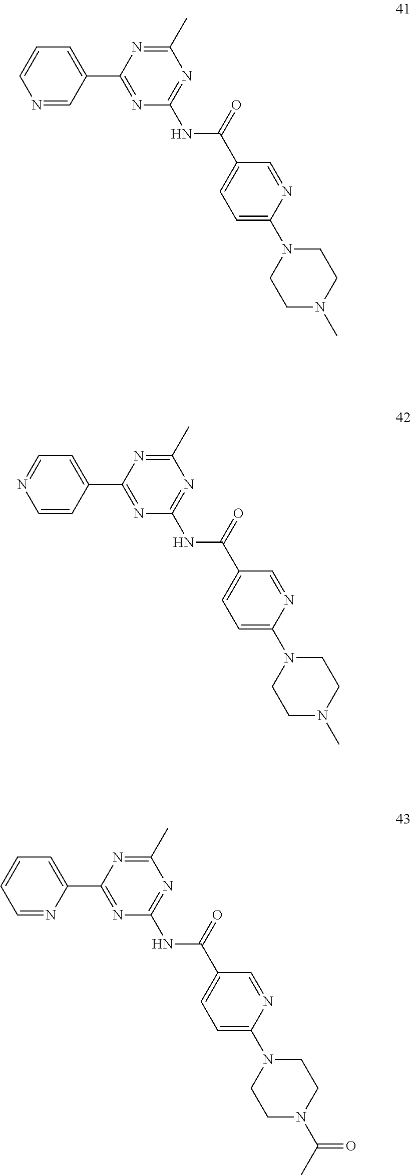

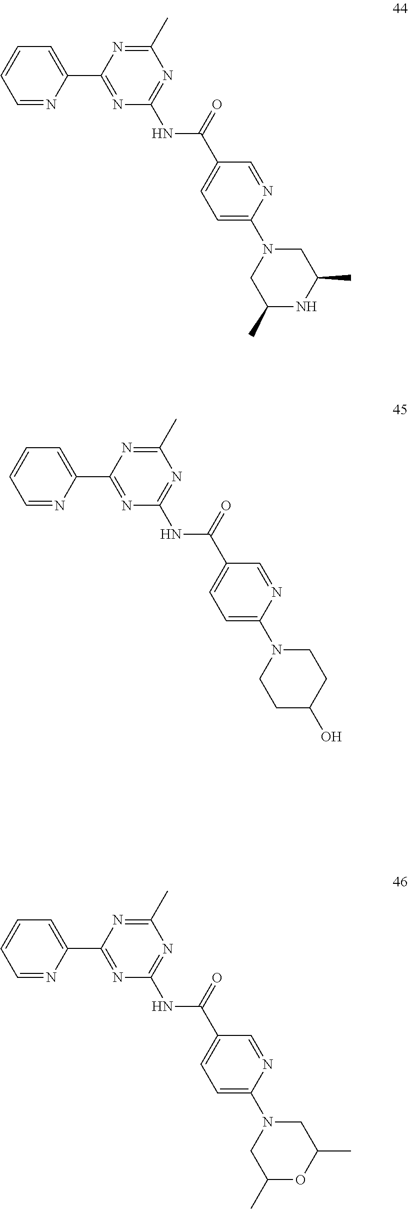

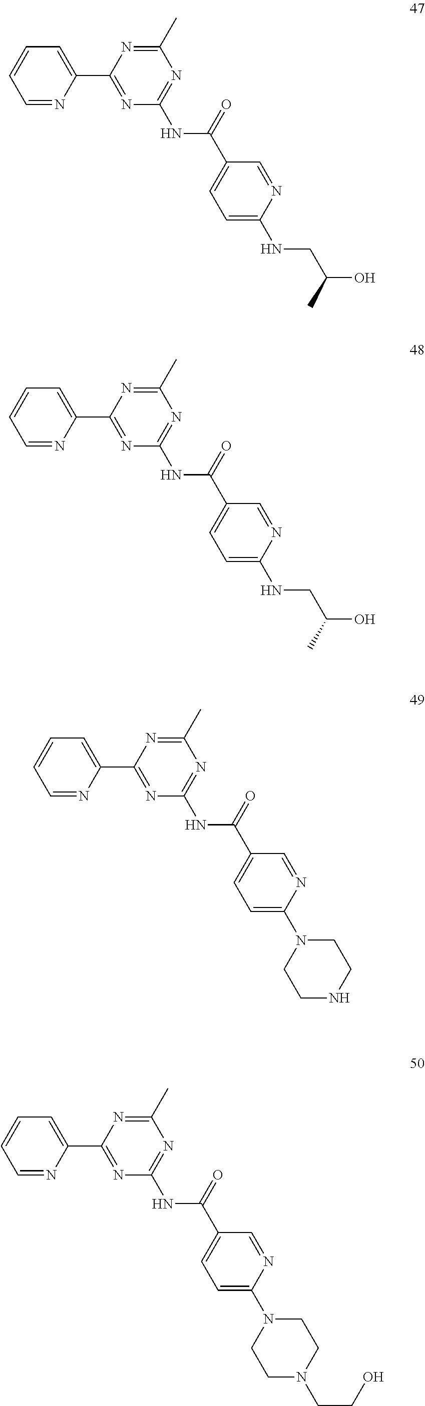

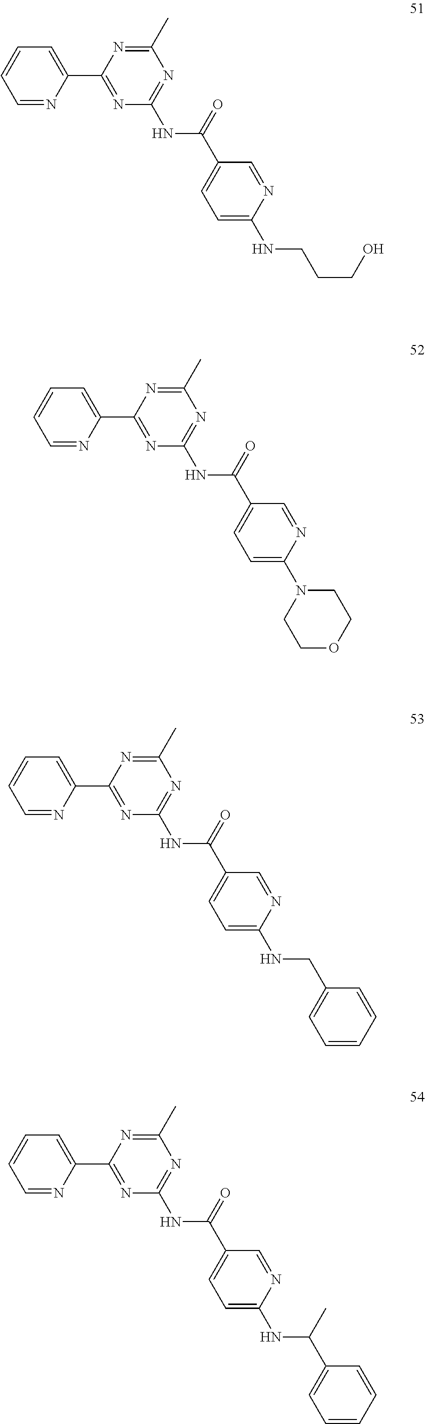

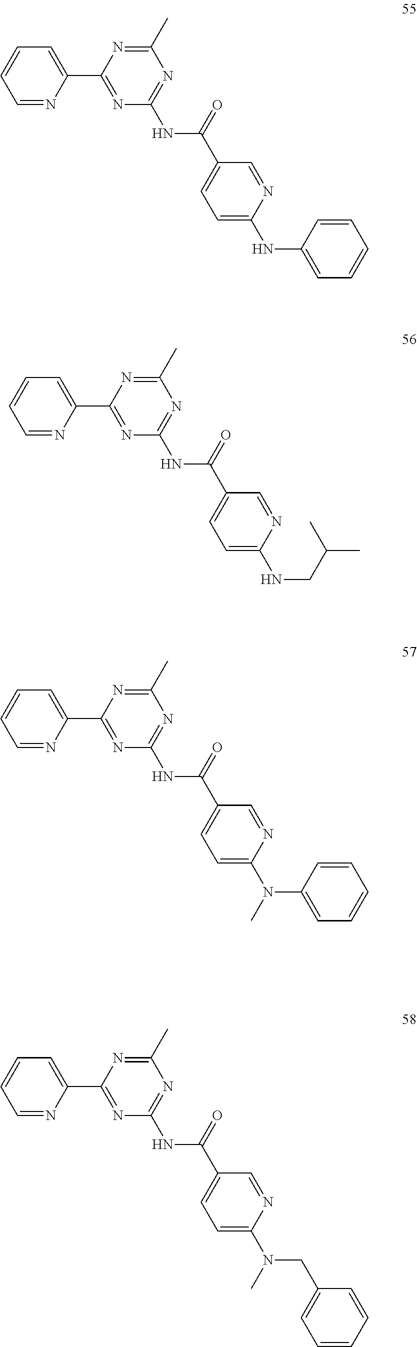

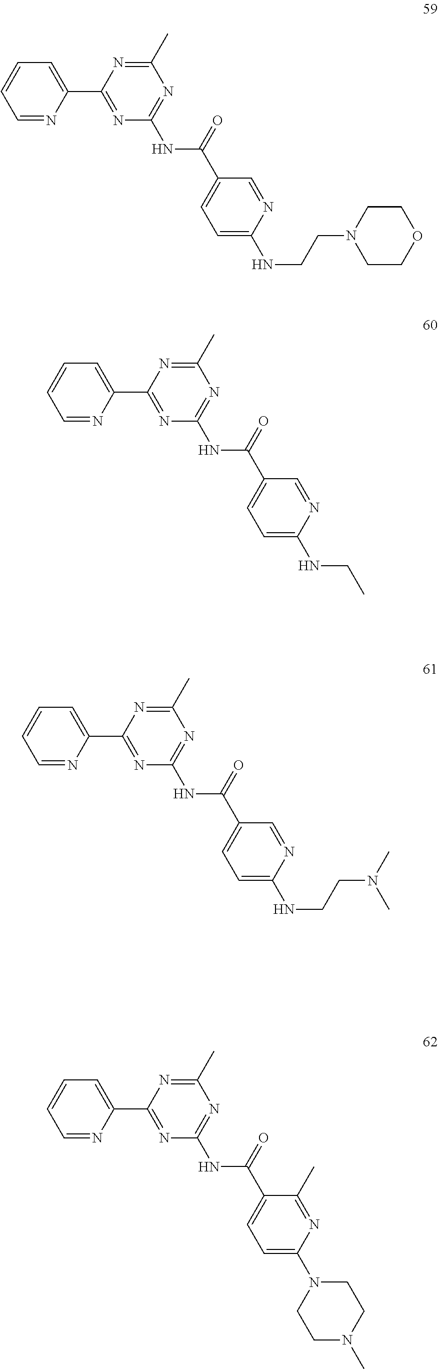

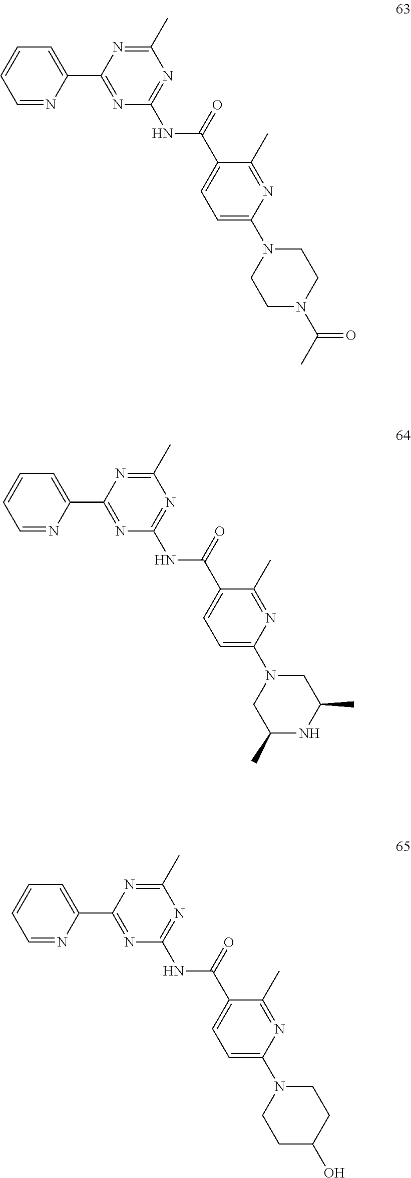

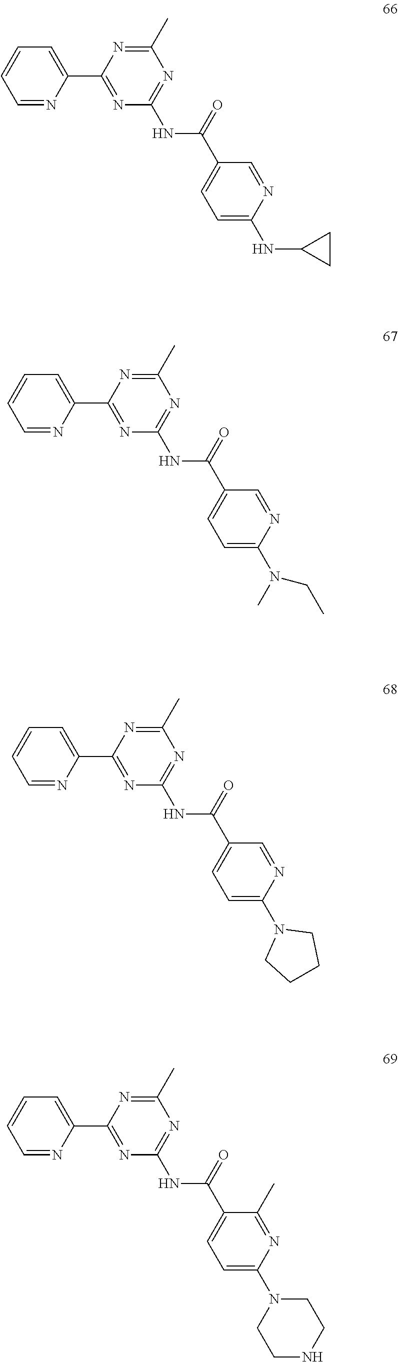

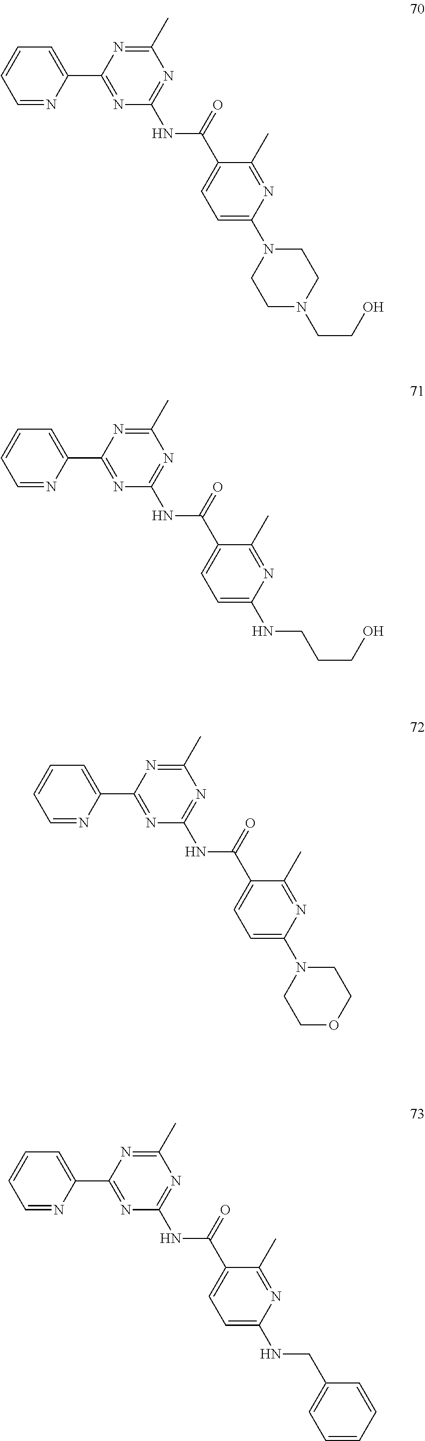

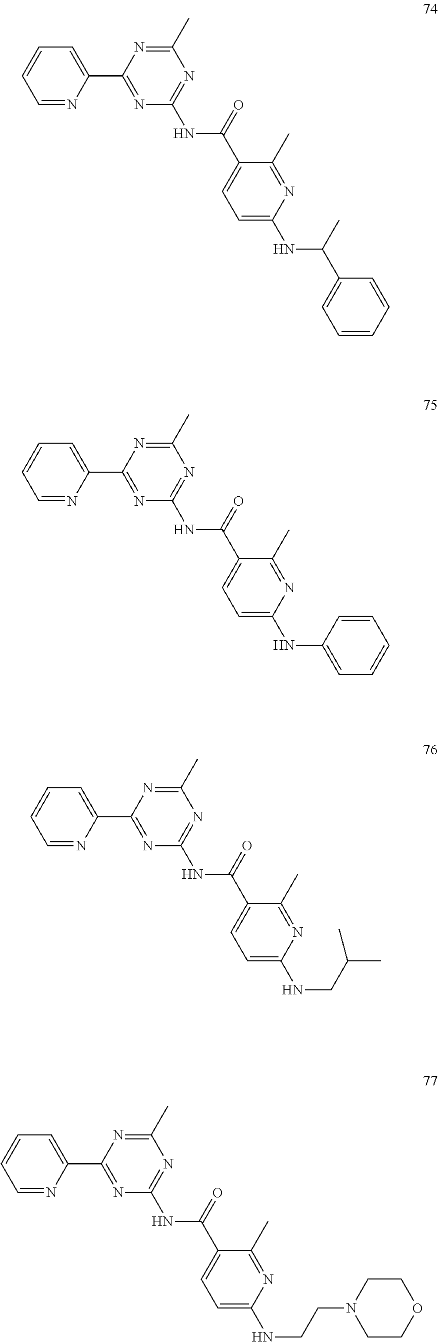

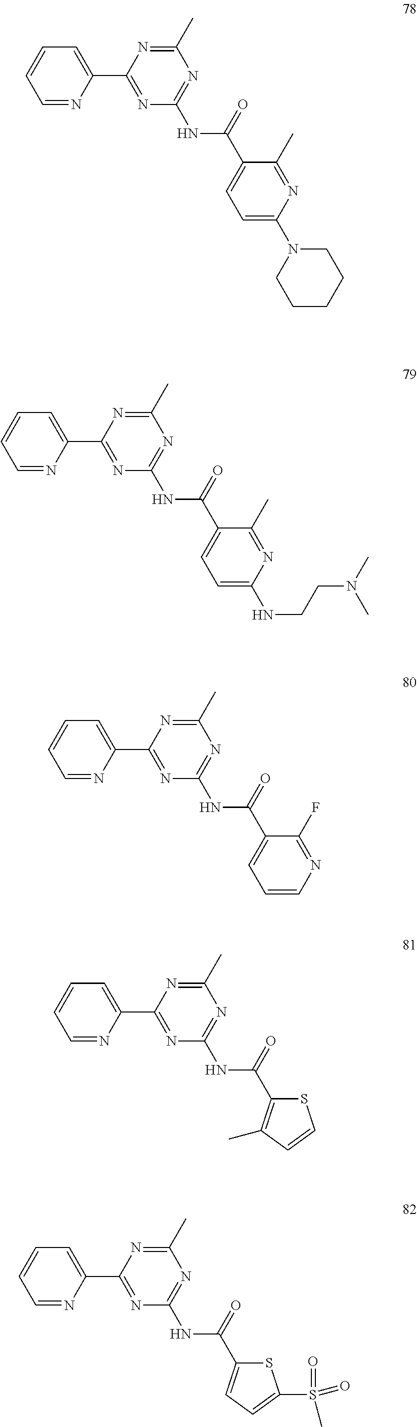

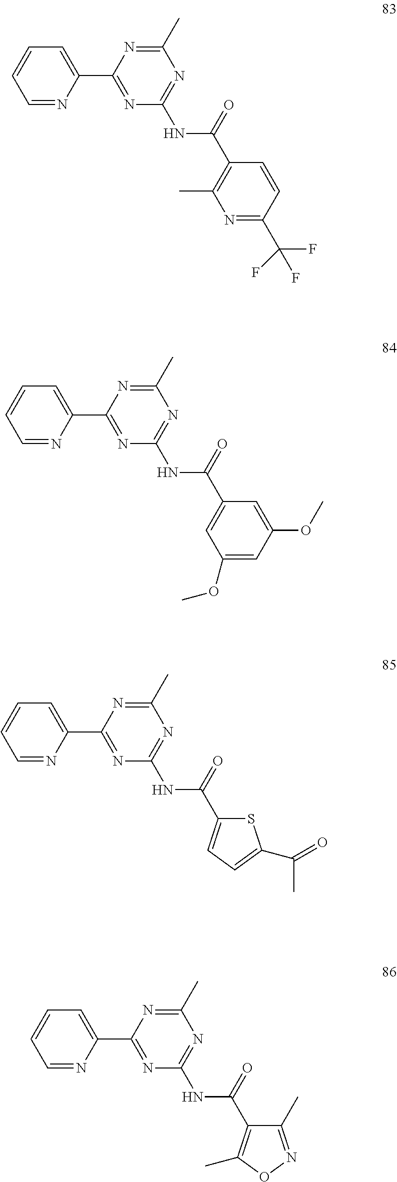

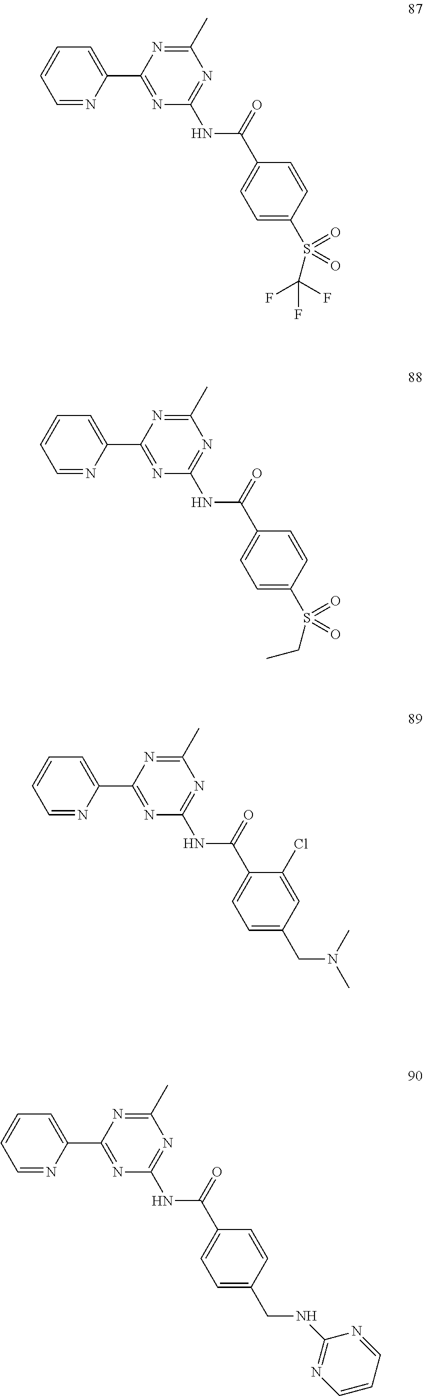

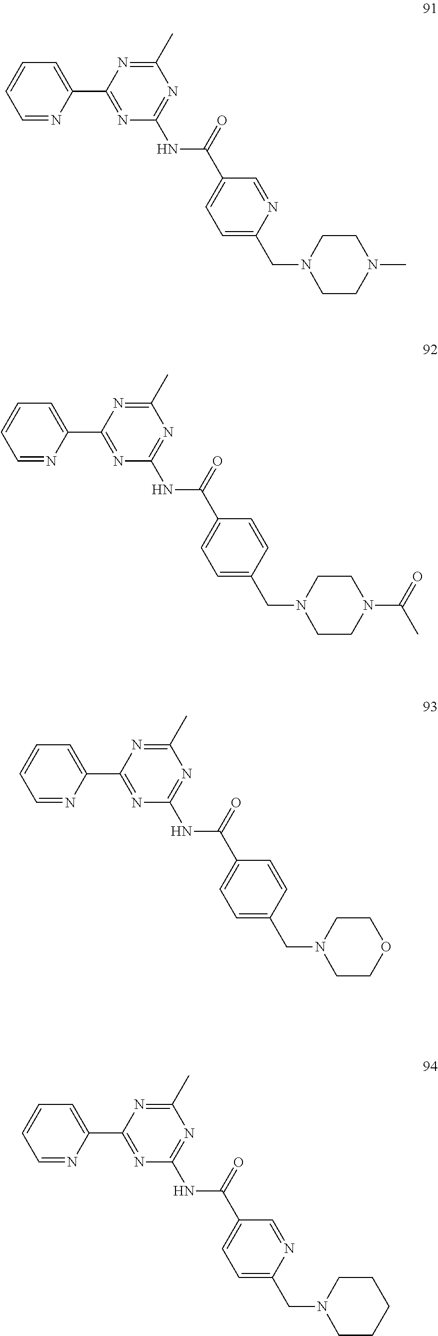

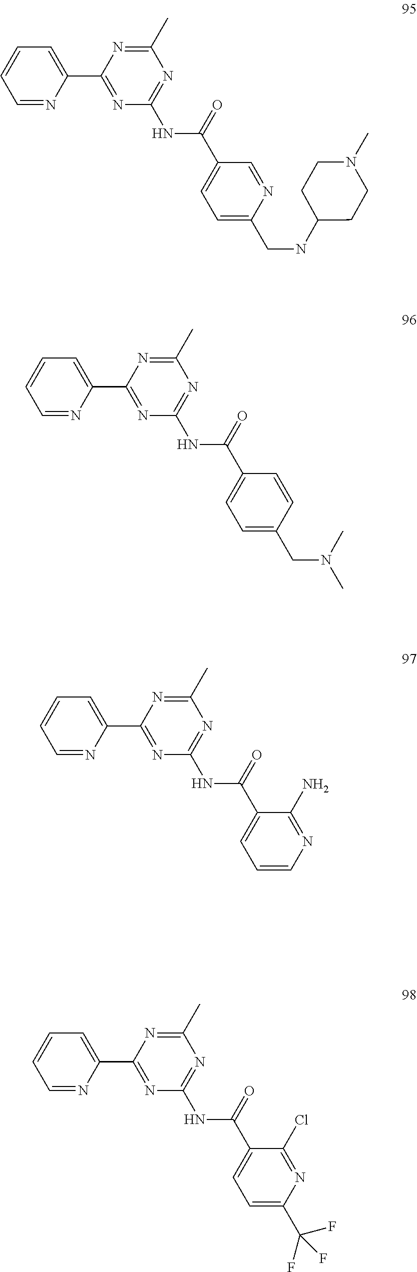

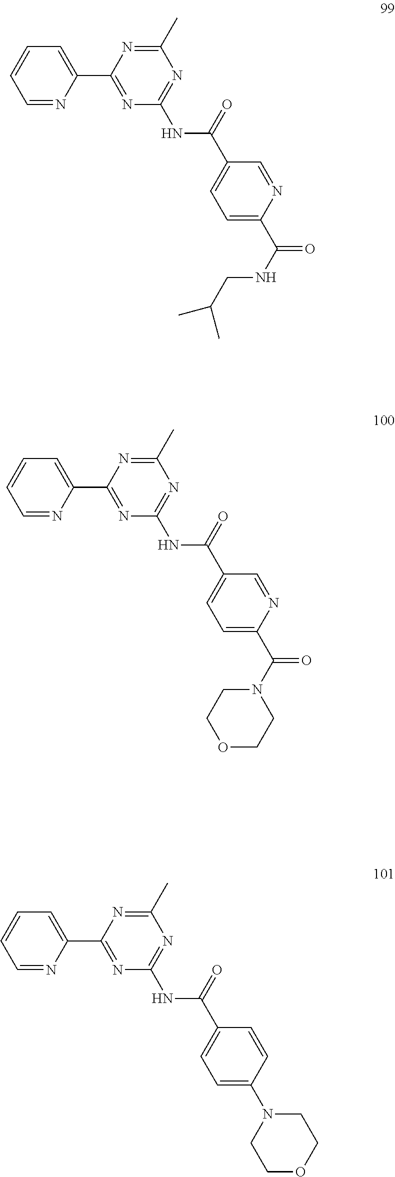

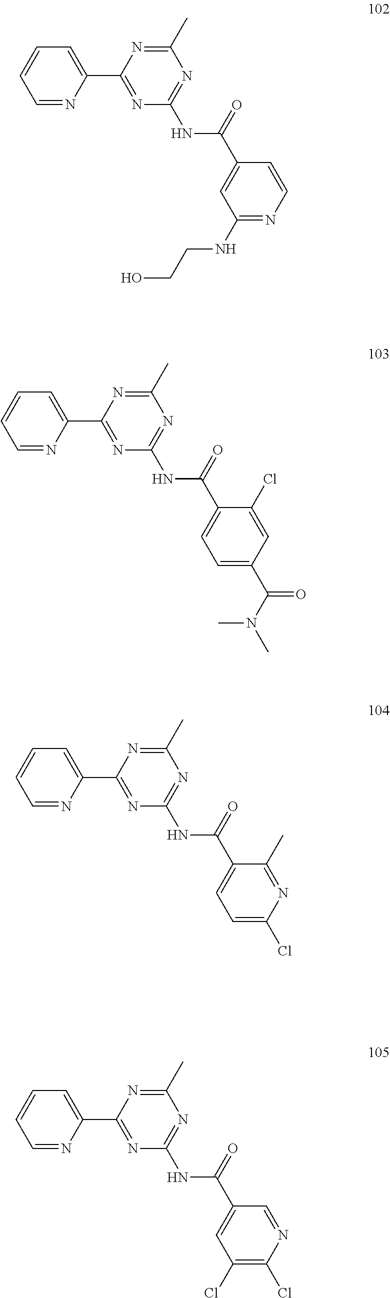

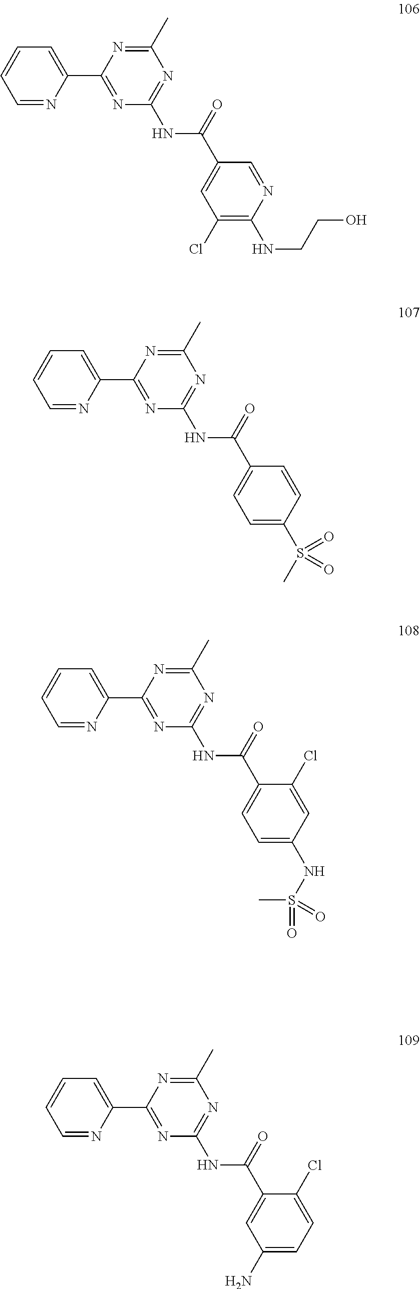

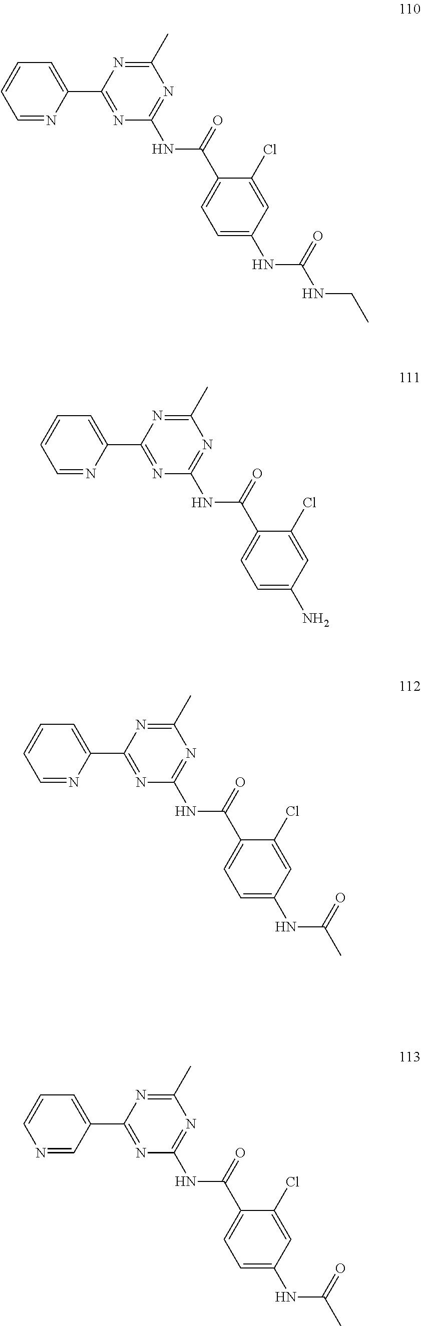

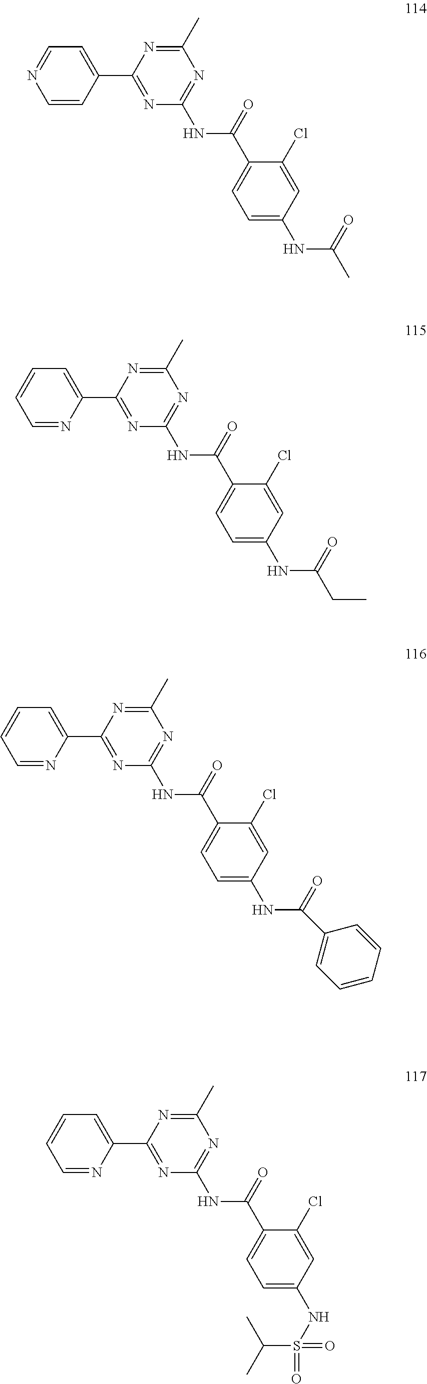

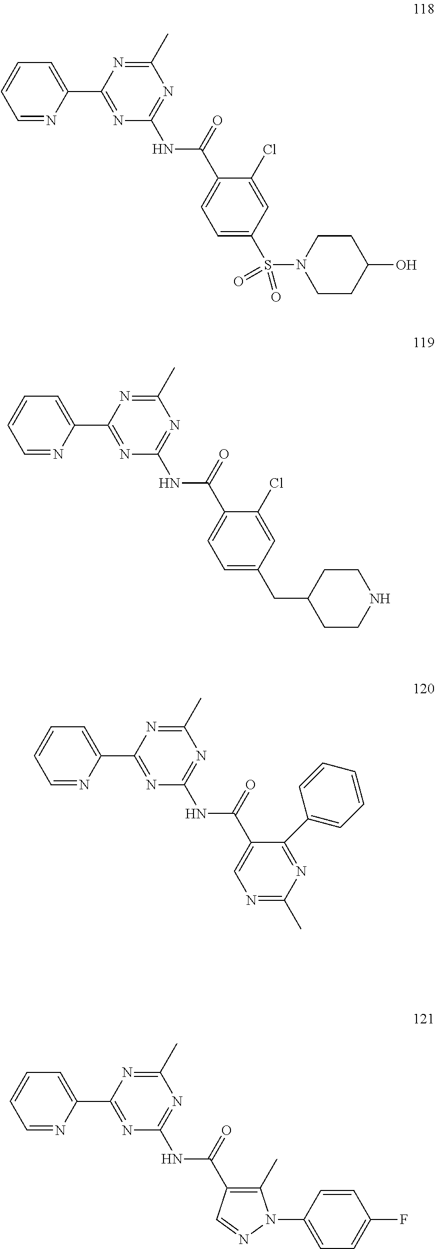

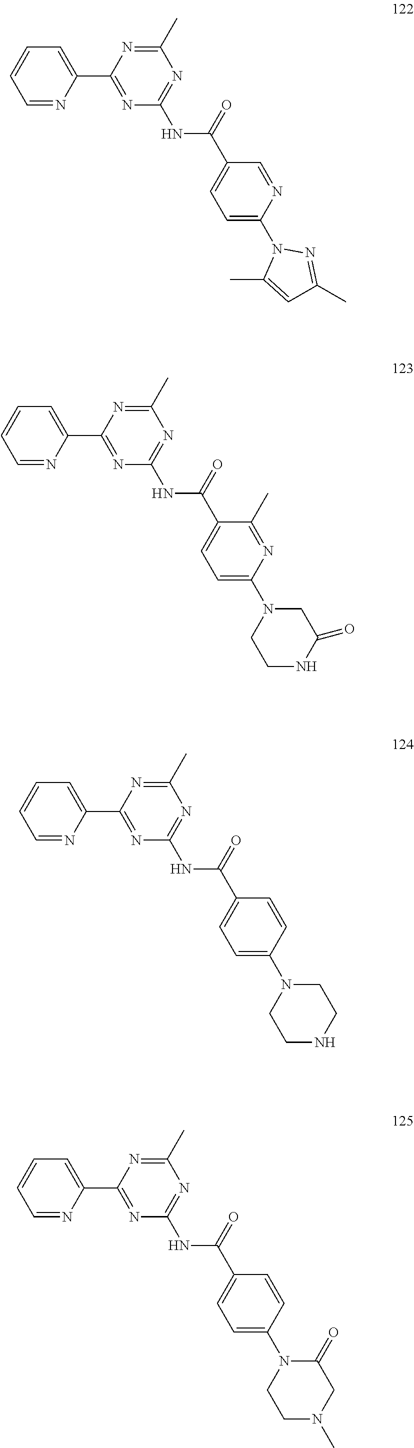

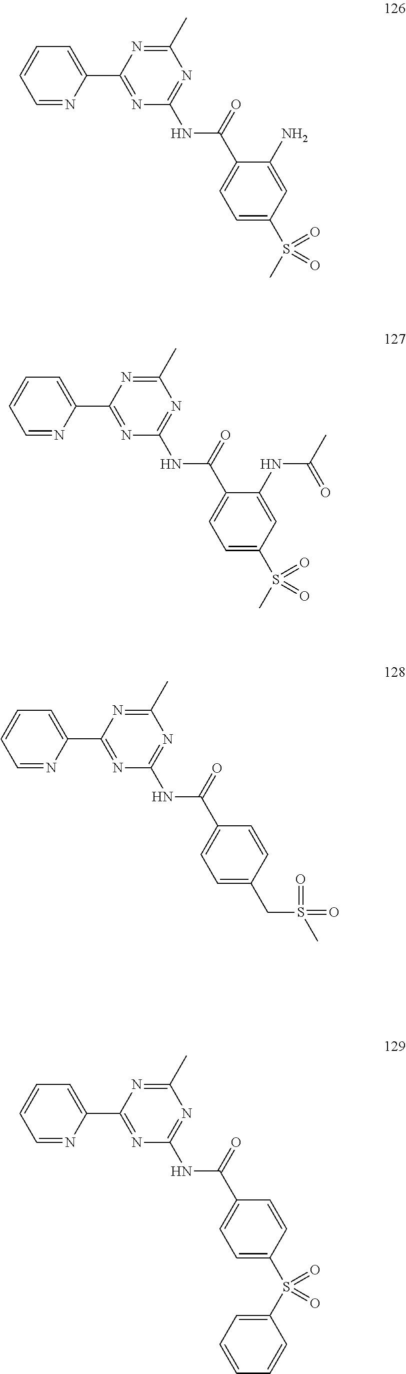

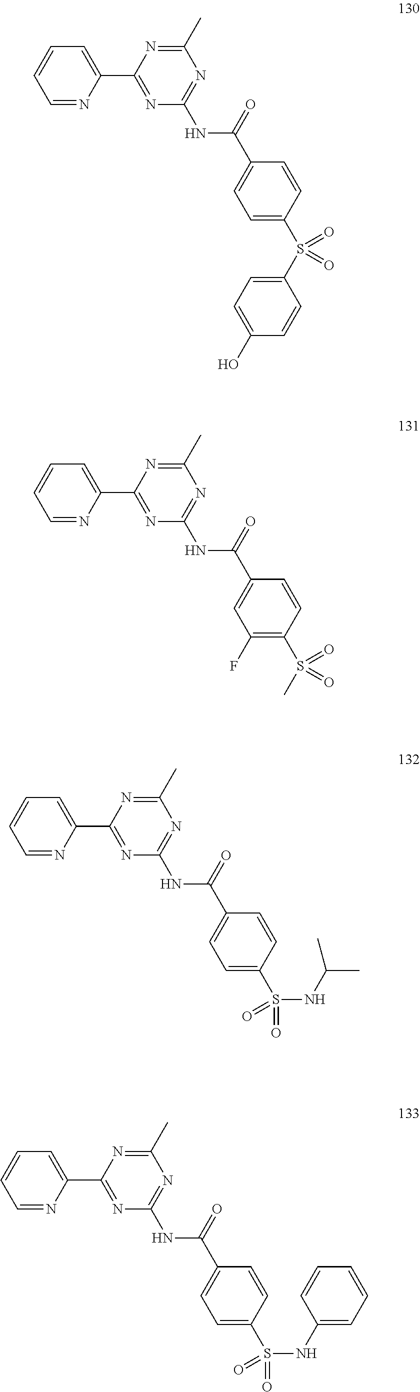

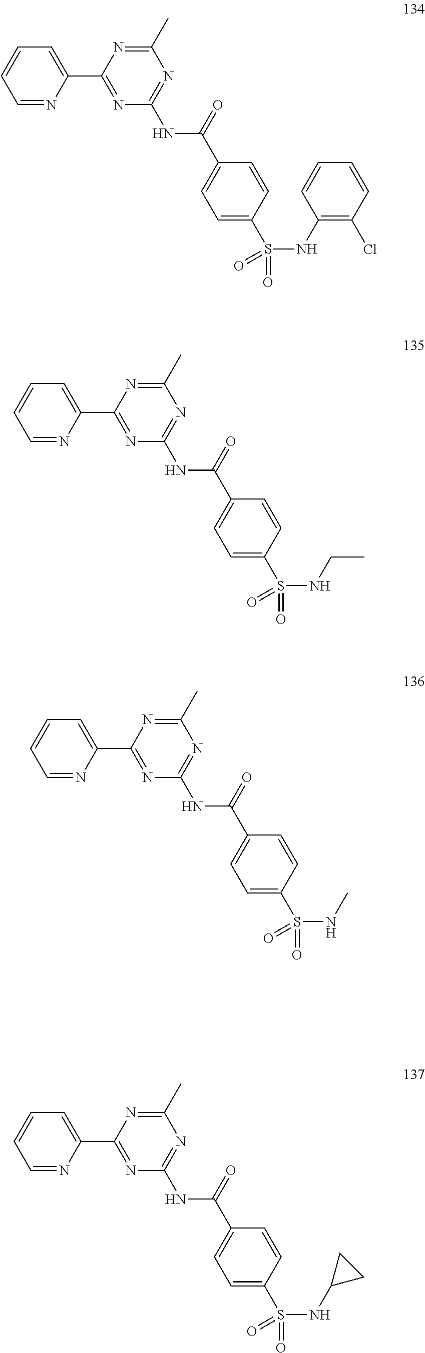

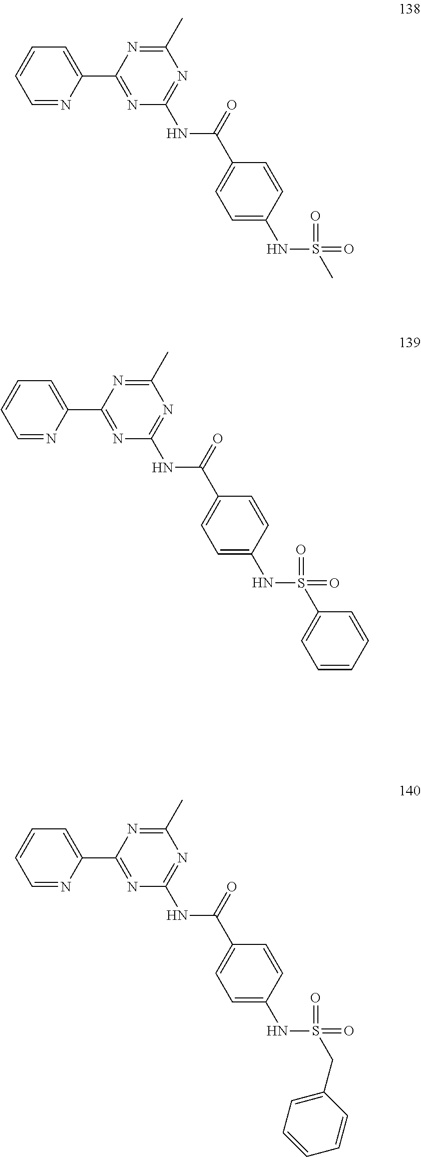

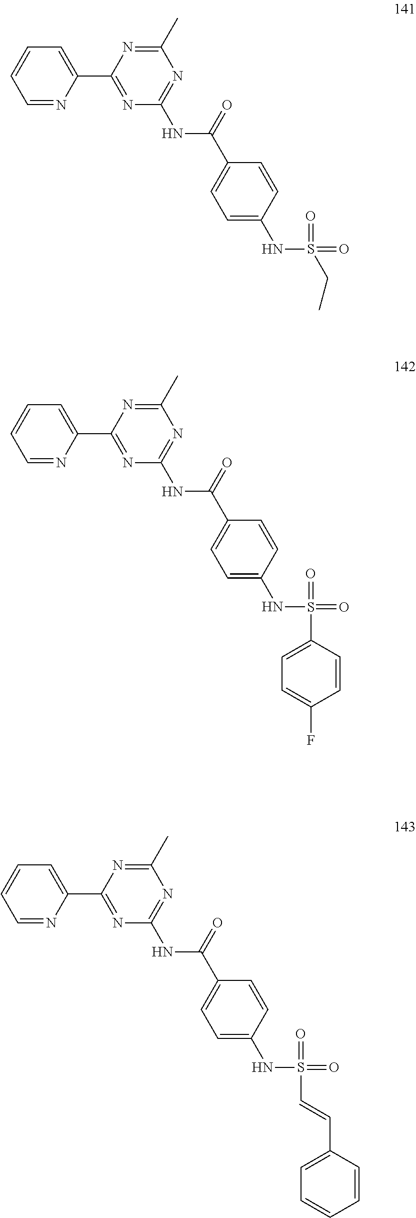

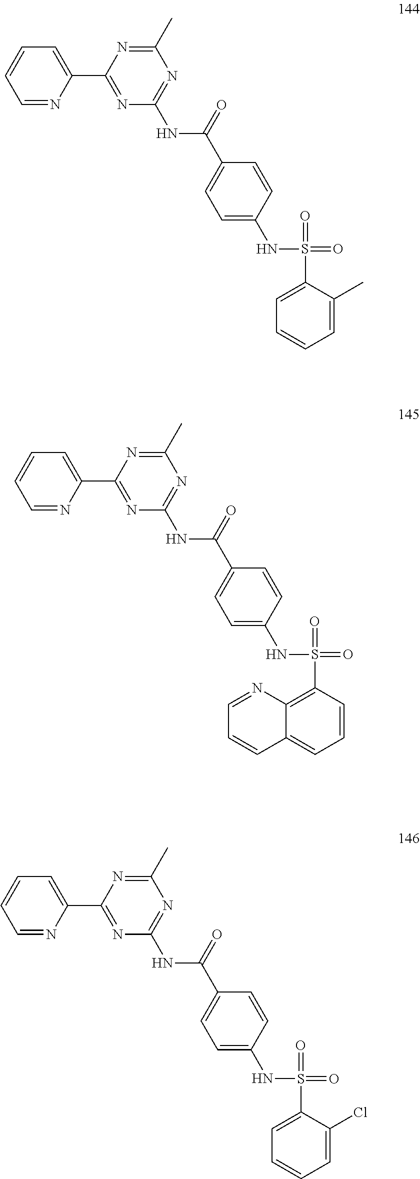

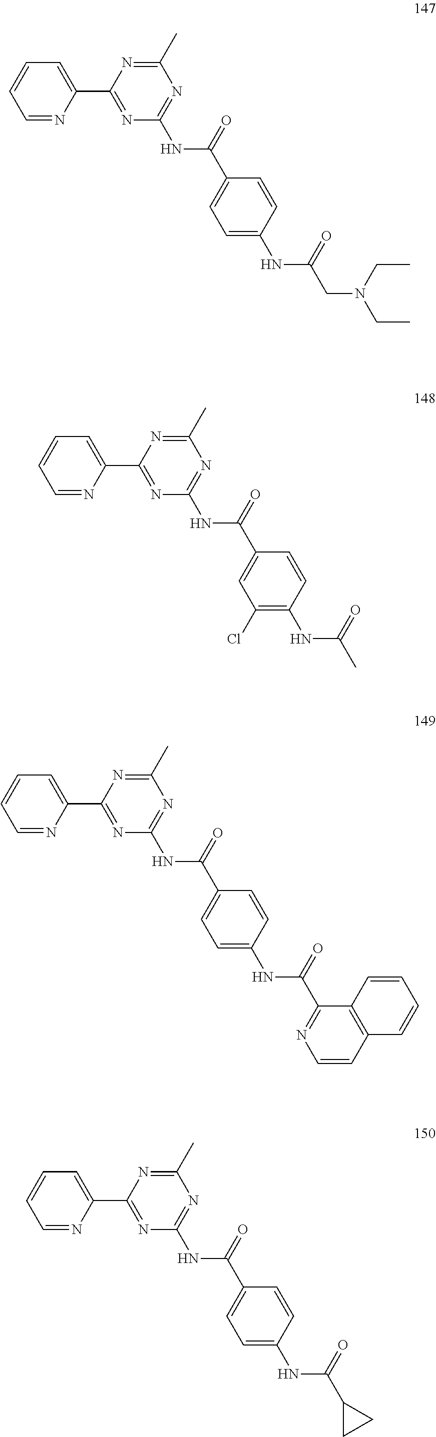

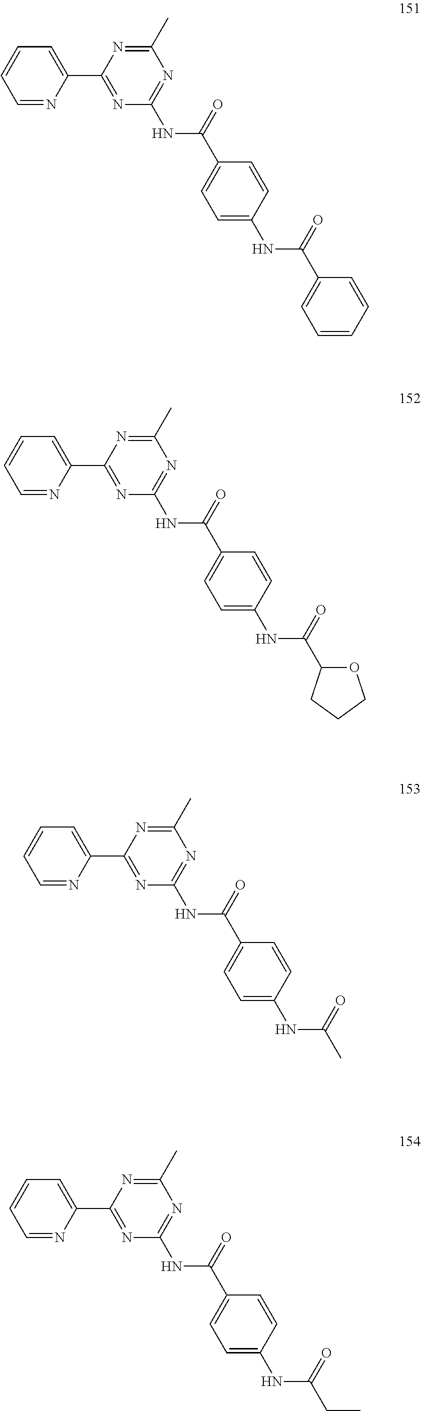

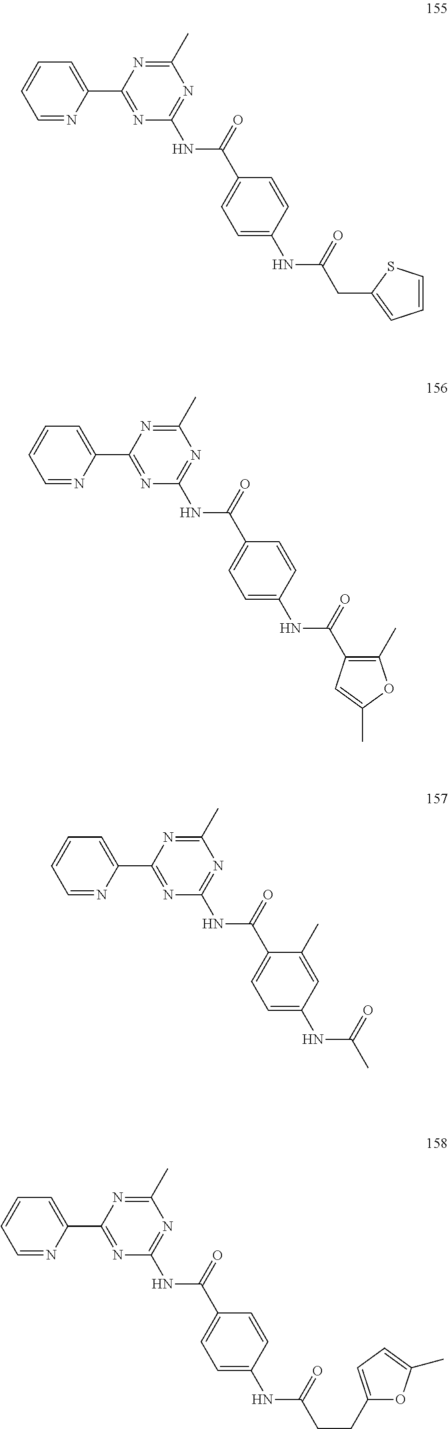

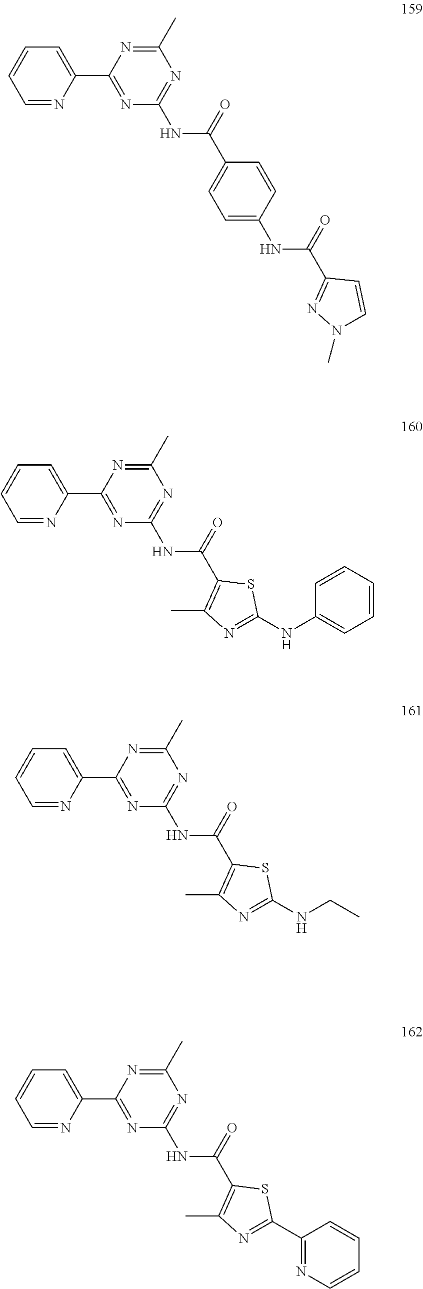

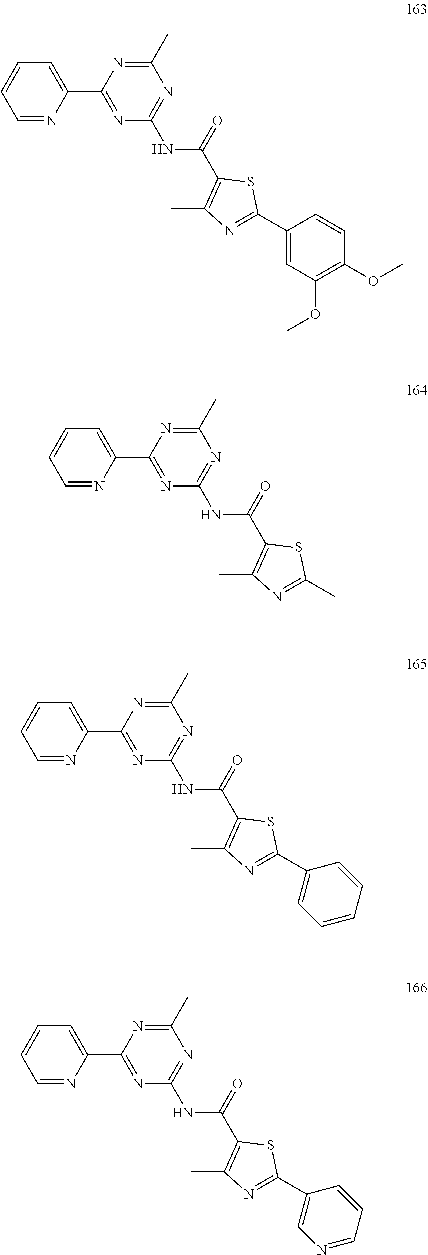

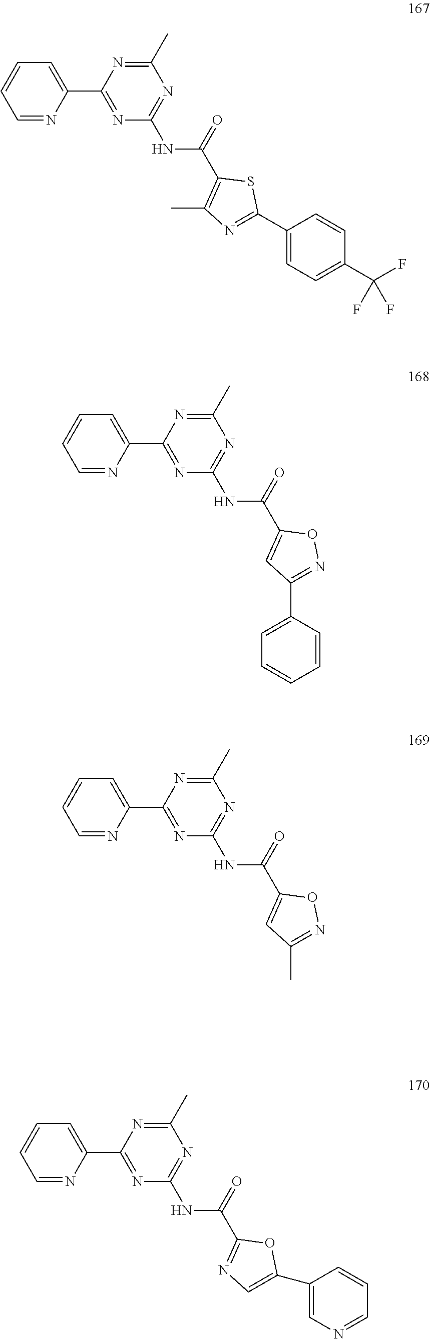

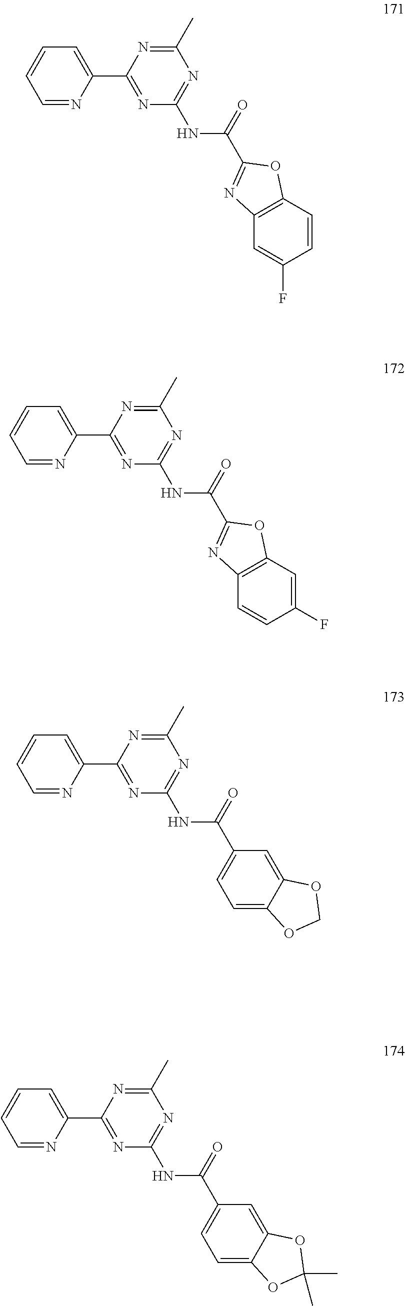

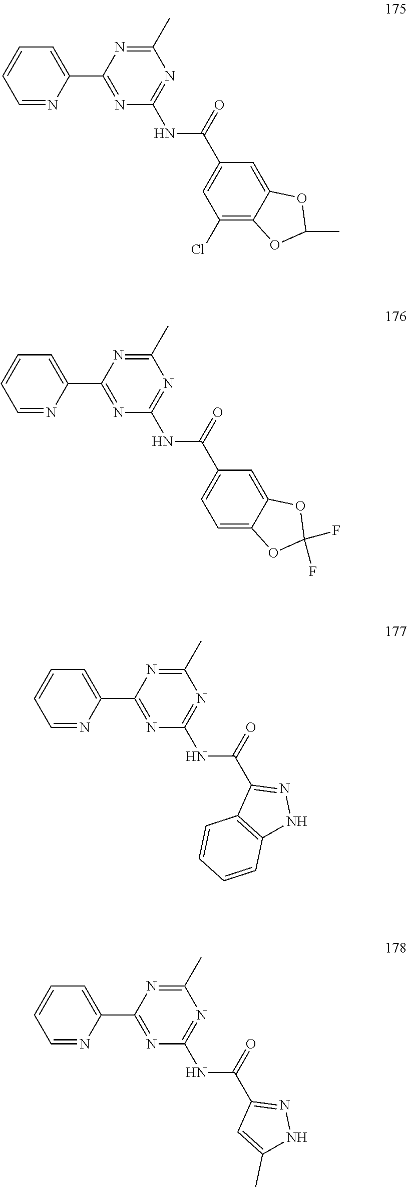

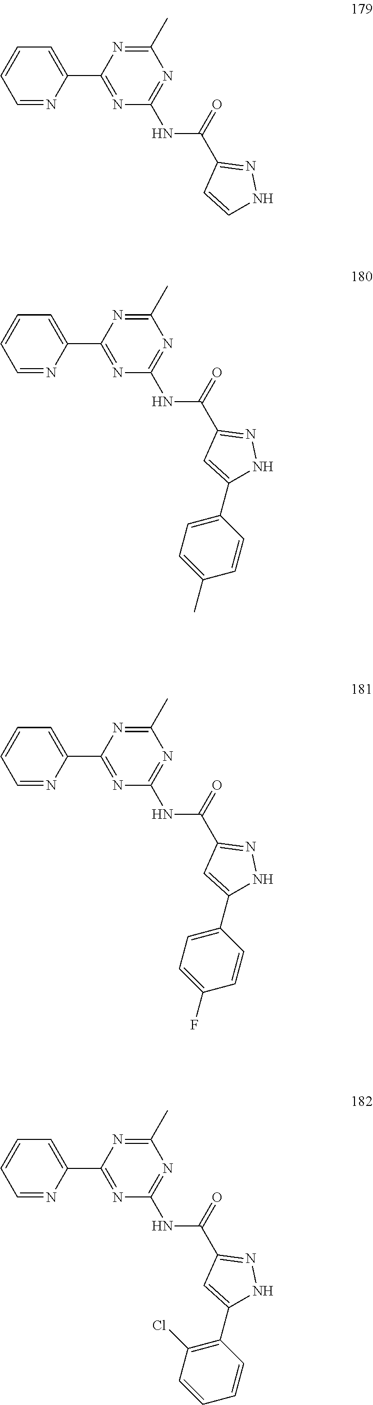

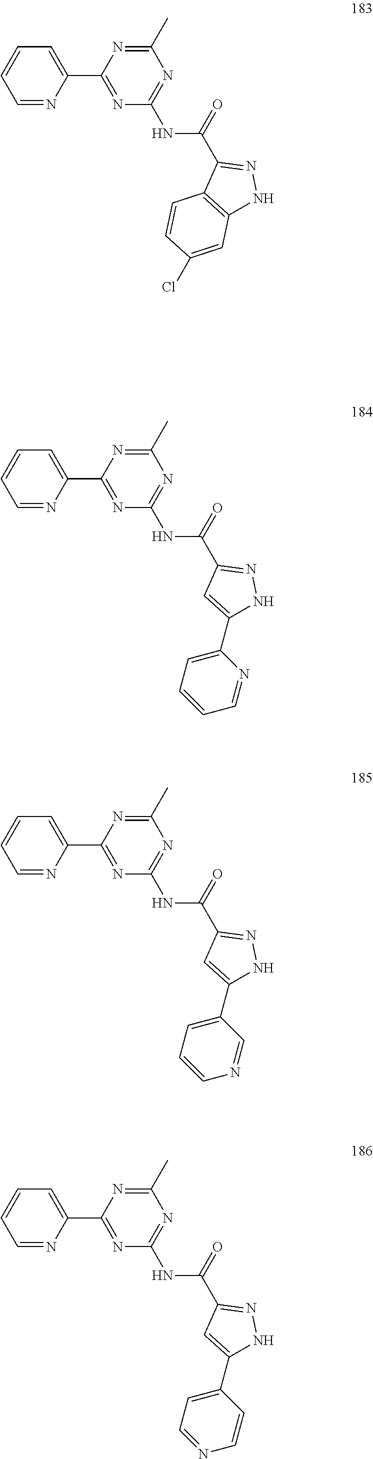

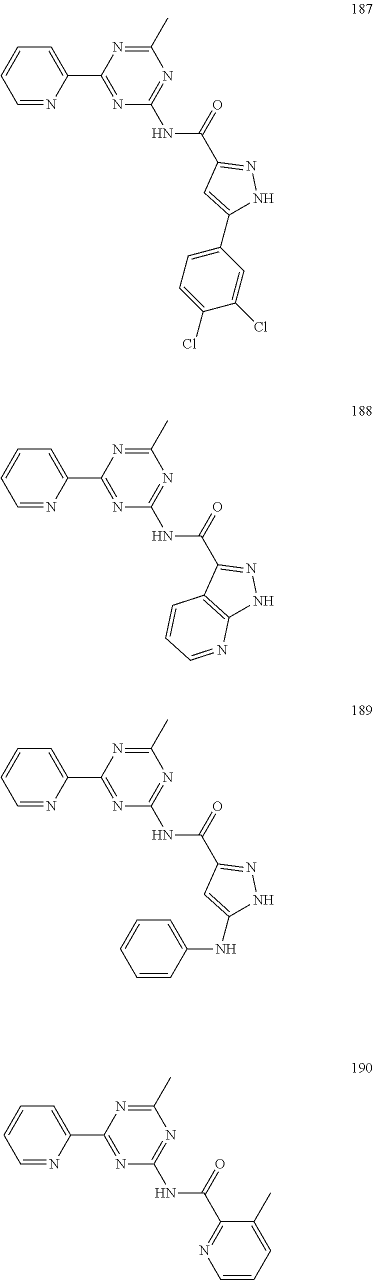

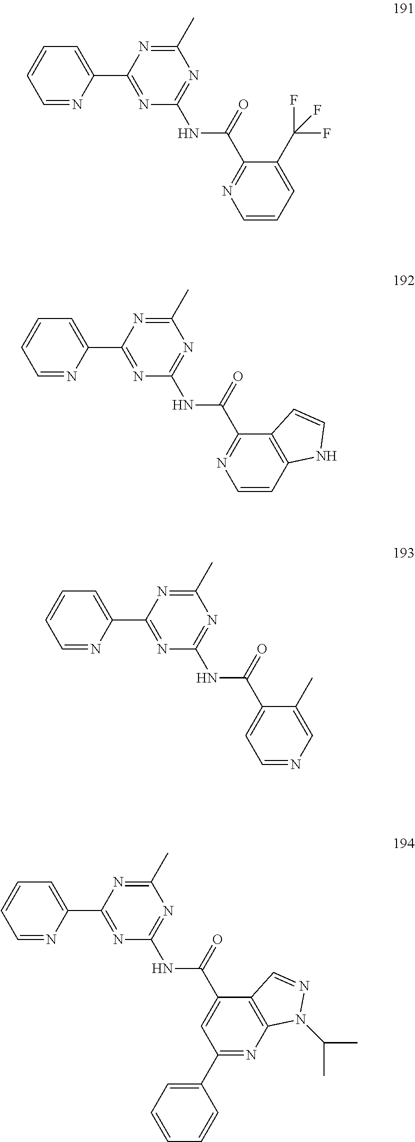

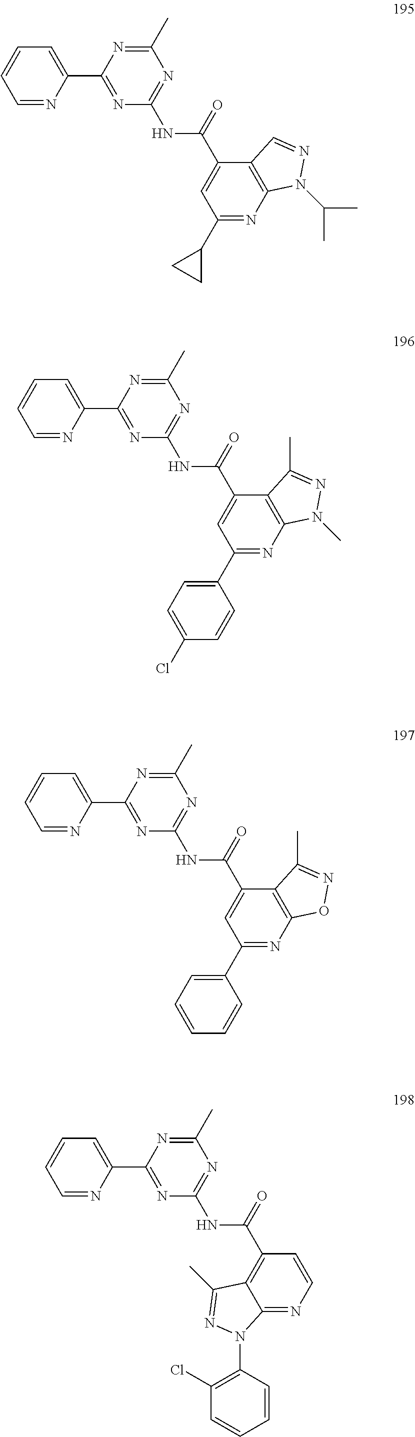

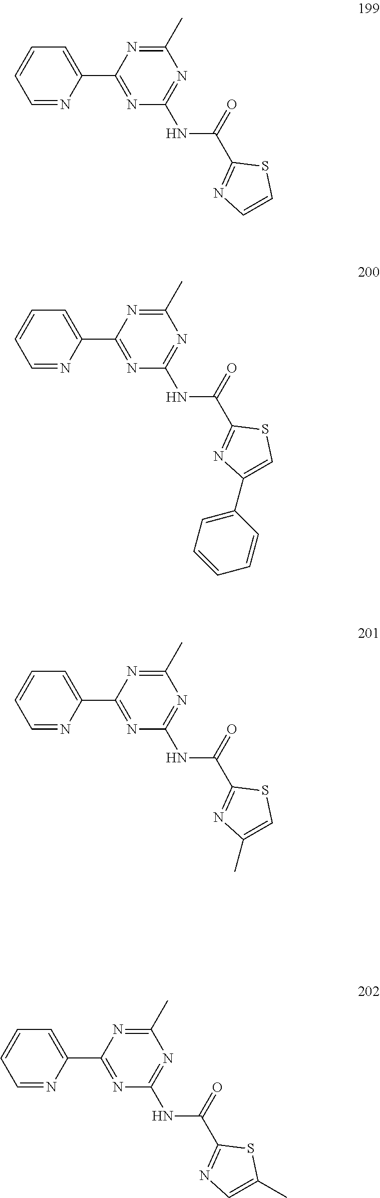

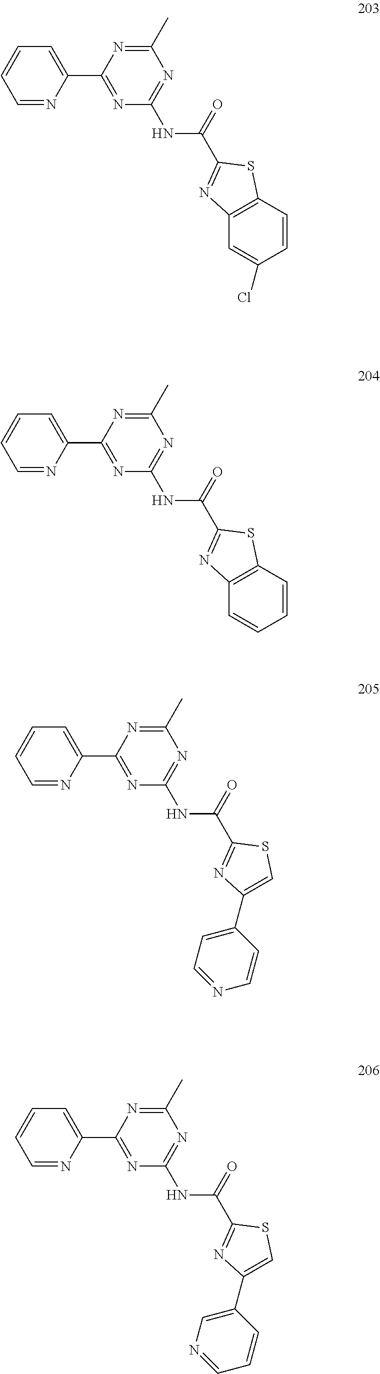

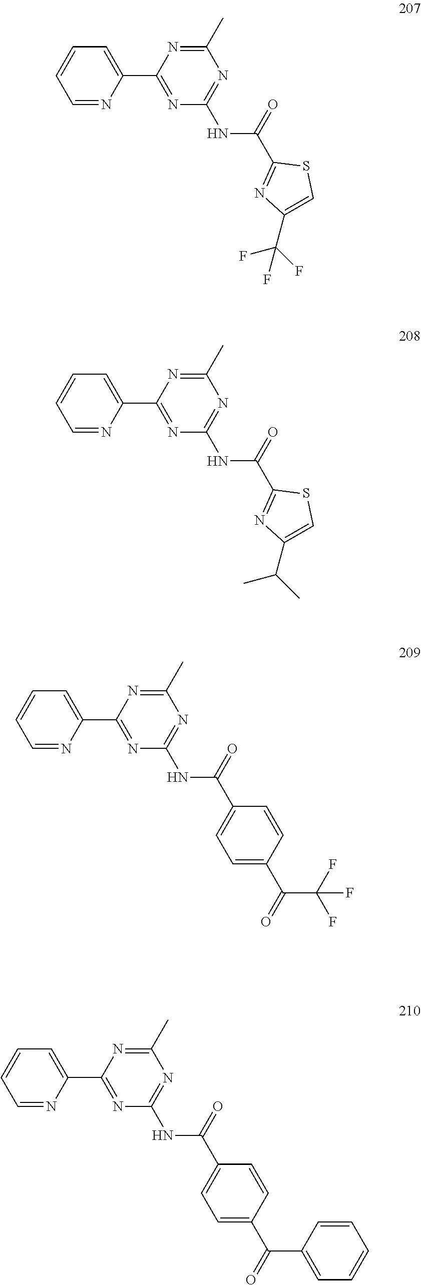

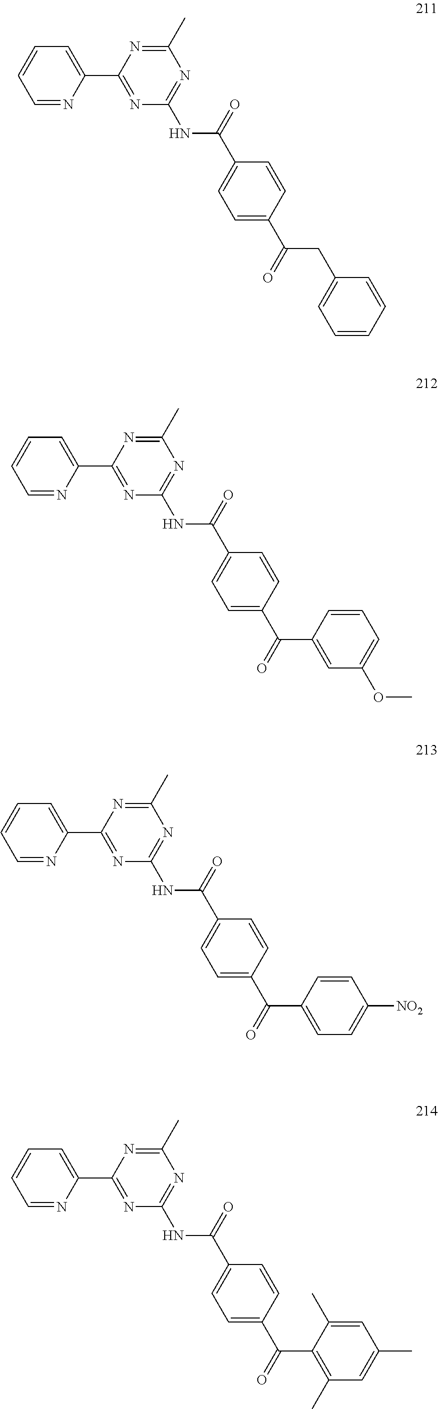

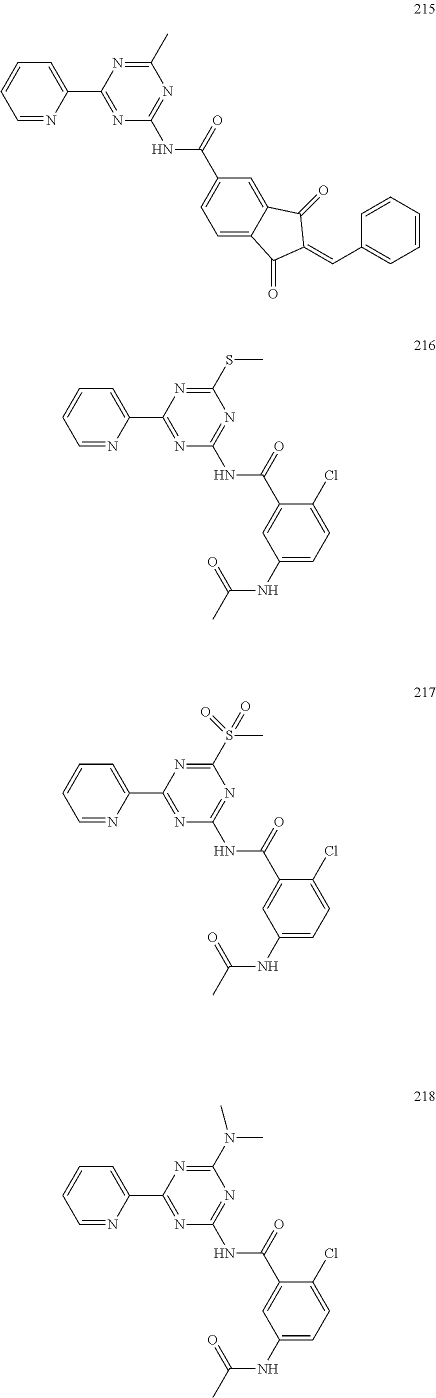

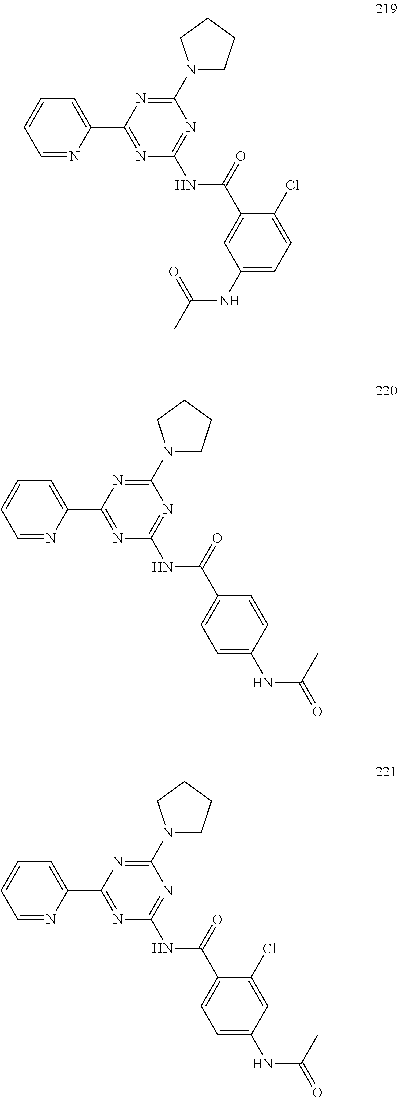

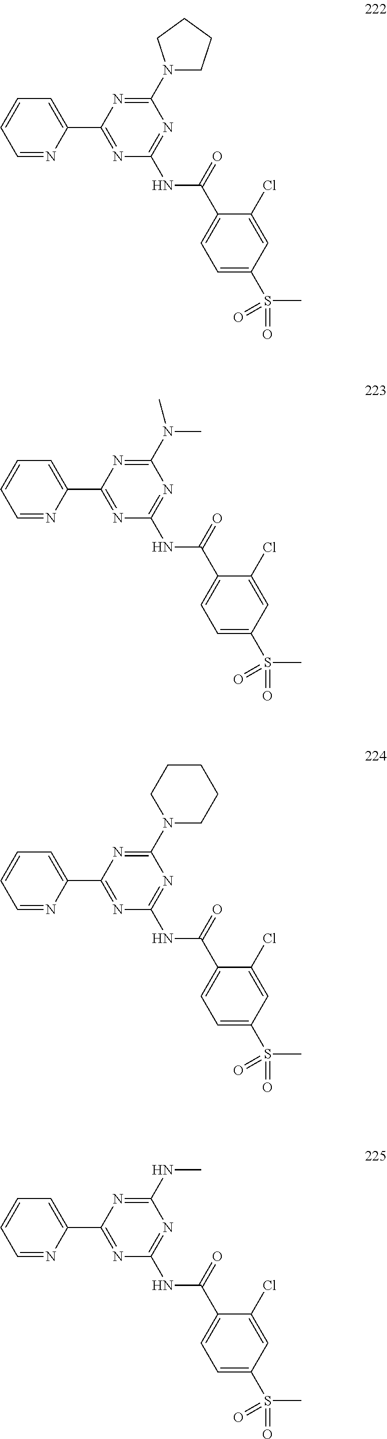

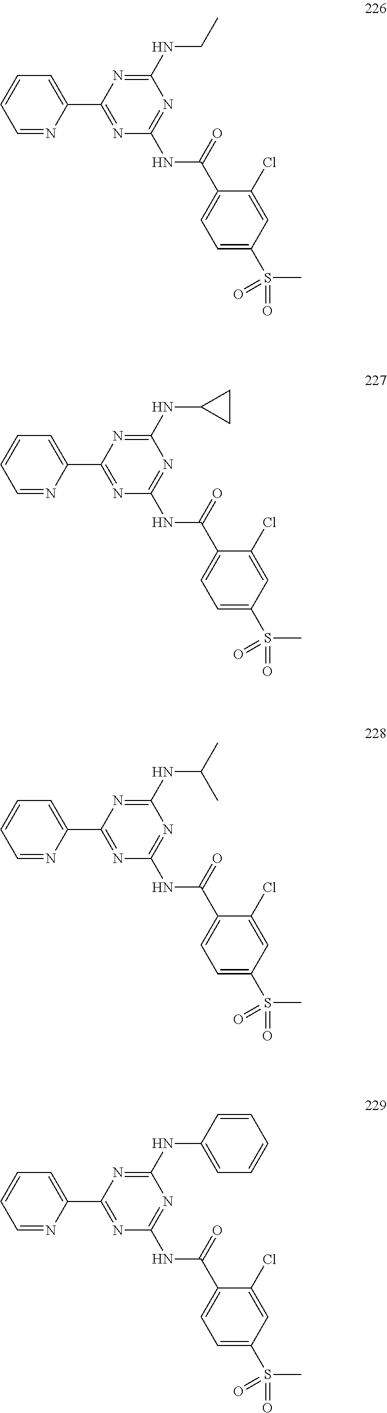

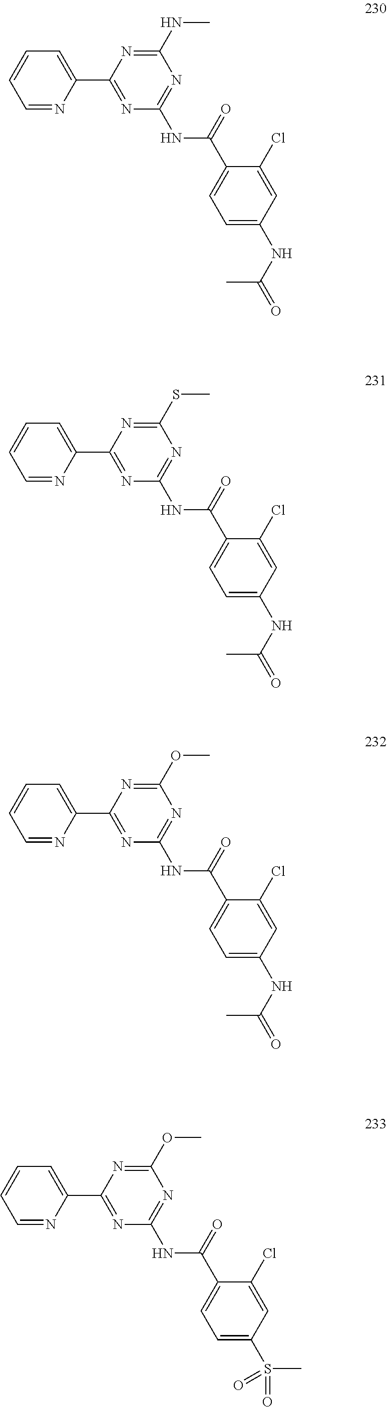

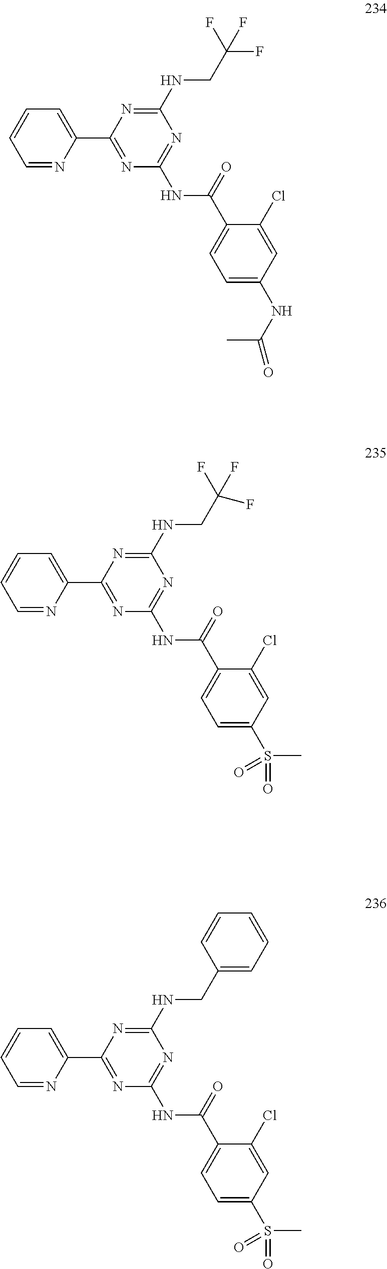

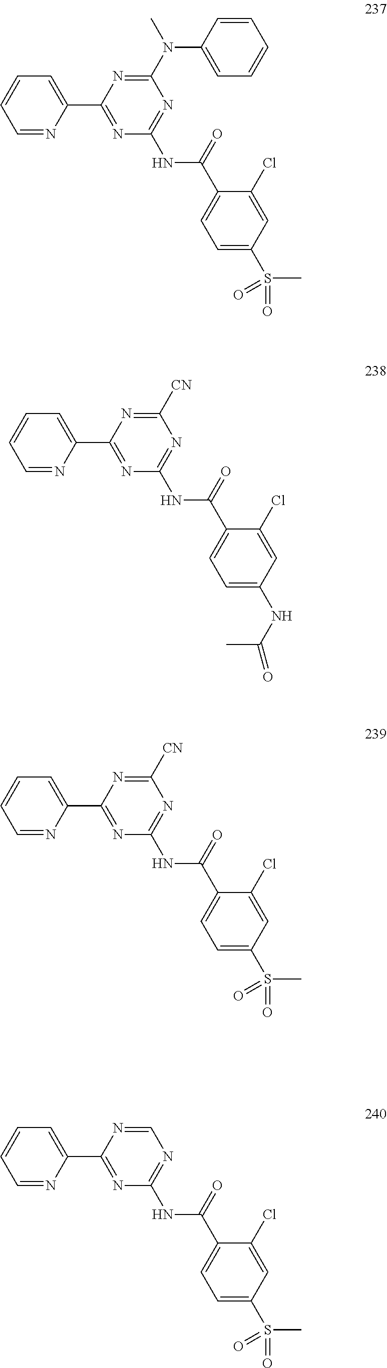

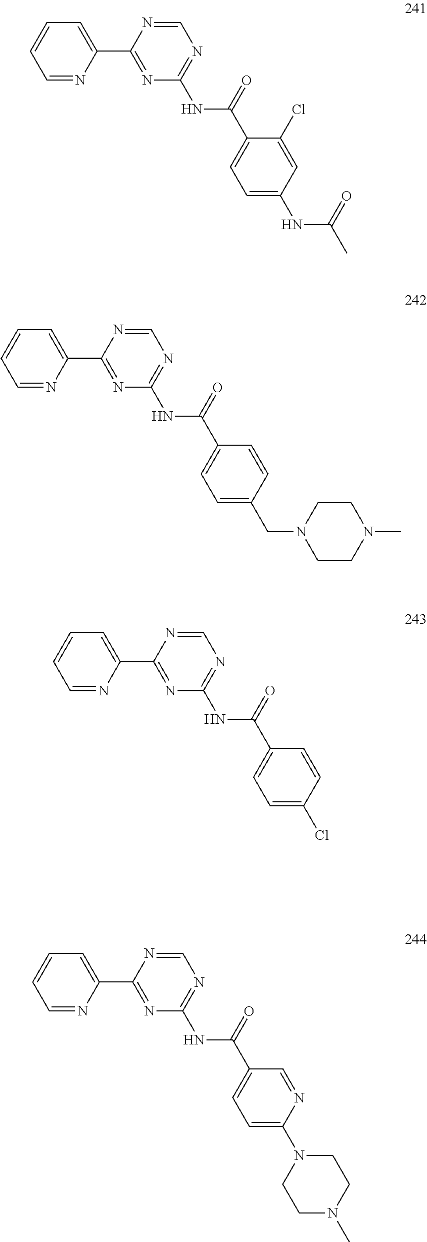

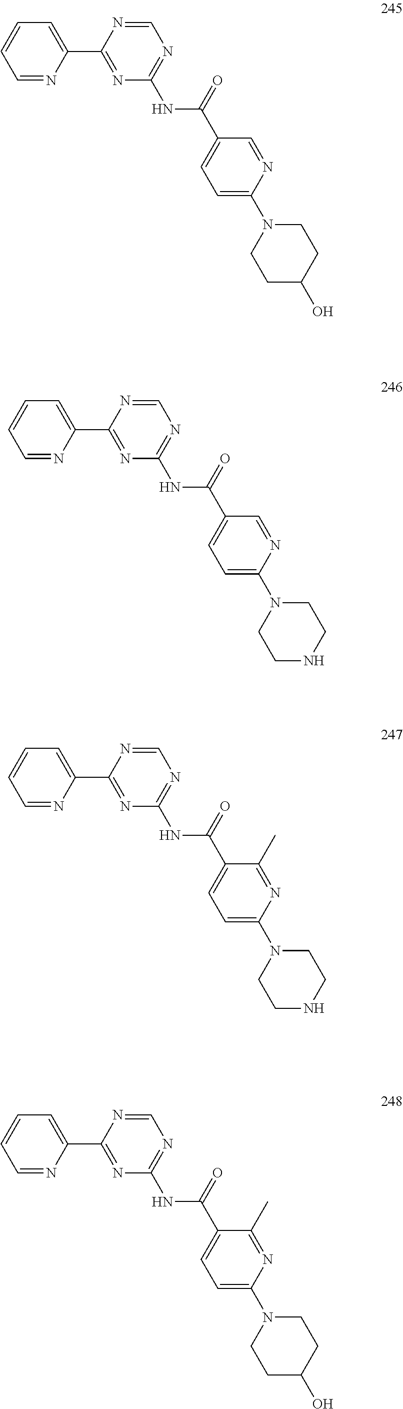

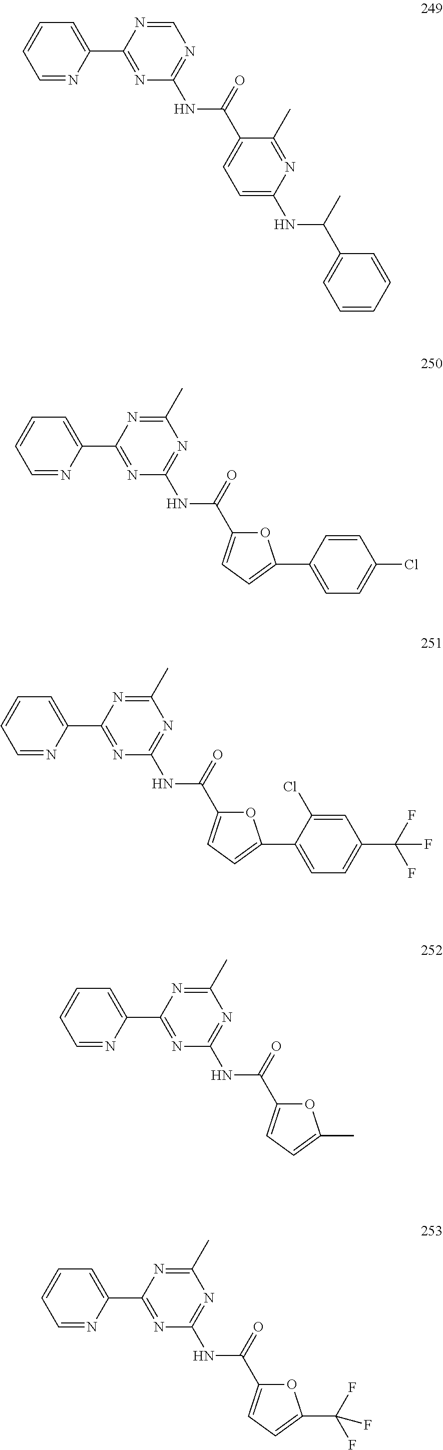

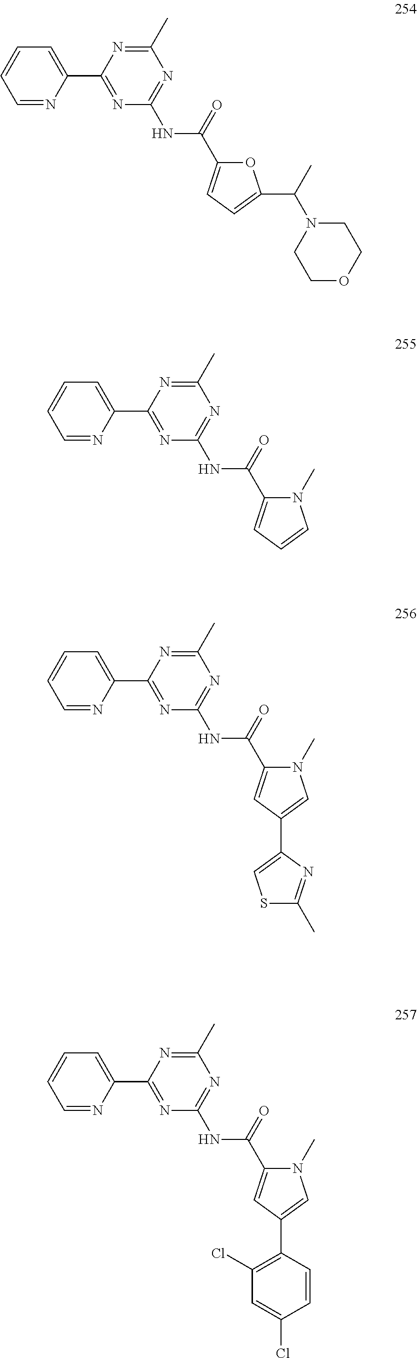

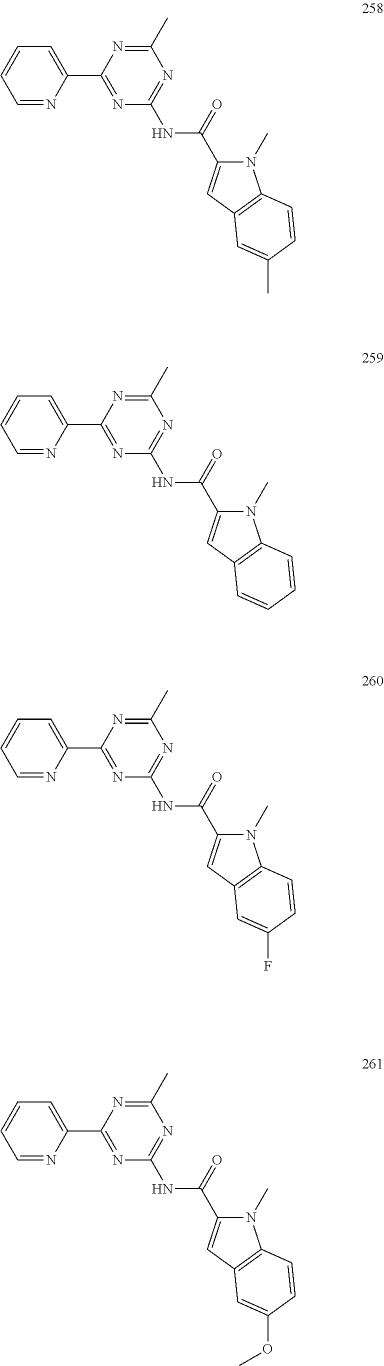

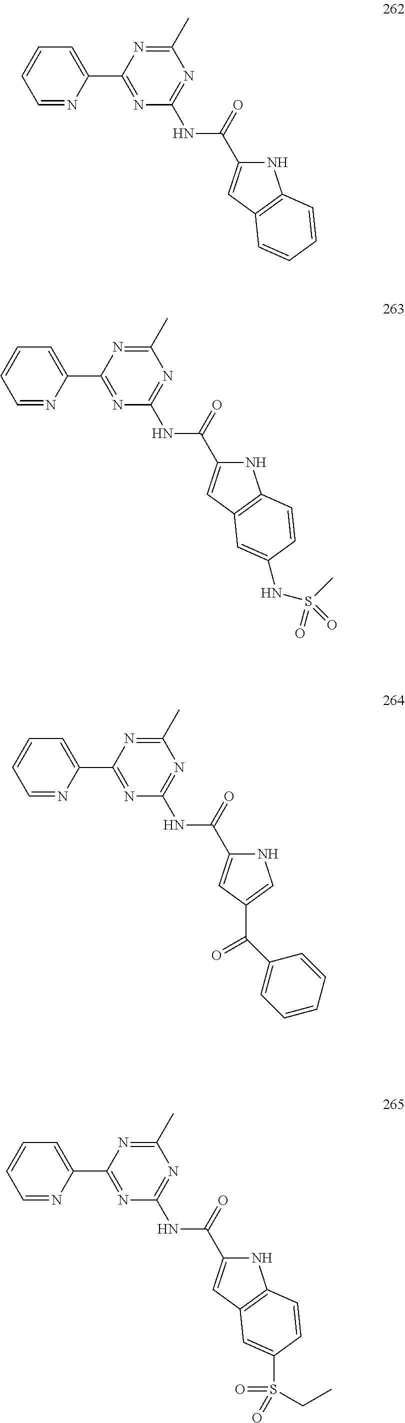

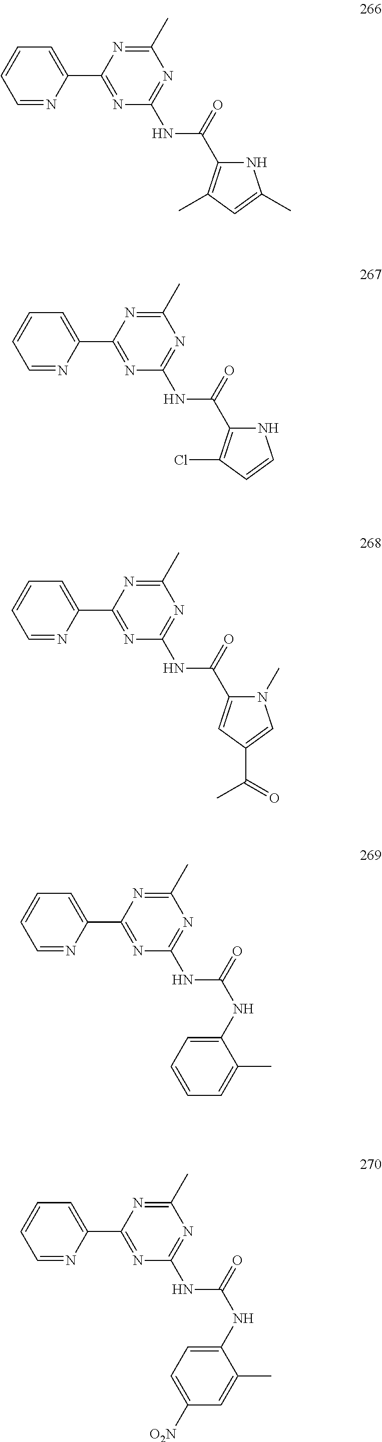

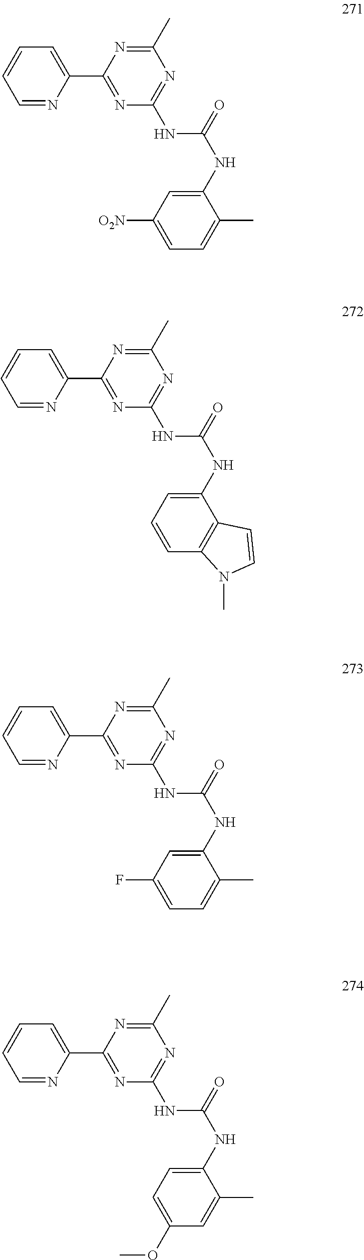

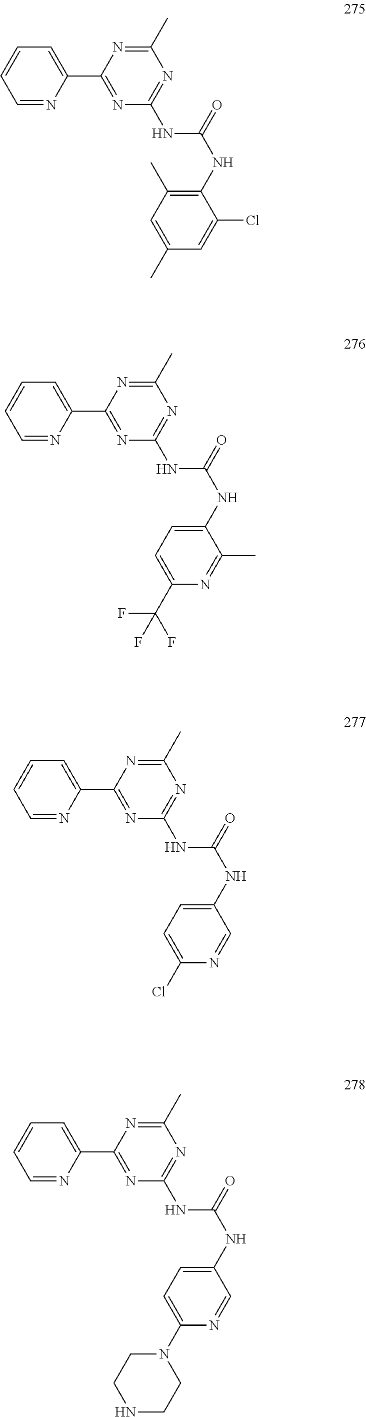

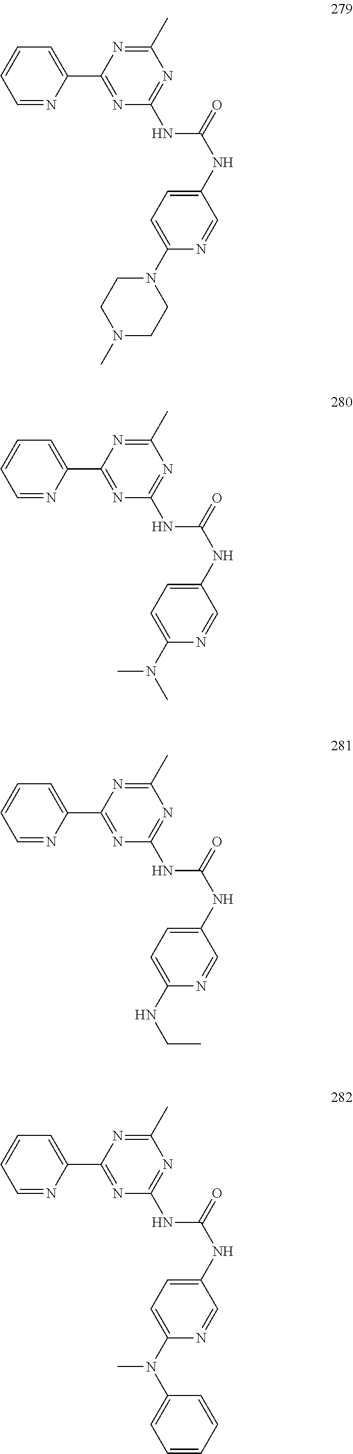

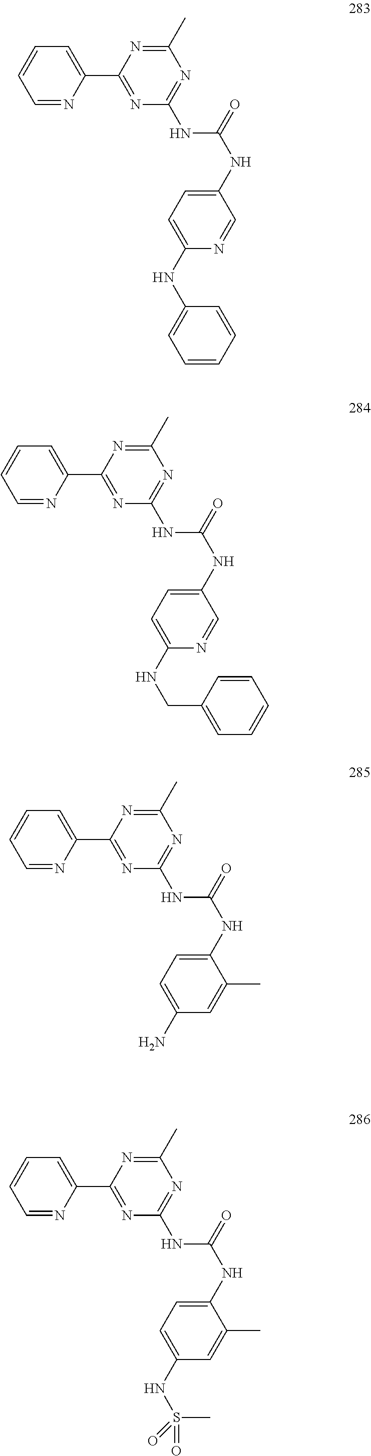

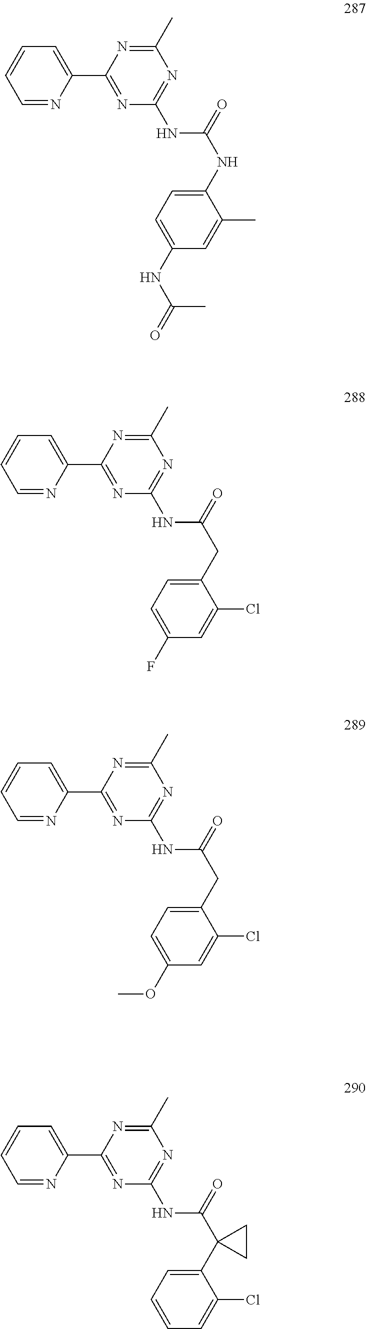

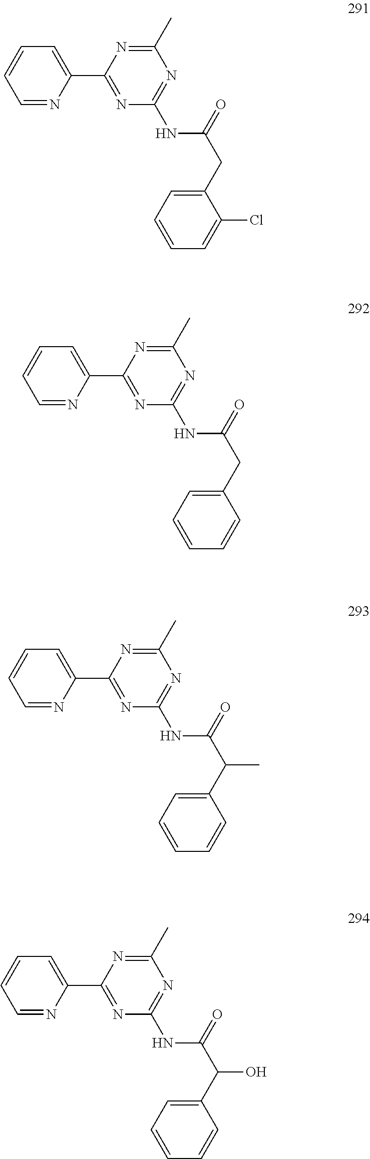

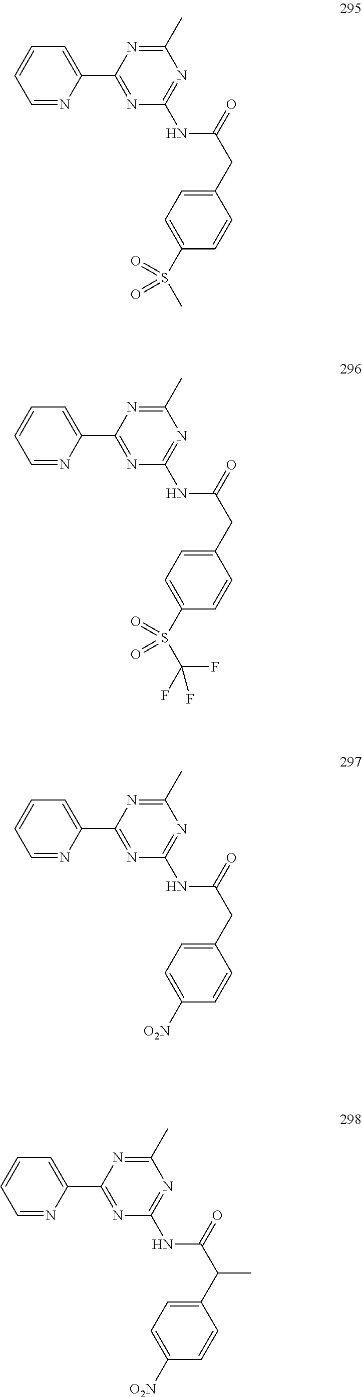

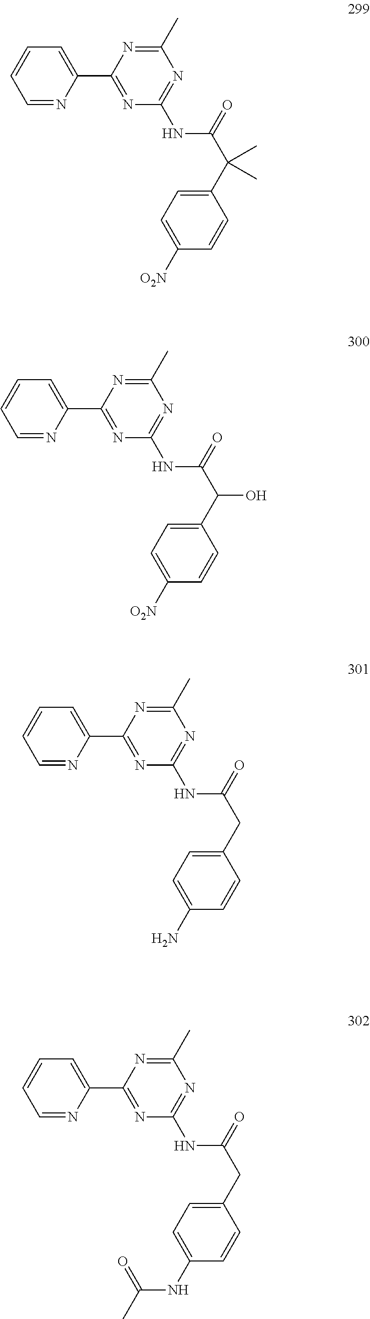

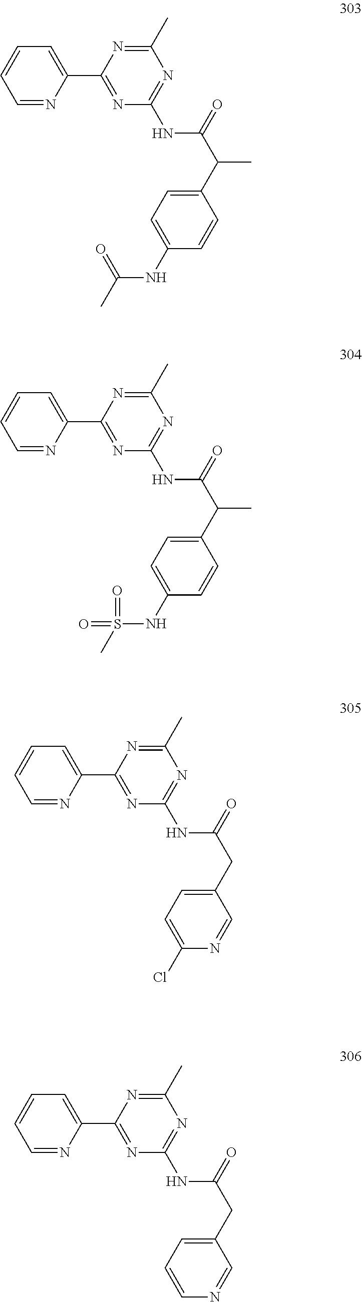

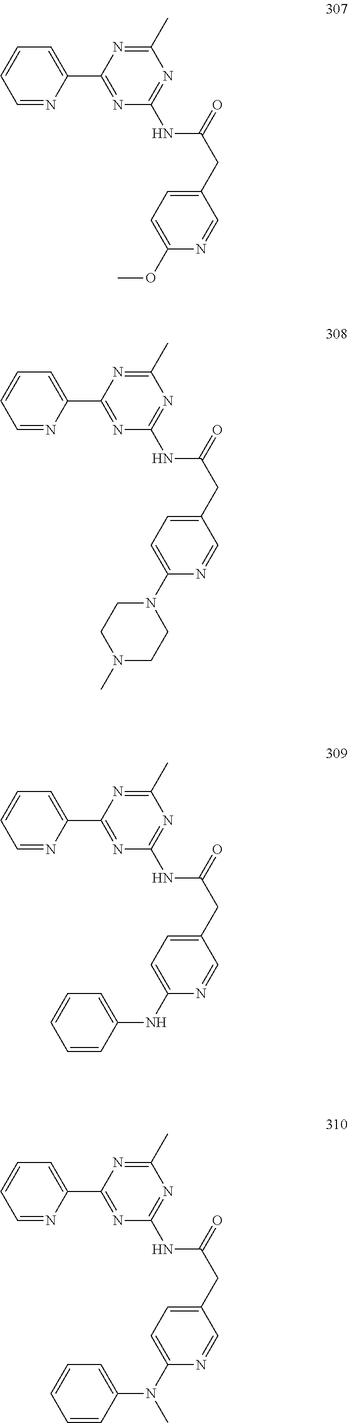

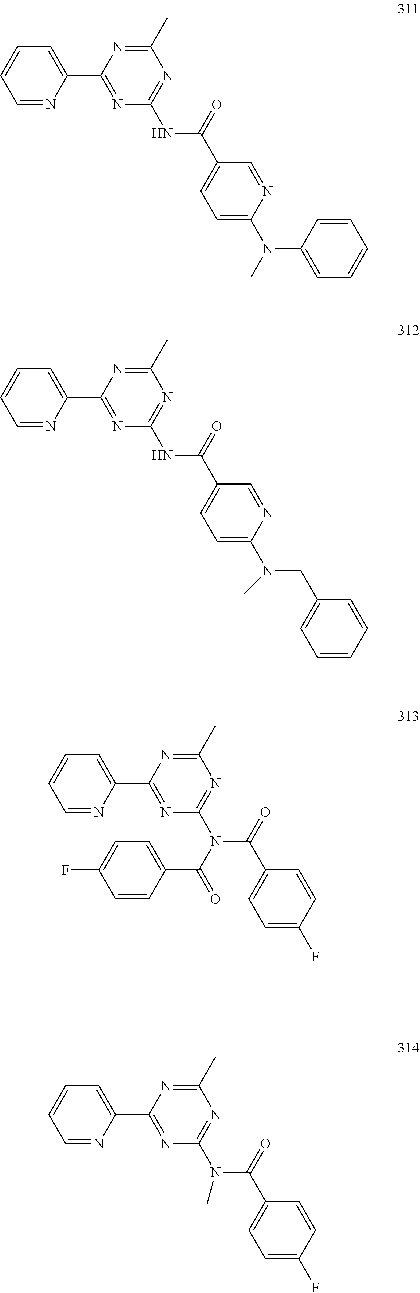

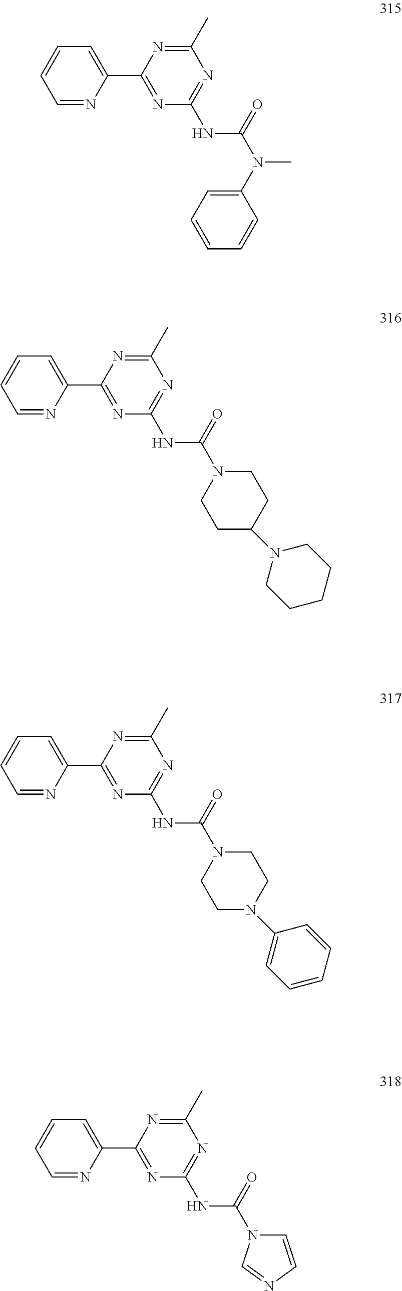

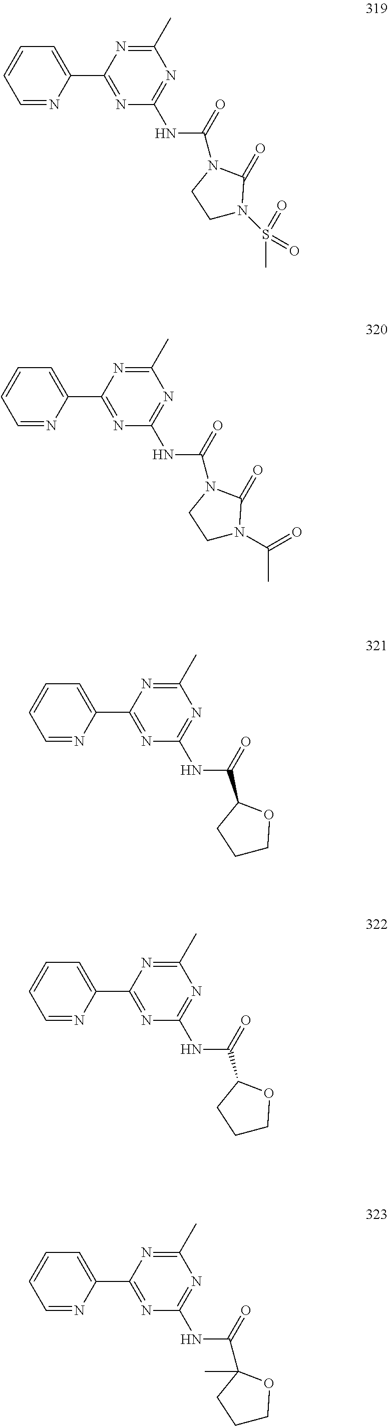

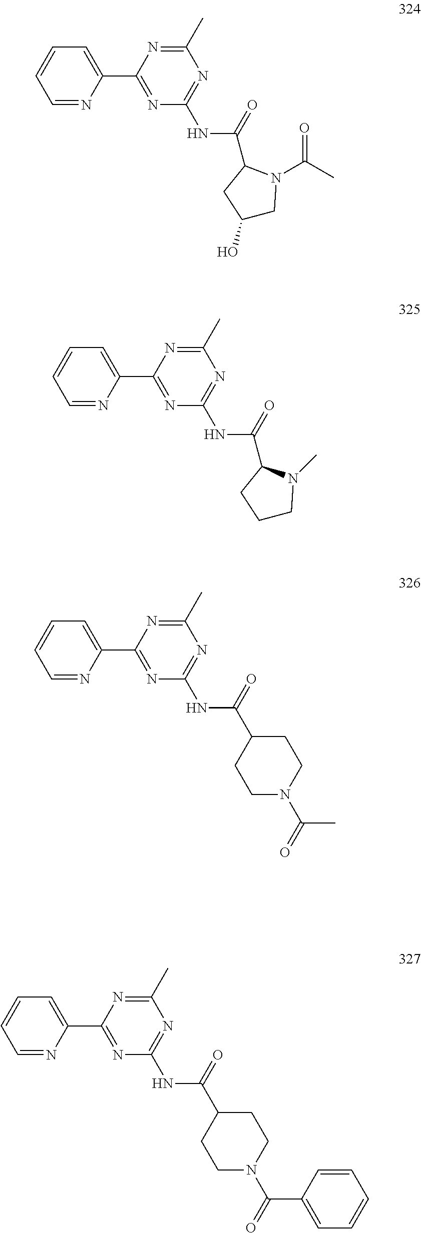

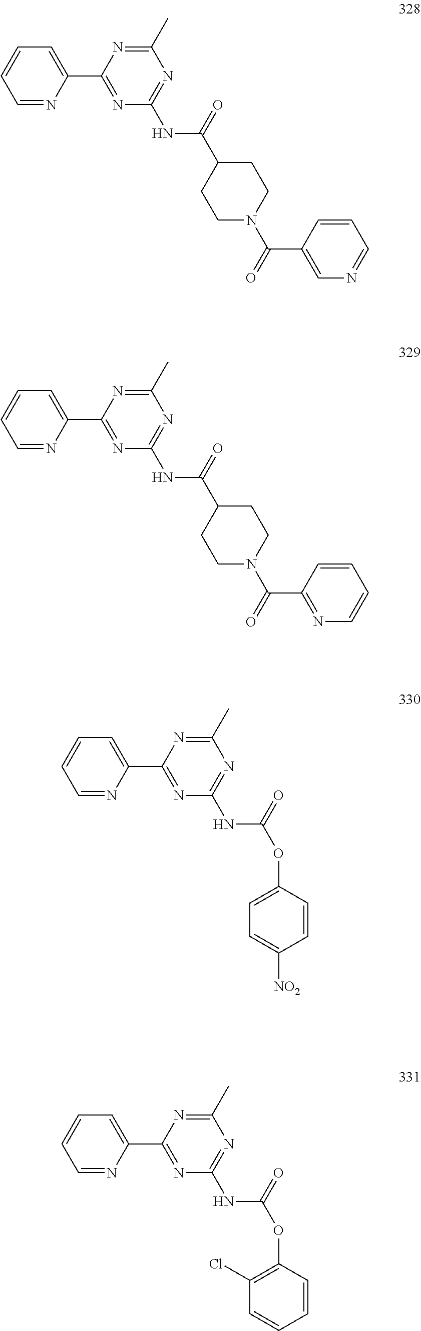

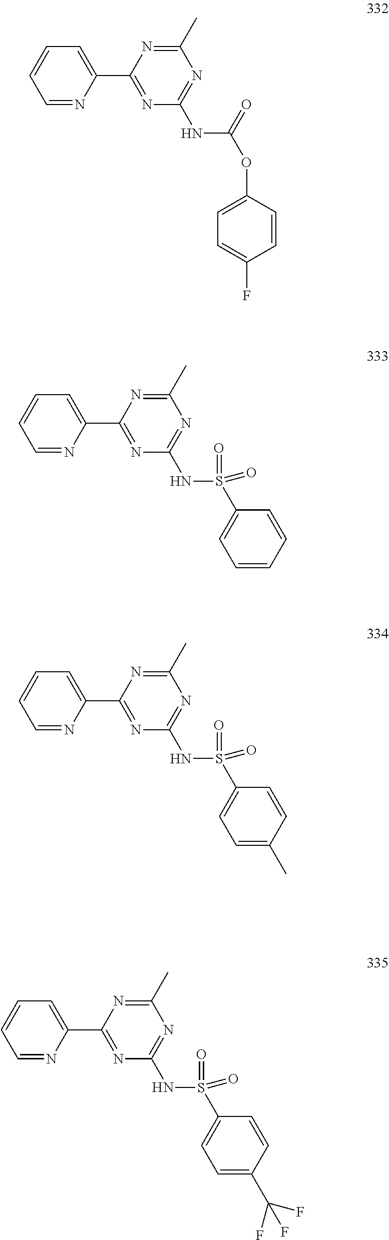

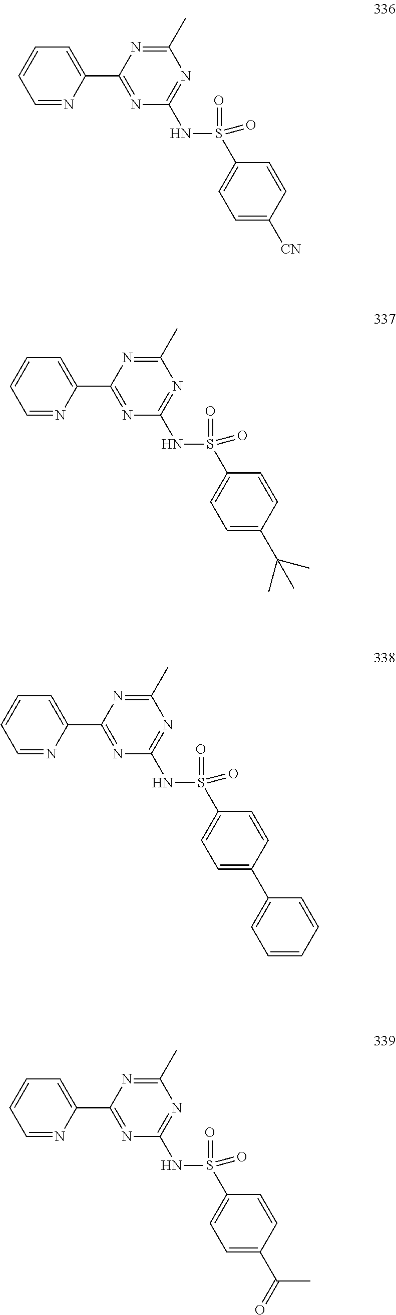

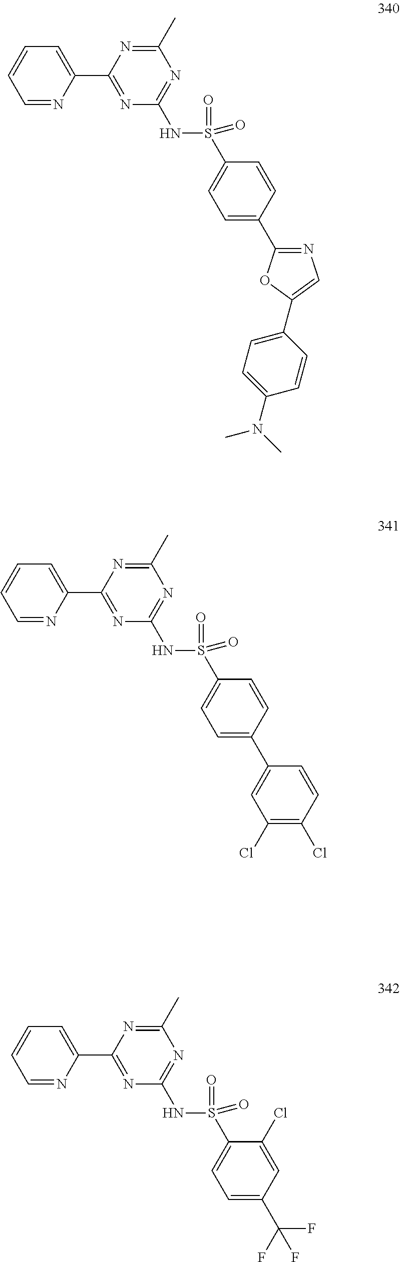

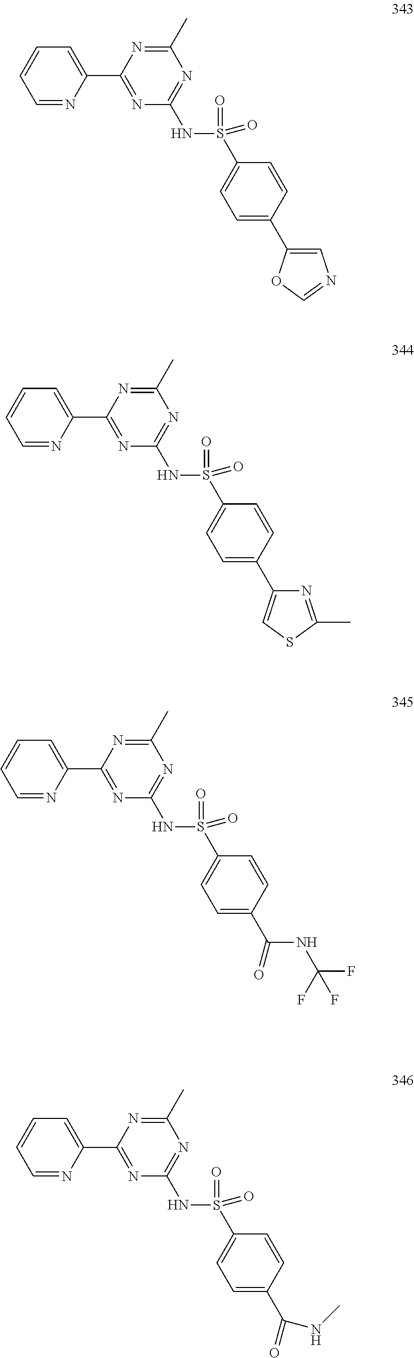

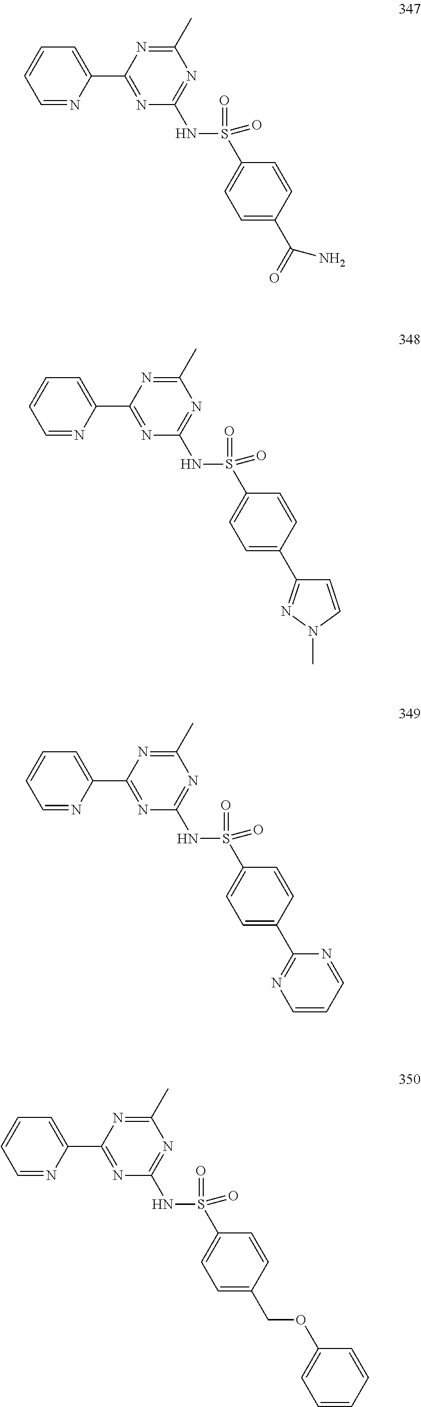

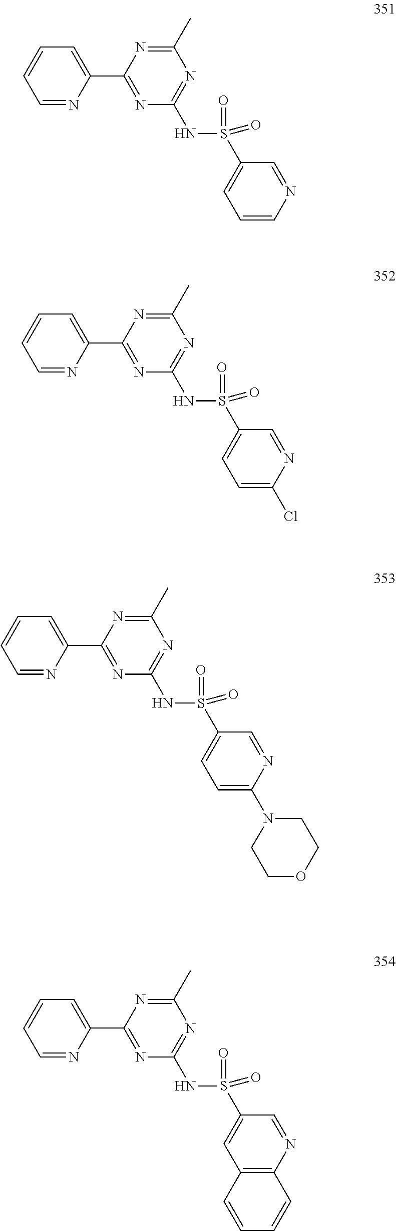

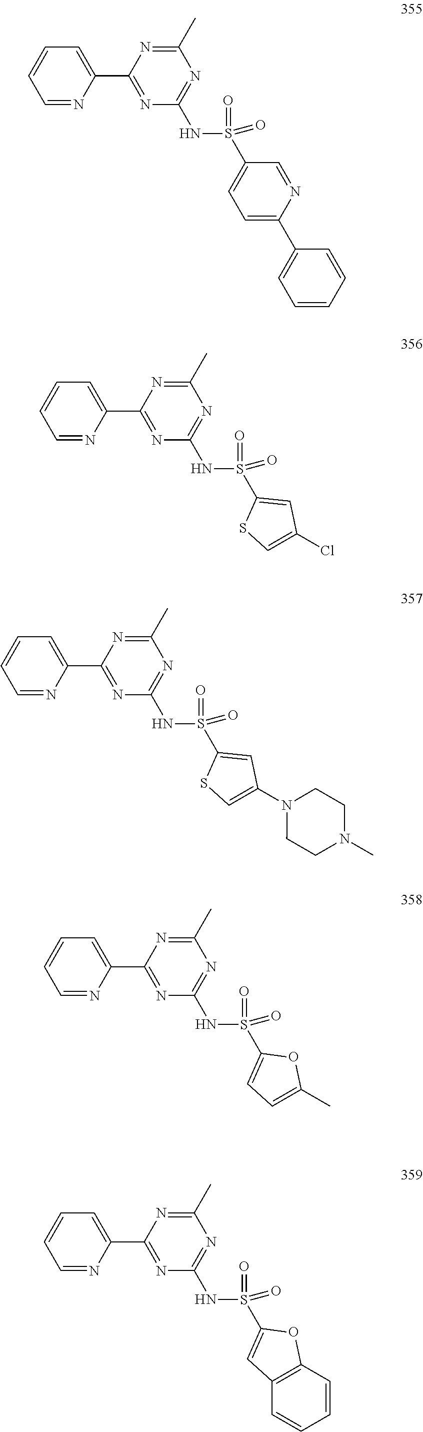

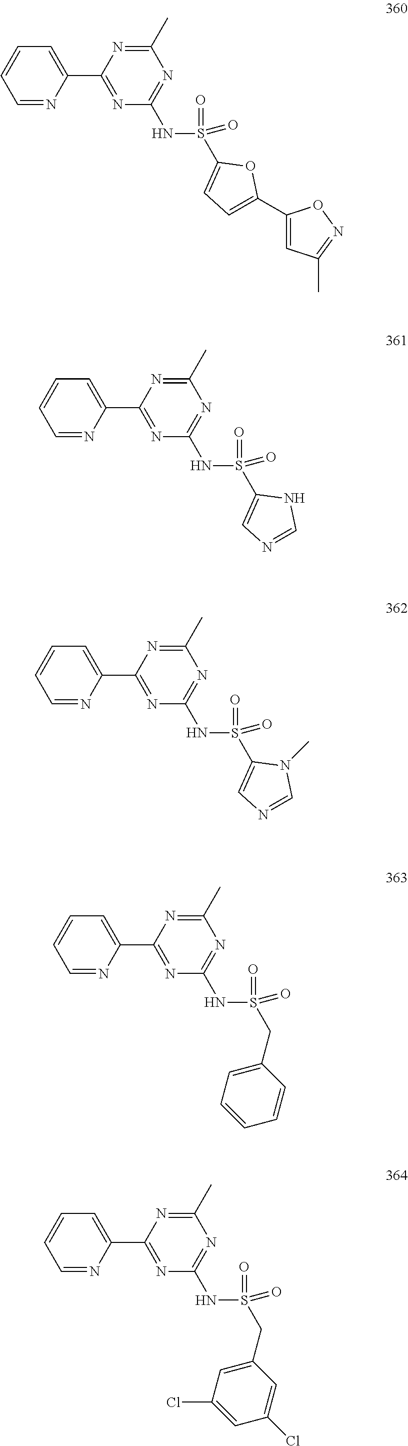

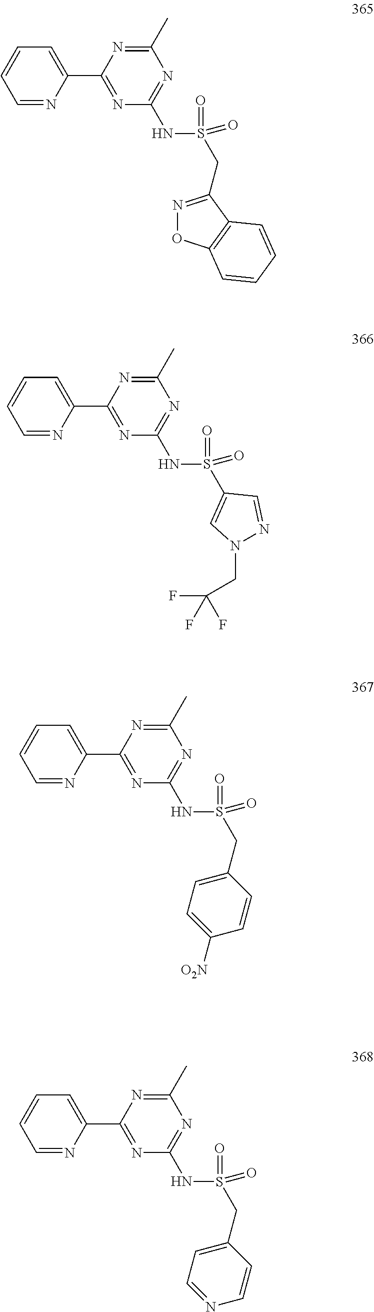

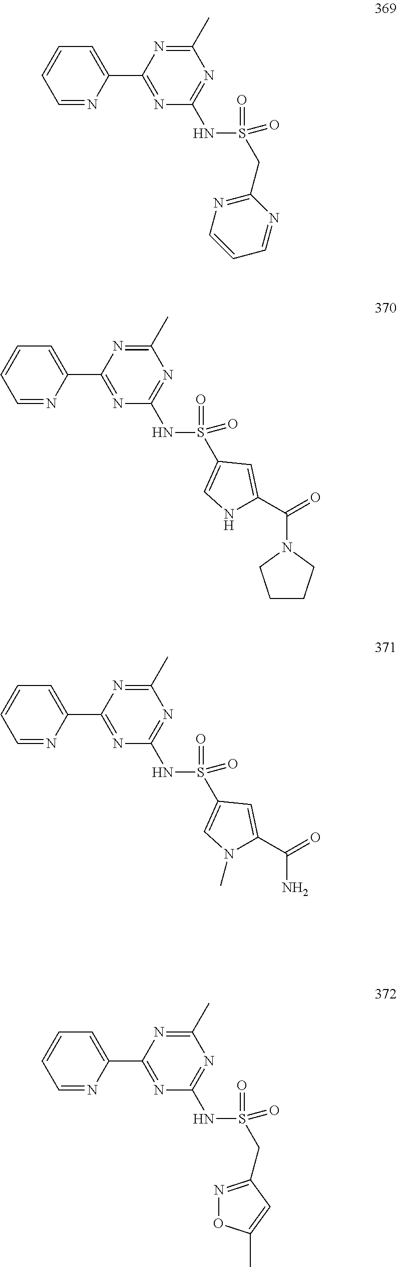

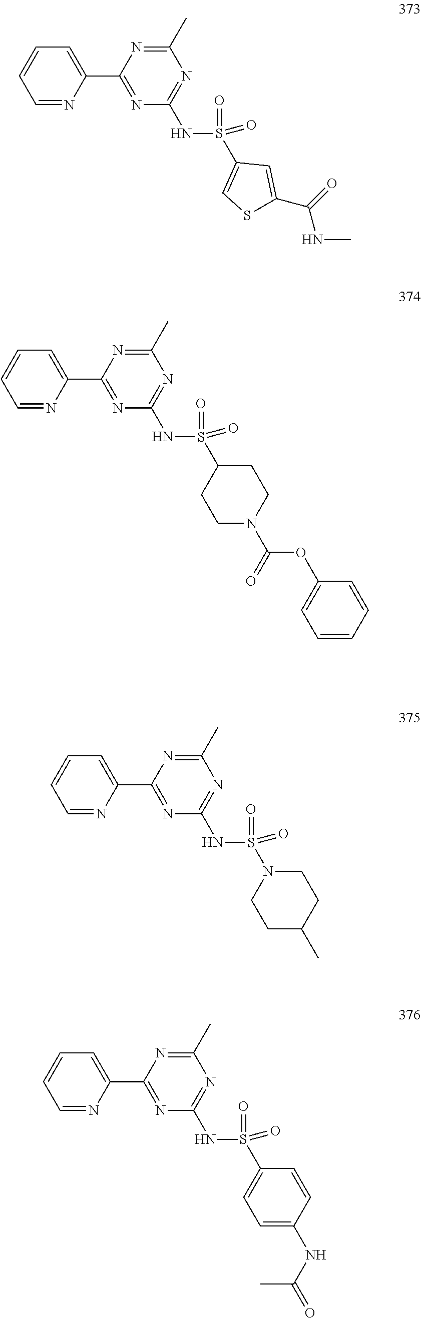

In another embodiment, a method of preparing the inventive compounds is provided. The compounds of the present invention can be generally prepared by coupling the central rings and A ring via established amide bond formation procedure. Compound (I) and (II) may contain various stereoisomers, geometric isomers, tautomeric isomers, and the like. All of possible isomers and their mixtures are included in the present invention, and the mixing ratio is not particularly limited.

Synthesis of the triazine of general formula (3) wherein R₁=methyl is preferably carried our as follows: first, pyridylamidines (1) known from the literature (for example, from Medwid, J. Med. Chem., 1990(33), 1230), where appropriate in the form of their salts, reacted with cyano-imidates, which have been prepared in accordance with data in the literature, for example as described in WO/0014056, or which are commercially available, preferably cyano-methyl-imidates (2), in an inert solvent, preferably methanol, to form the amino-methyl-triazine of formula (3). The latter compounds are then, in accordance with process known in international patent WO01/25220 A1, acylated with carboxylic acid, carboxylic anhydride and acid chloride (4), sulfamoyl halides (22) or isocyanate (5) to form the N-acyl-amino-triazine of formula (Ia and IIa, R₂═H) (Scheme 1). The reaction is advantageously carried out in an aprotic solvent in the presence of a base at ambient temperature.

The aprotic solvent of condensation reaction may be used, but not limited to, dichloromethane, acetone, dioxane, acetonitrile, chloroform, dichloroethane, diethyl ether, THF, DMF, and the like, or may be used alone or as a mixture thereof, conveniently at a temperature within the range −60° C. to reflux.

A variety of base agent may be used, but not limited to, pyridine, triethylamine, di-isopropylethylamine, methylamine, imidazole, benzimidazole, histidine, sodium hydride, and the like, preferally is pyridine, or may be used alone without solvents.

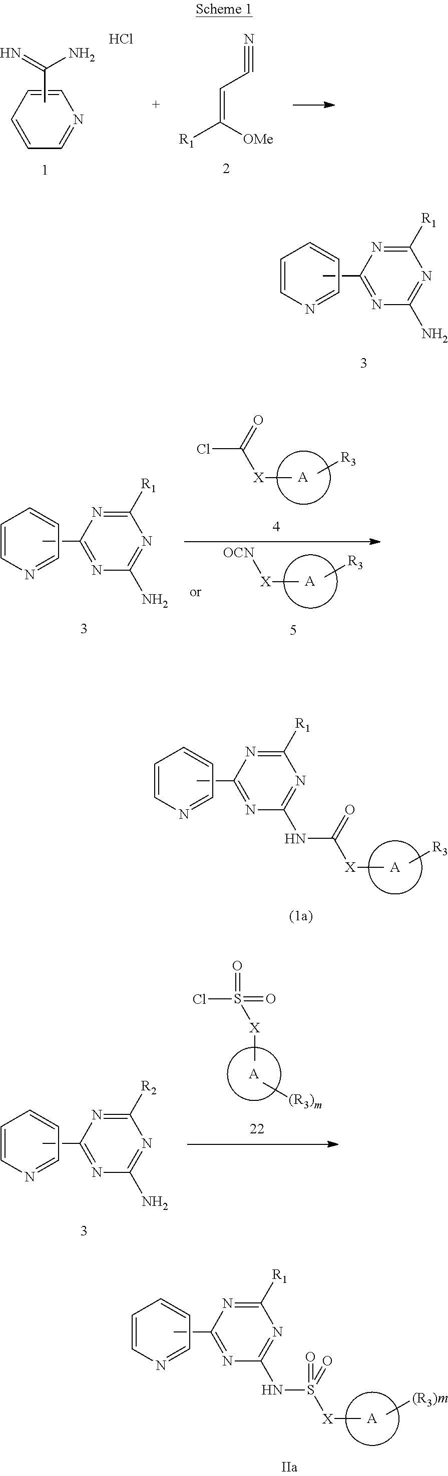

When the acid chloride (6) (wherein R′ represents the substituted or unsubstituted (C₁-C₁₀) alkyl or alkenyl) is alkyl acid chloride, the product 7 was subsequently reduced using reducing agents such as, for example, hydrogen, simple or complex metal hydrides, transition metals or salts thereof, but preferably using borane, to form the N-alkyl-amino-triazine of formula (8). The latter compounds are then, acylated with carboxylic acid, carboxylic anhydride and acid chloride or sulfonyl chloride to form the N-acyl-amino-triazine of formula (Ib and IIb, R₂=alkyl) (Scheme 2). The reaction is advantageously carried out in an aprotic solvent in the presence of a base at ambient temperature, and solvents and base have the same define as the preparation of formula Ia and IIa.

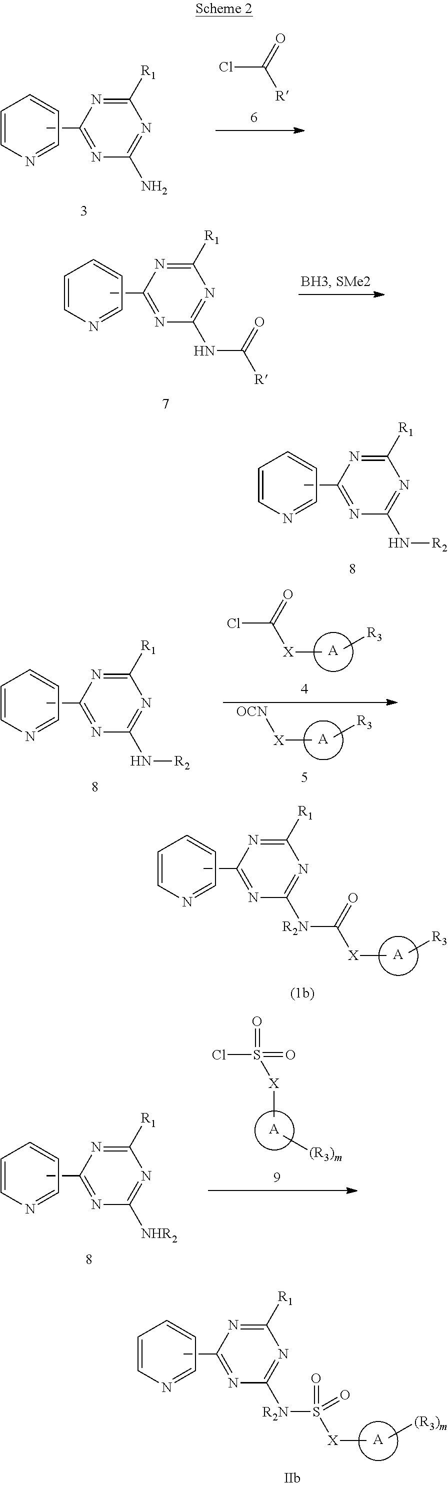

In scheme 3 that follows the term “reduction” referring to the process of reducing a nitro functionality to an amino functionality, the reduction of a nitro group can be carried out in a number of ways well known to those skilled in the art of organic synthesis including, but not limited to, catalytic hydrogenation, reduction with SnCl₂ and reduction with titanium bichloride. In a particular embodiment, the reduction reaction is performed at about 60° C. For an overview of reduction methods see: Hudlicky, M. Reductions in Organic Chemistry, ACS Monograph 188, 1996.

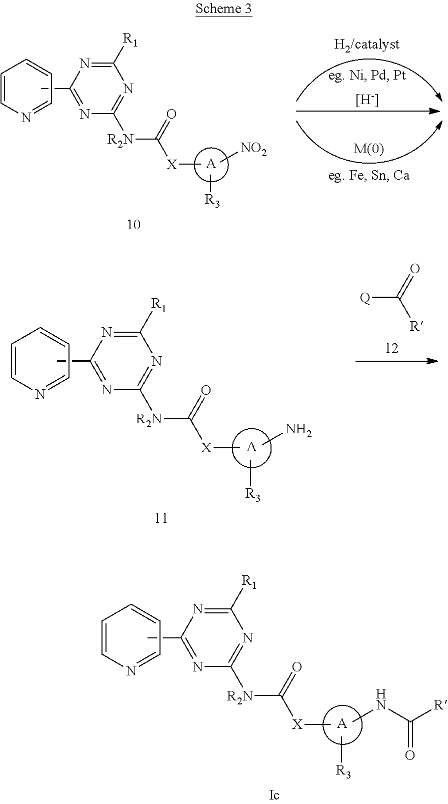

Intermediate 11 reacted with an activated acid (12) to yield final compound Ic. In a particular embodiment, the activated acid (12) (wherein R′ represents the substituted or unsubstituted (C₁-C₁₀) alkyl, alkenyl, (C3-C7) aryl or heteroaryl) is acid halide (for example Q is chloride) or activated ester (for example Q is O-EDC). In a particular embodiment the reaction is performed at about 0° C. to room temperature.

Intermediate 11 also reacted with the appropriate sulfonyl chloride R′—(SO₂) Cl (wherein R′ represents the substituted or unsubstituted (C₁-C₁₀) alkyl, alkenyl, (C3-C7) aryl or heteroaryl) in the presence of a non-nucleophilic base such as TEA or diisopropylethylamine to form the desired sulfonamide Id (Scheme 4).

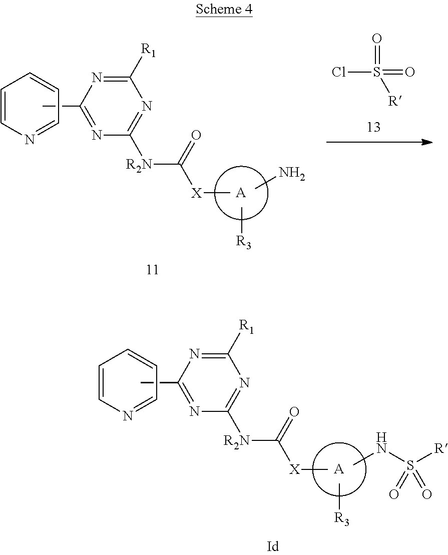

As illustrated in scheme 5, formula (Ie) can be prepared by condensation reaction with substituted aldehyde or ketone R₈COR₉ (wherein R₈ or R₉ independently or together represents hydrogen, the substituted or unsubstituted (C₁-C₁₀) alkyl, alkenyl, aryl or heteroaryl), and then the further reduction reaction.

The solvent of condensation reaction may be used, but not limited to, dichloromethane, acetone, dioxane, acetonitrile, chloroform, dichloroethane, diethyl ether, THF, DMF, and the like, or may be used alone or as a mixture thereof, conveniently at a temperature within the range −60° C. to room temperature.

A variety of reducing agent and reaction condition can be used to reduce imine. Sodium cyanoborohydride may be used as the reducing agent, and other reducing agents that can be used include, but are not limited to, sodium borohydride, sodium dithionite, lithium aluminum hydride, Red-Al, and the like. The solvent may be used, but not limited to, alcoholic solvents such as methanol and ethanol under neutral conditions at temperatures range from 0° C. to that of the refluxing solvent, DMF, acetonitrile, benzene, toluene, and the like.

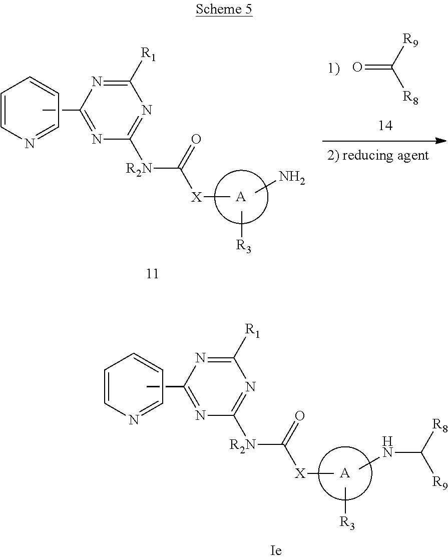

Triazine compound 15 with substituted heteroaryl with potential leaving groups (for example Q is Cl, Br, I, SO₂Me etc.) may undergo substitution reactions on treatment with nucleophiles, such as amine R₁₀NHR₁₁ (wherein R₁₀ or R₁₁ independently or together represents substituted or unsubstituted (C₁-C₁₀) alkyl, alkenyl, (C₃-C₇) aryl or heteroaryl) or secondary amine with formula 17 (wherein Y, R₆ and R₇ are as defined herein) to obtain compounds If and Ig (Scheme 6) in the presence of an inert solvent.

There is no particular restriction on the nature of the solvent to be employed, provided that it has no adverse effect on the reaction or on the reagents involved and that it can dissolve the reagents, at least to some extent. Examples of suitable solvents include: aromatic hydrocarbons, such as benzene, toluene and xylene; halogenated hydrocarbons, especially aromatic and aliphatic hydrocarbons, such as methylene chloride, chloroform, carbon tetrachloride, dichloroethane, chlorobenzene and the dichlorobenzenes; esters, such as ethyl formate, ethyl acetate, propyl acetate, butyl acetate and diethyl carbonate; ethers, such as diethyl ether, diisopropyl ether, tetrahydrofuran, dioxane. dimethoxyethane and diethylene glycol dimethyl ether; ketones, such as acetone, methyl ethyl ketone, methyl isobutyl ketone, isophorone and cyclohexanone; nitro compounds, which may be nitroalkanes or nitroaranes, such as nitroethane and nitrobenzene; nitriles, such as acetonitrile and isobutyronitrile; amides, which may be fatty acid amides, such as formamide, dimethylformamide, dimethylacetamide and hexamethylphosphoric triamide; and sulphoxides, such as dimethyl sulphoxide and sulpholane.

The reaction can take place over a wide range of temperatures, and the precise reaction temperature is not critical to the invention. In general, we find it convenient to carry out the reaction at a temperature of from −50° C. to 100° C.

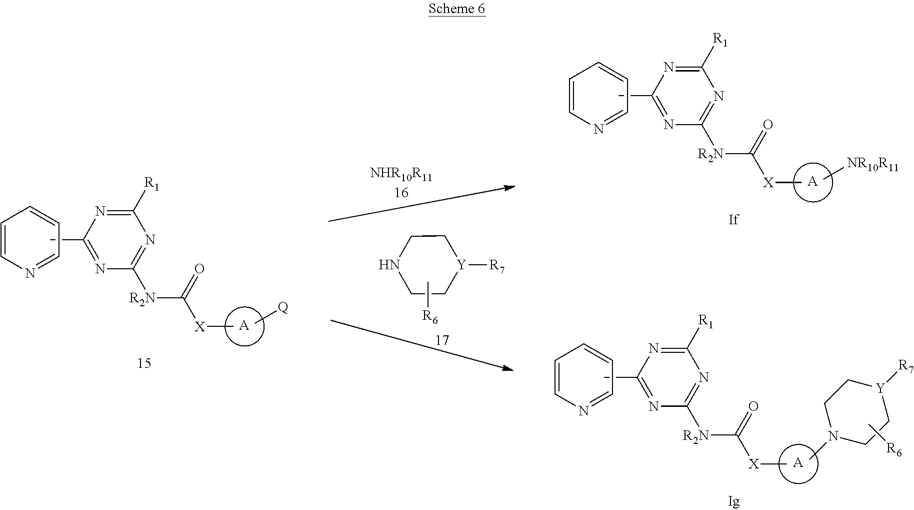

Scheme 7 illustrates one of the methods to swap the —OH group in the —COOH group of a carboxylic acid 16 for a chlorine atom to make acyl chlorides formula 4 (acid chlorides). A variety of chlorine agents and reaction conditions can be used. (Sulphur dichloride oxide (thionyl chloride) may be used as chlorine agent without solvent in the condition of refulx (Clayden, J., Organic chemistry. Oxford: Oxford University Press. 2001, 276-296), other agents that can be used include, but are not limited to, phosphorus (V) chloride, phosphorus (III) chloride (Boyd, R; Morrison, R., Organic chemistry, 1992, 666-762), oxalyl chloride and cyanuric chloride (Venkataraman, K. and Wagle, D. R, Tet. Lett. 1979, 20 (32): 3037-3040), and the like. Some HCl undurable acid can use Applye reaction to obtain the acyl chloride (Taschner, M. J., e-EROS: Encyclopedia of Reagents for Organic Synthesis, 2001).

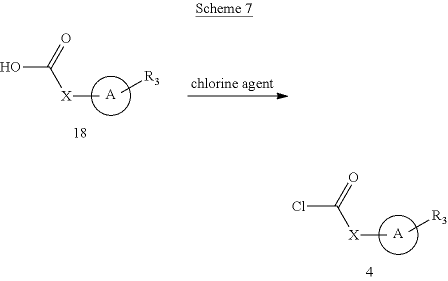

The preparation of the compound of formula (21, 22 or 23) in this invention can be carried out by methods known in the art (for example, Masquelin, T., Meunier, n., et al, Heterocycles, 1998. 48(12), 2489 and Masquelin, T., Delgado, Y., et al., Tetrahedron Letters, 1998, 39, 5725-5726). Commercially available methylthio-trazine-amine was oxidized to 2-methyllsulfonyl intermediate followed by reaction with nucleophiles, such as amine NHR₁₀R₁₁ (wherein R₁₀ and R₁₁ are defined herein), alhocol R₁₂OH (wherein R₁₂ represents substituted or unsubstituted (C₁-C₁₀) alkyl, alkenyl, (C₃-C₇) aryl or heteroaryl) and cyanide (for example sodium cyanide or potassium cyanide) to obtain pyridyl-amino-amine 21, pyridyl-ether-amine 22 and pyridyl-cyano-amine 23 (Scheme 7).

The oxidizing agent may be used, but not limited to, OXONE®, meta-chloroperbenzoic acid, peroxytrifluoroacetic acid, hydrogen peroxide. Suitable solvent can be, but not limited to chloroform, dichloromethane, benzene, toluene, or a mixture with an alcoholic solvent, such as methanol, ethanol, isopropanol, or 1-butanol, in particulat, ethanol. The oxidation reaction conveniently runs at a temperature within the range −60° C. to room temperature.

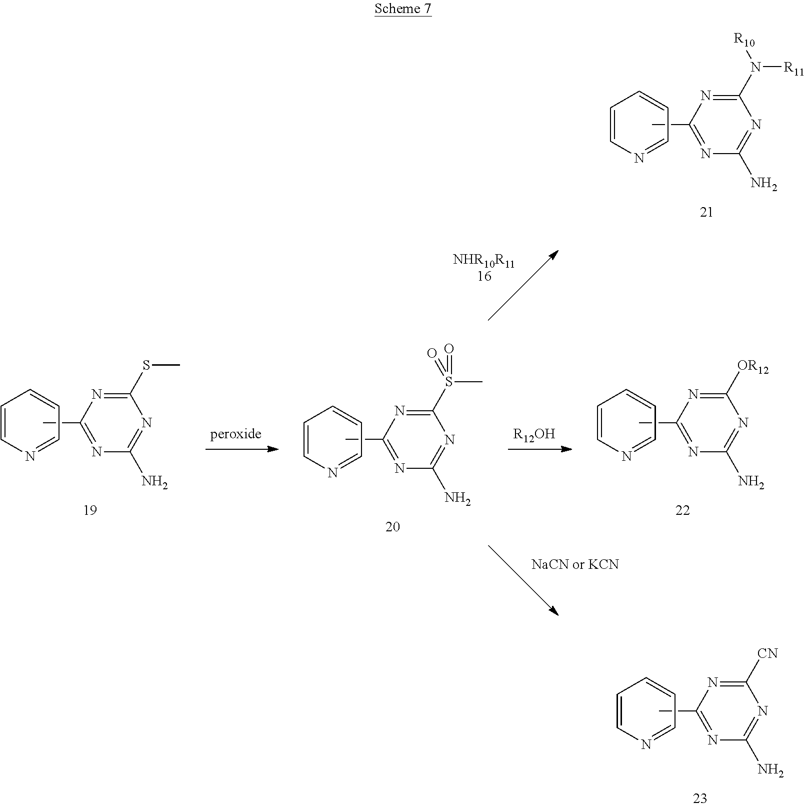

The present invention provides compositions of matter that are formulations of one or more active drugs and a pharmaceutically-acceptable carrier. In this regard, the invention provides a composition for administration to a mammalian subject, which may include a compound of formula I and II, or its pharmaceutically acceptable salts.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C1-4 alkyl)₄ salts. This invention also envisions the quaternization of any basic nitrogen-containing groups of the compounds disclosed herein. Water or oil-soluble or dispersible products may be obtained by such quaternization. The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutically acceptable compositions of this invention may be orally administered in any orally acceptable dosage form including, but not limited to, capsules, tablets, troches, elixirs, suspensions, syrups, wafers, chewing gums, aqueous suspensions or solutions.

The oral compositions may contain additional ingredients such as: a binder such as microcrystalline cellulose, gum tragacanth or gelatin; an excipient such as starch or lactose, a disintegrating agent such as alginic acid, corn starch and the like; a lubricant such as magnesium stearate; a glidant such as colloidal silicon dioxide; and a sweetening agent such as sucrose or saccharin or flavoring agent such as peppermint, methyl salicylate, or orange flavoring. When the dosage unit form is a capsule, it may additionally contain a liquid carrier such as a fatty oil. Other dosage unit forms may contain other various materials which modify the physical form of the dosage unit, such as, for example, a coating. Thus, tablets or pills may be coated with sugar, shellac, or other enteric coating agents. A syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days. Alternate day dosing or dosing once every several days may also be utilized.

Sterile, injectable solutions may be prepared by incorporating a compound in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

In accordance with the invention, there are provided compositions containing triazine derivatives and methods useful for the in vivo delivery of triazine derivatives in the form of nanoparticles, which are suitable for any of the aforesaid routes of administration.

U.S. Pat. Nos. 5,916,596, 6,506,405 and 6,537,579 teach the preparation of nanoparticles from the biocompatible polymers, such as albumin. Thus, in accordance with the present invention, there are provided methods for the formation of nanoparticles of the present invention by a solvent evaporation technique from an oil-in-water emulsion prepared under conditions of high shear forces (e.g., sonication, high pressure homogenization, or the like).

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

In accordance with the invention, the compounds of the invention inhibit the hedgehog signaling and may be used to treat cancers associated with aberrant hedgehog signaling, cellular proliferation or hyperproliferation, such as cancers which include but are not limited to tumors of the nasal cavity, paranasal sinuses, nasopharynx, oral cavity, oropharynx, larynx, hypopharynx, salivary glands, and paragangliomas. The compounds of the invention may also be used to treat cancers of the liver and biliary tree (particularly hepatocellular carcinoma), intestinal cancers, particularly colorectal cancer, ovarian cancer, small cell and non-small cell lung cancer, breast cancer, sarcomas (including fibrosarcoma, malignant fibrous histiocytoma, embryonal rhabdomysocarcoma, leiomysosarcoma, neuro-fibrosarcoma, osteosarcoma, synovial sarcoma, liposarcoma, and alveolar soft part sarcoma), neoplasms of the central nervous systems (particularly brain cancer), and lymphomas (including Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma).

The compounds and methods of the present invention, either when administered alone or in combination with other agents (e.g., chemotherapeutic agents or protein therapeutic agents described below) are also useful in treating a variety of disorders, including but not limited to, for example: stroke, cardiovascular disease, myocardial infarction, congestive heart failure, cardiomyopathy, myocarditis, ischemic heart disease, coronary artery disease, cardiogenic shock, vascular shock, pulmonary hypertension, pulmonary edema (including cardiogenic pulmonary edema), pleural effusions, rheumatoid arthritis, diabetic retinopathy, retinitis pigmentosa, and retinopathies, including diabetic retinopathy and retinopathy of prematurity, inflammatory diseases, restenosis, asthma, acute or adult respiratory distress syndrome (ARDS), lupus, vascular leakage, protection from ischemic or reperfusion injury such as ischemic or reperfusion injury incurred during organ transplantation, transplantation tolerance induction; ischemic or reperfusion injury following angioplasty; arthritis (such as rheumatoid arthritis, psoriatic arthritis or osteoarthritis); multiple sclerosis; inflammatory bowel disease, including ulcerative colitis and Crohn's disease; lupus (systemic lupus crythematosis); graft vs. host diseases; T-cell mediated hypersensitivity diseases, including contact hypersensitivity, delayed-type hypersensitivity, and gluten-sensitive enteropathy (Celiac disease); Type 1 diabetes; psoriasis; contact dermatitis (including that due to poison ivy); Hashimoto's thyroiditis; Sjogren's syndrome; Autoimmune Hyperthyroidism, such as Graves' disease; Addison's disease (autoimmune disease of the adrenal glands); autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; cancers, including those where kineses such as Src-family kineses are activated or overexpressed, such as colon carcinoma and thymoma, or cancers where kinase activity facilitates tumor growth or survival; glomerulonephritis, serum sickness; uticaria; allergic diseases such as respiratory allergies (asthma, hayfever, allergic rhinitis) or skin allergies; mycosis fungoides; acute inflammatory responses (such as acute or adult respiratory distress syndrome and ischemialreperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic schlerosis; morphea; peripheral limb ischemia and ischemic limb disease; bone disease such as osteoporosis, osteomalacia, hyperparathyroidism, Paget's disease, and renal osteodystrophy; vascular leak syndromes, including vascular leak syndromes induced by chemotherapies or immunomodulators such as IL-2; spinal cord and brain injury or trauma; glaucoma; retinal diseases, including macular degeneration; vitreoretinal disease; pancreatitis; vasculatides, including vasculitis, Kawasaki disease, thromboangiitis obliterans, Wegener s granulomatosis, and Behcet's disease; scleroderma; preeclampsia; thalassemia; Kaposi's sarcoma; von Hippel Lindau disease; and the like.

The invention also provides methods of treating a mammal afflicted with the above diseases and conditions. The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

In one aspect, the invention compounds are administered in combination with chemotherapeutic agent, an anti-inflammatory agent, antihistamines, chemotherapeutic agent, immunomodulator, therapeutic antibody or a protein kinase inhibitor, e.g., a tyrosine kinase inhibitor, to a subject in need of such treatment.

The method includes administering one or more of the inventive compounds to the afflicted mammal. The method may further include the administration of a second active agent, such as a cytotoxic agent, including alkylating agents, tumor necrosis factors, intercalators, microtubulin inhibitors, and topoisomerase inhibitors. The second active agent may be co-administered in the same composition or in a second composition. Examples of suitable second active agents include, but are not limited to, a cytotoxic drug such as Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-□a; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

In accordance with the invention, the compounds and compositions may be used at sub-cytotoxic levels in combination with other agents in order to achieve highly selective activity in the treatment of non-neoplastic disorders, such as heart disease, stroke and neurodegenerative diseases (Whitesell et al., Curr Cancer Drug Targets (2003), 3(5), 349-58).

The exemplary therapeutical agents that may be administered in combination with invention compounds include EGFR inhibitors, such as gefitinib, erlotinib, and cetuximab. Her2 inhibitors include canertinib, EKB-569, and GW-572016. Also included are Src inhibitors, dasatinib, as well as Casodex (bicalutamide), Tamoxifen, MEK-1 kinase inhibitors, MARK kinase inhibitors, PI3 inhibitors, and PDGF inhibitors, such as imatinib, Hsp90 inhibitors, such as 17-AAG and 17-DMAG. Also included are anti-angiogenic and antivascular agents which, by interrupting blood flow to solid tumors, render cancer cells quiescent by depriving them of nutrition. Castration, which also renders androgen dependent carcinomas non-proliferative, may also be utilized. Also included are IGF1R inhibitors, inhibitors of non-receptor and receptor tyrosine kineses, and inhibitors of integrin.

The pharmaceutical composition and method of the present invention may further combine other protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay. The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

Other therapeutic agents for the combinatory therapy include cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and for gpn39 (i.e., CD154), fusion proteins constructed from CD40 and gp39 (CD40lg and CD8gp39), inhibitors, such as nuclear translocation inhibitors, of NF-kappa B function, such as deoxyspergualin (DSG), cholesterol biosynthesis inhibitors such as HM:G CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-a inhibitors such as tenidap, anti-TNF antibodies or soluble TNF receptor, and rapamycin (sirolimus or Rapamune) or derivatives thereof.

When other therapeutic agents are employed in combination with the compounds of the present invention they may be used for example in amounts as noted in the Physician Desk Reference (PDR) or as otherwise determined by one having ordinary skill in the art.

### EXAMPLES

The following examples are provided to further illustrate the present invention but, of course, should not be construed as in any way limiting its scope

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO₃) and sodium chloride (brine) were saturated.

Analytical thin layer chromatography (TLC) was carried out on Merck Kiesel gel 60 F254 plates with visualization by ultraviolet and/or anisaldehyde, potassium permanganate or phosphomolybdic acid dips.

NMR spectra: 1H Nuclear magnetic resonance spectra were recorded at 400 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken and calculated directly from the spectra and are uncorrected.

LC/mass spectra: Electrospray (ES+) ionization was used. The protonated parent ion (M+H) or parent sodium ion (M+Na) or fragment of highest mass is quoted. Analytical gradient consisted of 10% ACN in water ramping up to 100% ACN over 5 minutes unless otherwise stated.

### Example 1

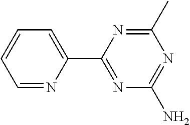

A solution of sodium methoxide (350 mg, 6.4 mmol) in 5 mL methanol is added dropwise, at room temperature, to a solution of pyrid-2-ylamide hydrochloride (628 mg, 6.4 mmol) in 5 mL of methanol. After stirring for 20 min, the solution is then added dropwise to methyl-N-cyanoethanimidate, with stirring. After stirring for 12 hours at room temperature, removed the solvents and purified on the flash chromatography system to obtain compound 1 (600 mg, 50% yield) as a white solid. ¹H NMR (400 MHz, DMSO-d⁶): δ 8.70-8.69 (m, 1H), 8.28-8.26 (m, 1H), 7.96-7.91 (m, 1H), 7.62 (brs, 1H), 7.54-7.51 (m, 2H), 2.37 (s, 3H). MS (ESI): Calcd. for C₉H₉N₅: 187, found 188 (M+H)⁺

### Example 2

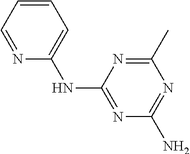

Preparation: 2-methyl-1,3,5-triazine-4,6-dicloride (368 mg, 2.24 mmol) was dissolved into 5 mL DMF, and then added the 3 mL DMF solution of 2-pyridylamine (211 mg, 2.24 mmol) and DIPEA (0.47 mL, 2.69 mmol) at 0 C. Slowly increased the temperature to room temperature and stirred at rt for 3 hours, and then added the 3 mL DMF solution of NH3.H2O (314 mg, 25% water solution) and DIPEA (0.47 mL, 2.69 mmol) at room temperature. The mixture was stirred at room temperature for overnight, and then removed the solvents and purified on the flash chromatography system to obtain compound 2 (52.1 mg, 11% yield) as a white solid. ¹H NMR (400 MHz, DMSO-d⁶): δ 9.45 (s, 1H), 8.36 (d, J=8.4 Hz, 1H), 8.28-8.26 (s, 1H), 7.74-7.70 (m, 1H), 7.10 (brs, 2H), 7.02-6.98 (m, 1H), 2.22 (s, 3H). MS (ESI): Calcd. for C₉H₁₀N₆: 202, found 203 (M+H)⁺

### Example 3

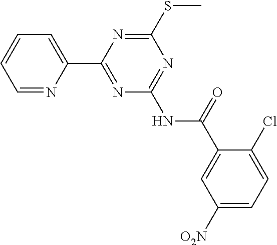

Preparation: 4-(methylthio)-6-(pyridin-2-yl)-1,3,5-triazin-2-amine (100 mg, 0.46 mmol) was dissolved into 5 mL pyridine, and then added 2-chloro-5-nitrobenzoyl chloride (100.3 mg, 0.46 mmol) at room temperature. Increased the temperature to 100 C and then stirred at that temperature for overnight, removed the solvents and purified on the flash chromatography system to obtain compound 3 (90 mg, 49% yield) as a white solid. ¹H NMR (400 MHz, DMSO-d⁶): δ 12.06 (s, 1H), 8.73-8.72 (m, 1H), 8.48 (dd, J=0.4 and 2.8 Hz, 1H), 8.33 (dd, J=2.8 and 8.8 Hz, 1H), 8.13 (d, J=7.6 Hz, 1H), 7.98-7.94 (m, 1H), 7.85 (dd, J=0.4 and 8.8 Hz, 1H), 7.63-7.59 (m, 1H), 2.40 (s, 3H). MS (ESI): Calcd. for C₁₆H₁₁N₆ClO₃S: 402, found 403 (M+H)⁺

### Example 4

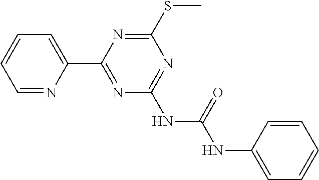

Preparation: 4-(methylthio)-6-(pyridin-2-yl)-1,3,5-triazin-2-amine (50 mg, 0.23 mmol) was dissolved into 2 mL pyridine, and then added isocyanatobenzene (27 mg, 0.23 mmol) at room temperature. Increased the temperature to 100 C and then stirred at that temperature for overnight, removed the solvents and purified on the flash chromatography system to obtain compound 4 (30 mg, 39% yield) as a white solid. ¹H NMR (400 MHz, DMSO-d⁶): δ 12.30 (s, 1H), 10.75 (s, 1H), 8.96-8.94 (m, 1H), 8.53-8.50 (m, 1H), 8.11-8.07 (m, 1H), 7.79-7.77 (m, 2H), 7.74-7.70 (m, 1H), 7.43-7.39 (m, 2H), 7.12-7.08 (m, 1H), 2.66 (s, 3H). MS (ESI): Calcd. for C₁₆H₁₄N₆O: 338, found 339 (M+H)⁺

### Example 5

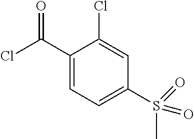

Preparation: 2-chloro-4-(methylsulfonyl)benzoic acid (62 mg, 0.26 mmol) was added into 3 mL SOCl2, and then reflux for 3 h, removed the solvents to obtain white solid. Dried on vacuum for 30 min, and then used at next step without purification.

### Example 6

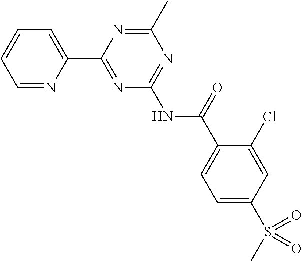

Preparation: 4-methyl-6-(pyridin-2-yl)-1,3,5-triazin-2-amine compound 1 (50 mg, 0.26 mmol) was dissolved into 2 mL pyridine, and then added HH328 at room temperature. Increased the temperature to 100 C and then stirred at that temperature for overnight, removed the solvents and purified on the flash chromatography system to obtain compound 6 (15 mg, 14% yield) as a white solid. ¹H NMR (400 MHz, DMSO-d⁶): δ 12.01 (s, 1H), 8.72 (s, 1H), 8.09 (dd, J=0.4 and 2.0 Hz, 1H), 7.99 (dd, J=1.6 and 8.0 Hz, 1H), 7.91-7.86 (m, 1H), 7.83 (d, J=8.0 Hz, 1H), 7.73-7.72 (m, 1H), 7.60-7.58 (m, 1H), 2.54 (s, 3H). MS (ESI): Calcd. for C17H14ClN5O₃S: 403, found 404 (M+H)⁺

### Example 7

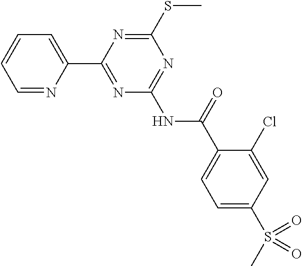

4-(methylthio)-6-(pyridin-2-yl)-1,3,5-triazin-2-amine (50 mg, 0.23 mmol) was dissolved into 2 mL pyridine, and then added HH328 at room temperature. Increased the temperature to 100 C and then stirred at that temperature for overnight, removed the solvents and purified on the flash chromatography system to obtain compound 7 (35 mg, 30% yield) as a white solid. ¹H NMR (400 MHz, DMSO-d⁶): δ 12.06 (s, 1H), 8.75-8.73 (m, 1H), 8.10 (dd, J=0.4 and 1.6 Hz, 1H), 7.99 (dd, J=2.0 and 8.0 Hz, 1H), 7.93-7.92 (m, 2H), 7.85 (dd, J=0.4 and 8.0 Hz, 1H), 7.62-7.59 (m, 1H), 3.34 (s, 3H), 2.32 (s, 3H). MS (ESI): Calcd. for C17H14ClN5O₃S₂: 435, found 436 (M+H)⁺

### Example 8

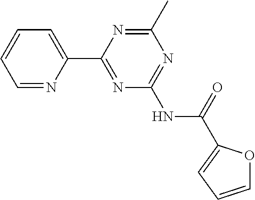

4-methyl-6-(pyridin-2-yl)-1,3,5-triazin-2-amine (50 mg, 0.26 mmol) was dissolved into 2 mL pyridine, and then added furan-2-carbonyl chloride (52 mg, 0.40 mmol) at room temperature. Increased the temperature to 100 C and then stirred at that temperature for overnight, removed the solvents and purified on the flash chromatography system to obtain compound 8 (10 mg, 13% yield) as a red solid. ¹H NMR (400 MHz, DMSO-d⁶): δ 11.34 (s, 1H), 8.80-8.79 (m, 1H), 8.46-8.44 (m, 1H), 8.06-8.02 (m, 1H), 8.00 (dd, J=0.8 and 1.6 Hz, 1H), 7.68 (dd, J=0.8 and 1.6 Hz, 1H), 7.65-7.62 (m, 1H), 6.73 (dd, J=1.6 and 3.6 Hz, 1H), 2.64 (s, 3H). MS (ESI): Calcd. for C14H11N5O2: 281, found 282 (M+H)⁺

### Example 9

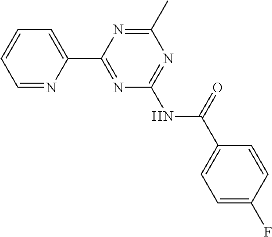

4-methyl-6-(pyridin-2-yl)-1,3,5-triazin-2-amine (50 mg, 0.26 mmol) was dissolved into 2 mL pyridine, and then added 4-fluorobenzoyl chloride (63 mg, 0.40 mmol) at room temperature. Increased the temperature to 100 C and then stirred at that temperature for overnight, removed the solvents and purified on the flash chromatography system to obtain compound 9 (10 mg, 13% yield) as a white solid. ¹H NMR (400 MHz, DMSO-d⁶): δ 11.56 (brs, 1H), 8.80-8.78 (m, 1H), 8.43-8.41 (m, 1H), 8.09-8.01 (m, 3H), 7.64-7.61 (m, 1H), 7.38-7.33 (m, 2H), 2.64 (s, 3H). MS (ESI): Calcd. for C16H12FN5O: 309, found 310 (M+H)⁺

### Example 10

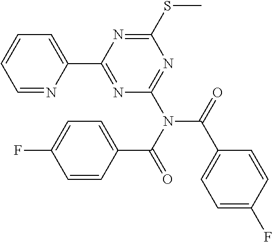

4-(methylthio)-6-(pyridin-2-yl)-1,3,5-triazin-2-amine (50 mg, 0.23 mmol) was dissolved into 2 mL pyridine, and then added 4-fluorobenzoyl chloride (54 mg, 0.34 mmol) at room temperature. Stirred at room temperature for 2 days, removed the solvents and purified on the flash chromatography system to obtain compound 10 (50 mg, 47% yield) as a white solid. ¹H NMR (400 MHz, DMSO-d⁶): δ 8.76-8.74 (m, 1H), 8.02-7.93 (m, 6H), 7.63-7.59 (m, 1H), 7.41-7.37 (m, 4H), 2.22 (s, 3H). MS (ESI): Calcd. for C23H15F2N5O2S: 463, found 464 (M+H)⁺

### Example 11

### Hedgehog Signalling Inhibition Assays

Hh-dependent C3H10T1/2 differentiation assay: C3H10T1/2 cells are multipotent mesenchymal progenitor cells that have the potential to differentiate into osteoblasts upon stimulation of the Hh pathway. Osteoblasts produce substantial alkaline phosphatase (AP) that can easily be measured with an enzymatic assay. Briefly, mouse embryonic mesoderm fibroblasts C3H10T1/2 cells (obtained from ATCC Cat# CCL-226) were cultured in Basal MEM Media (Gibco/Invitrogen) supplemented with 10% heat inactivated FBS (Hyclone), 50 units/ml penicillin, 50 μg/ml streptomycin (Gibco/Invitrogen) and 2 mM Glutamine (Gibco/Invitrogen) at 37° C. with 5% CO2 in air atmosphere. Cells were dissociated with 0.05% trypsin and 0.02% EDTA in PBS for passage and plating. C3H10T1/2 cells were plated in 96 wells with a density of 8×103 cells/well. Cells were grown to confluence (72 h). Media containing 5 μM of 20(S)-hydroxycholesterol and 5 μM of 22(S)-hydroxycholesterol and/or compound was added at the start of the assay and left for 72 h. Media was aspirated and cells were washed once in PBS. Alkaline phosphatase was measured by adding 100 μL Tropix CDP-Star with Emerald II (0.4 mM Cat # MS100RY) to the well and the plate was incubated at room temperature in the dark for one hour. The plates were read on an Envision plate reader at 405 nm. The percent inhibition with respect to compound concentration was plotted using Prism graphing software on a semi-log plot and EC50s determined by non-linear regression analysis with a four-parameter logistic equation.

What is claimed is:

1. A compound of the formula 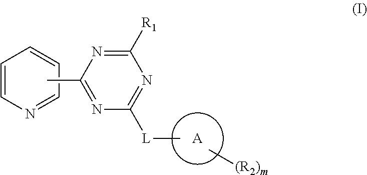
or a pharmaceutically acceptable salt thereof, wherein:
L is NR₃CO, NR₃SO₂, NR₃CONH, NR₃CSNH or NR₃CHR₄;
R₁ is selected from:
(viii) amino, alkyl amino, aryl amino, heteroaryl amino;
(ix) alkylthio, sulfinyl, sulfonyl, sulfamoyl;
(x) alkyloxy, Alkanoyl, alkoxycarbonyl;
(xi) hydrogen, C₁-C₆ alkyl, cycloalkyl, C₂-C₆ alkenyl, C₂-C₆ alkynyl;
(xii) aryl, heterocyclic, heteroaryl;
(xiii) C₁-C₆ trifluoroalkyl, cyano and;
(xiv) groups of the formula (a): 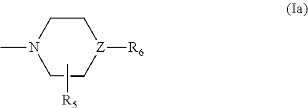
wherein:
R₅ represents hydrogen, C₁-C₄ alkyl, oxo;
Z is CH, when R₆ is hydrogen; or Z—R₆ is O; or Z is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁C₆alkyl)aminocarbonyl, each of which is substituted with from 0 to 4 substituents independently chosen from halogen, hydroxy, cyano, amino, —COOH and oxo;
ring A is aryl, heterocyclic, heteroaryl;
R₂ is hydroxyl, halogen, amino, nitro, cyano, alkyl, alkenyl, alkynyl, alkanoyl, alkylthio, sulfonyl, sulfinyl, alkoxy, alkoxycarbonyl, carbamoyl, acylamine, sulfamoyl or sulfonamide;
or R₂ is a aryl, heterocyclic or heteroaryl that is optionally substituted with hydroxyl, halogen, amino, nitro, cyano, alkyl, acyl, sulfonyl, sulfinyl, alkoxy, carbamoyl, acylamine, sulfamoyl and sulfonamide;
R₃ and R₄ are independently selected from hydrogen or an optionally substituted C₁₋₄ alkyl group; and
m is 0-4.

2. A compound of the formula 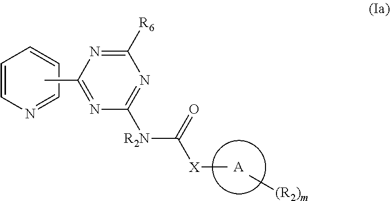
or a pharmaceutically acceptable salt thereof, wherein
R₁ is selected from:
(viii) amino, alkyl amino, aryl amino, heteroaryl amino;
(ix) alkylthio, sulfinyl, sulfonyl, sulfamoyl;
(xv) alkyloxy, Alkanoyl, alkoxycarbonyl;
(xvi) hydrogen, C₁-C₆ alkyl, cycloalkyl, C₂-C₆ alkenyl, C₂-C₆ alkynyl;
(xvii) aryl, heterocyclic, heteroaryl;
(xviii) C₁-C₆ trifluoroalkyl, cyano and;
(xix) groups of the formula (a): 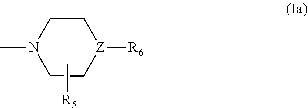
wherein:
R₅ represents hydrogen, C₁-C₄ alkyl, oxo;
Z is CH, when R₆ is hydrogen; or Z—R₆ is O; or Z is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁C₆alkyl)aminocarbonyl, each of which is substituted with from 0 to 4 substituents independently chosen from halogen, hydroxy, cyano, amino, —COOH and oxo;
ring A is aryl, heterocyclic, heteroaryl;
R₂ is hydroxyl, halogen, amino, nitro, cyano, alkyl, alkenyl, alkynyl, alkanoyl, alkylthio, sulfonyl, sulfinyl, alkoxy, alkoxycarbonyl, carbamoyl, acylamine, sulfamoyl or sulfonamide;
or R₂ is a aryl, heterocyclic or heteroaryl that is optionally substituted with hydroxyl, halogen, amino, nitro, cyano, alkyl, acyl, sulfonyl, sulfinyl, alkoxy, carbamoyl, acylamine, sulfamoyl and sulfonamide;
R₃ and R₄ are independently selected from hydrogen or an optionally substituted C1-4 alkyl group;
m is 0-4;
X is absent, O, CR₄R₇ or NR₃; and
R₇ is hydrogen or an optionally substituted C₁-C₄ alkyl group.

3. A compound of the formula 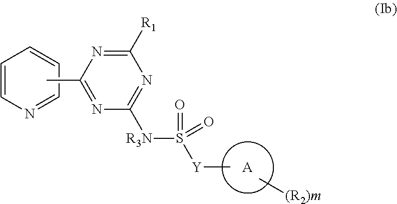
or a pharmaceutically acceptable salt thereof, wherein
R₁ is selected from:
(viii) amino, alkyl amino, aryl amino, heteroaryl amino;
(ix) alkylthio, sulfinyl, sulfonyl, sulfamoyl;
(xx) alkyloxy, Alkanoyl, alkoxycarbonyl;
(xxi) hydrogen, C₁-C₆ alkyl, cycloalkyl, C₂-C₆ alkenyl, C₂-C₆ alkynyl;
(xxii) aryl, heterocyclic, heteroaryl;
(xxiii) C₁-C₆ trifluoroalkyl, cyano and;
(xxiv) groups of the formula (a): 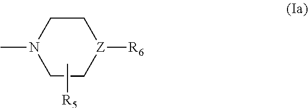
wherein:
R₅ represents hydrogen, C₁-C₄ alkyl, oxo;
Z is CH, when R₆ is hydrogen; or Z—R₆ is O; or Z is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁C₆alkyl)aminocarbonyl, each of which is substituted with from 0 to 4 substituents independently chosen from halogen, hydroxy, cyano, amino, —COOH and oxo;
ring A is aryl, heterocyclic, heteroaryl;
R₂ is hydroxyl, halogen, amino, nitro, cyano, alkyl, alkenyl, alkynyl, alkanoyl, alkylthio, sulfonyl, sulfinyl, alkoxy, alkoxycarbonyl, carbamoyl, acylamine, sulfamoyl or sulfonamide;
or R₂ is a aryl, heterocyclic or heteroaryl that is optionally substituted with hydroxyl, halogen, amino, nitro, cyano, alkyl, acyl, sulfonyl, sulfinyl, alkoxy, carbamoyl, acylamine, sulfamoyl and sulfonamide;
R₃ and R₄ are independently selected from hydrogen or an optionally substituted C₁₋₄ alkyl group;
m is 0-4;
Y is absent or CR₄R₇; and
R₄, R₇ are as defined herein.

4. A process for making compound of claim 1 or its pharmaceutically acceptable salts, hydrates, solvates, crystal forms salts and individual diastereomers thereof.

5. A pharmaceutical composition comprising at least one compound of claim 1 or its pharmaceutically acceptable salts, hydrates, solvates, crystal forms salts and individual diastereomers thereof, and a pharmaceutically acceptable carrier.

6. A compound selected from the group consisting of:

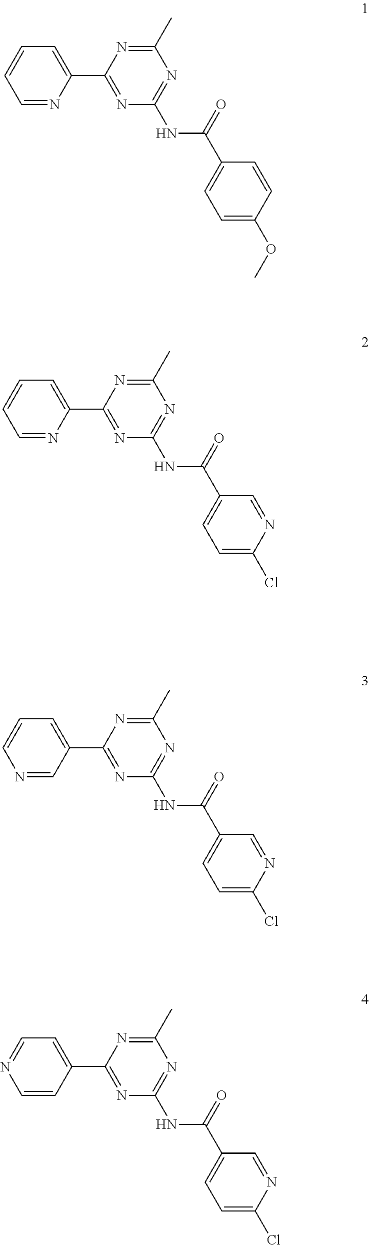

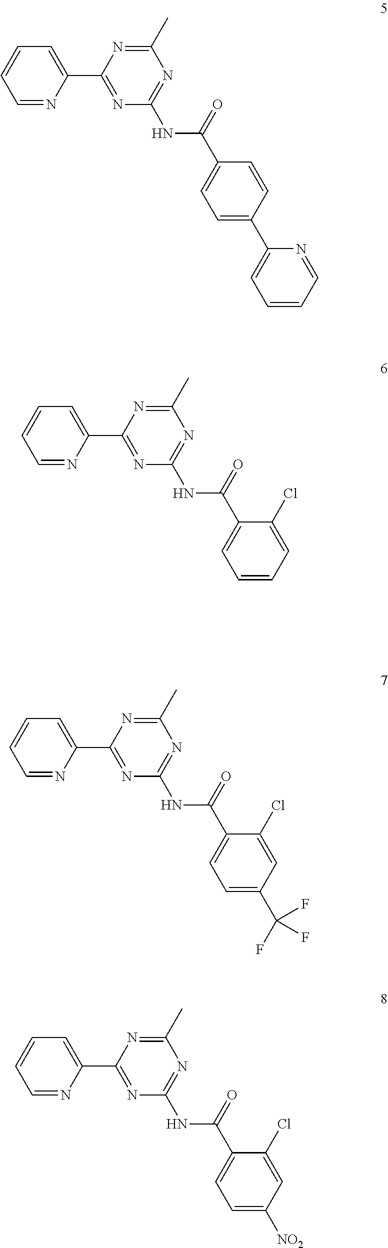

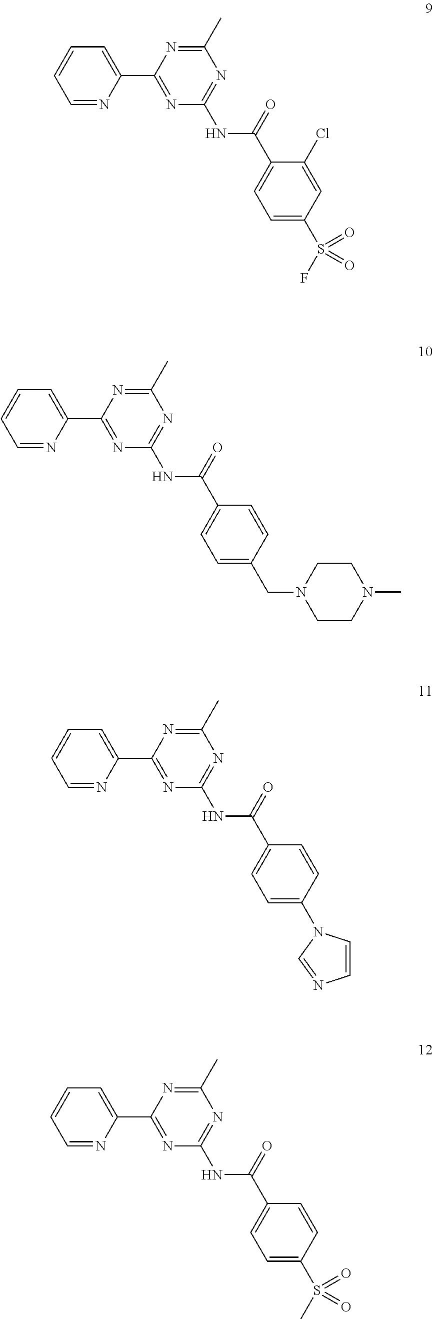

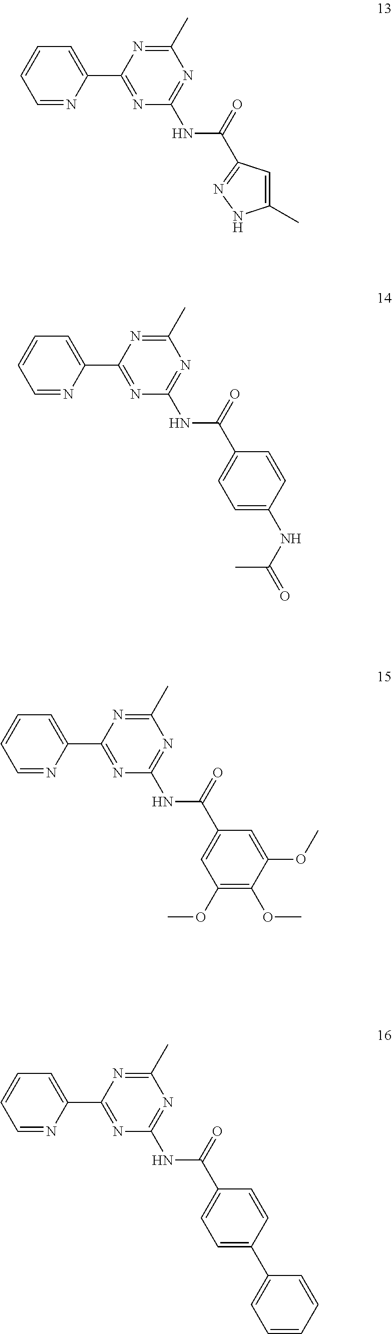

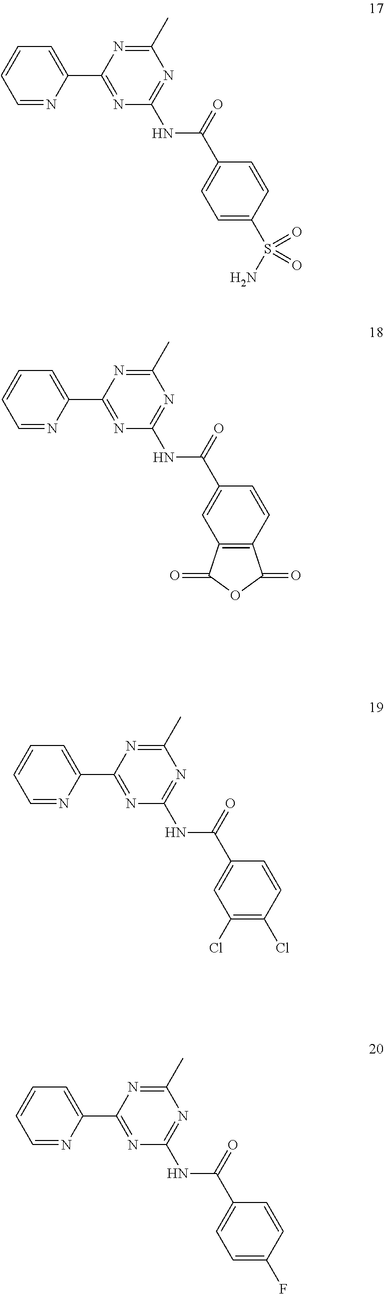

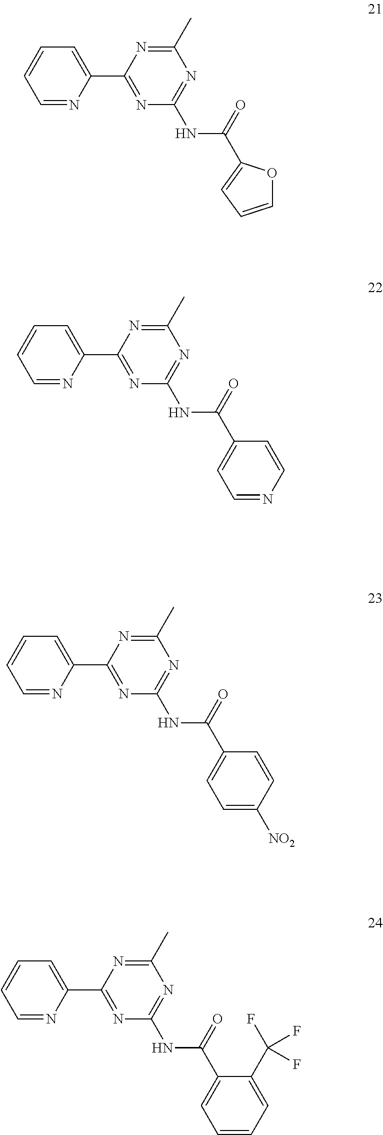

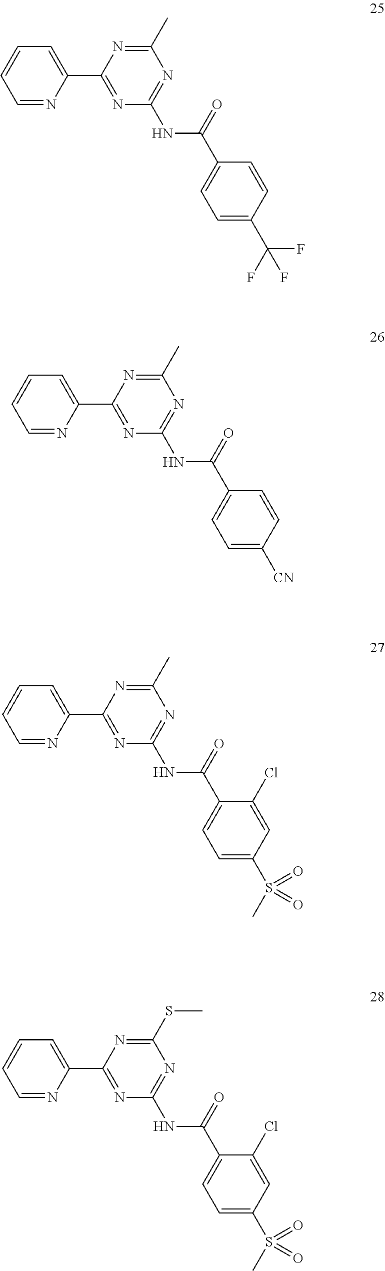

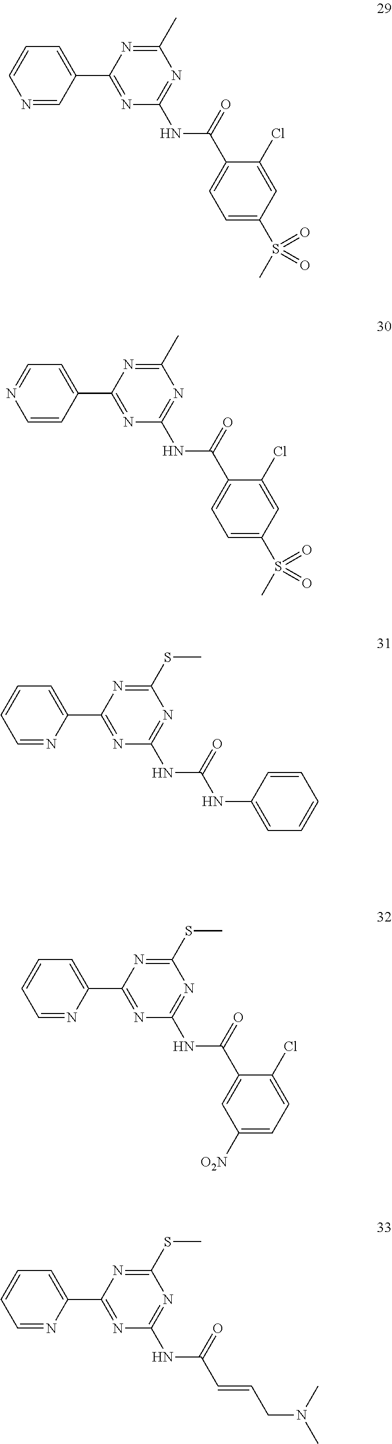

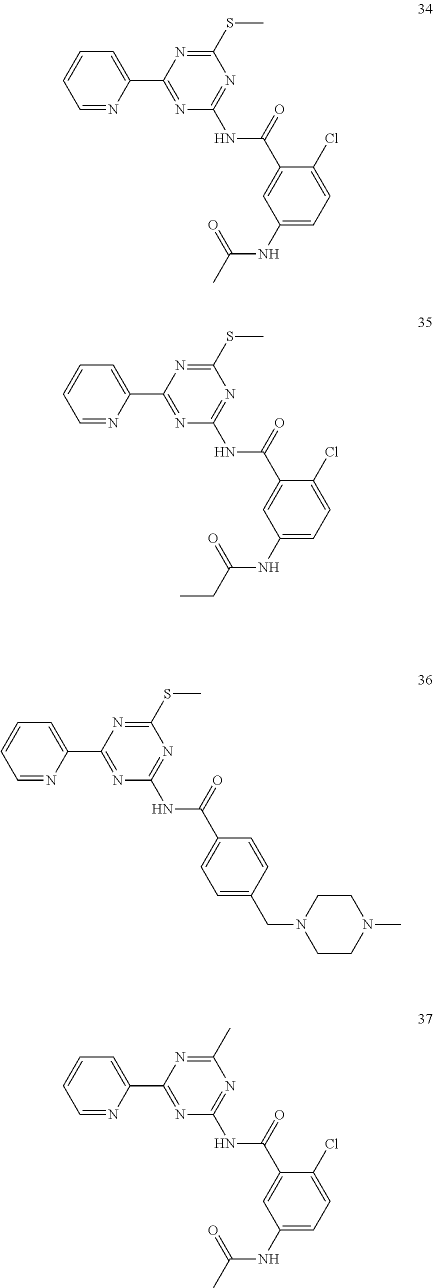

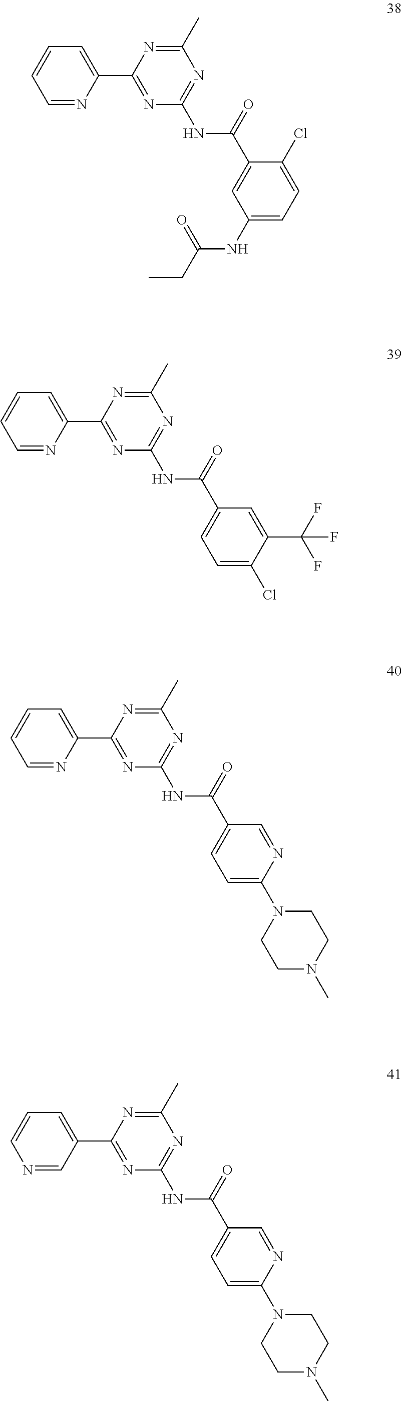

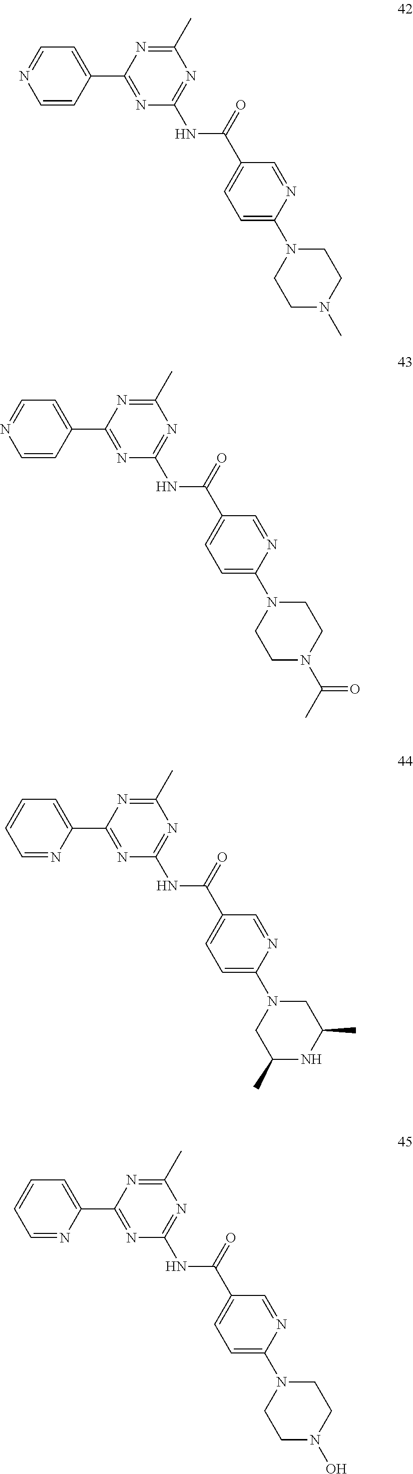

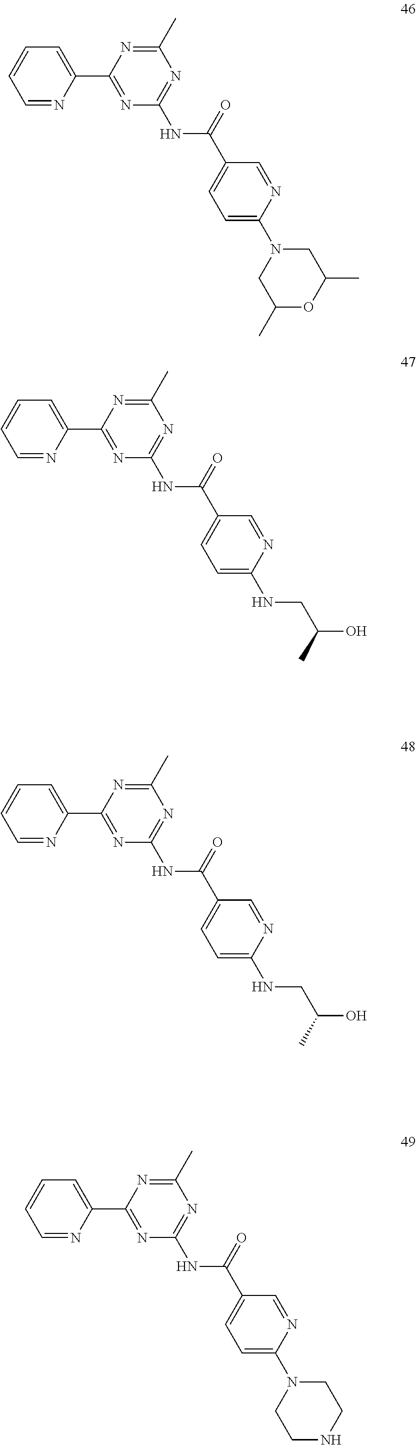

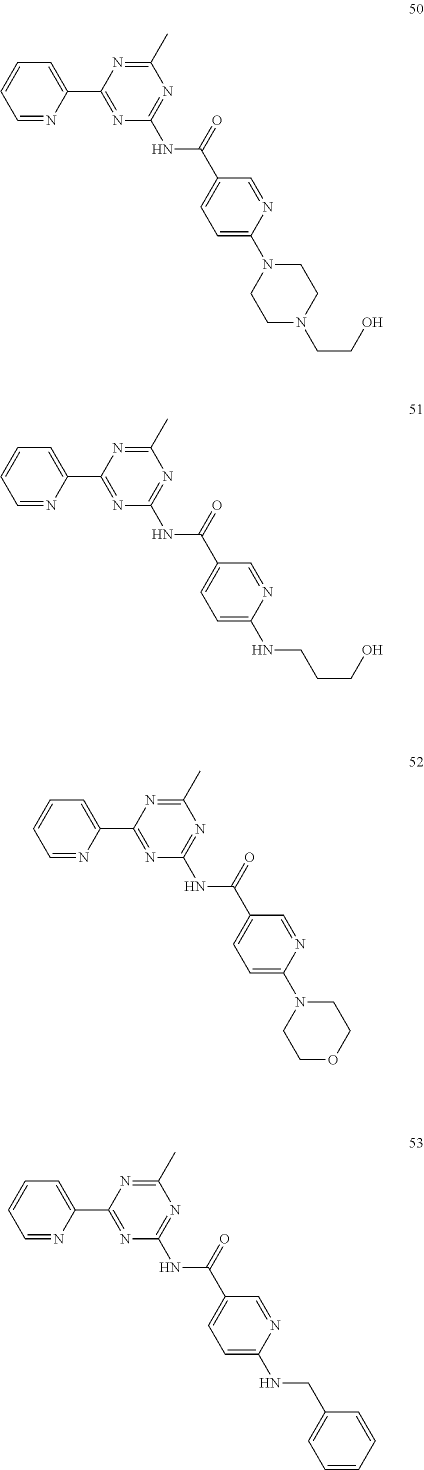

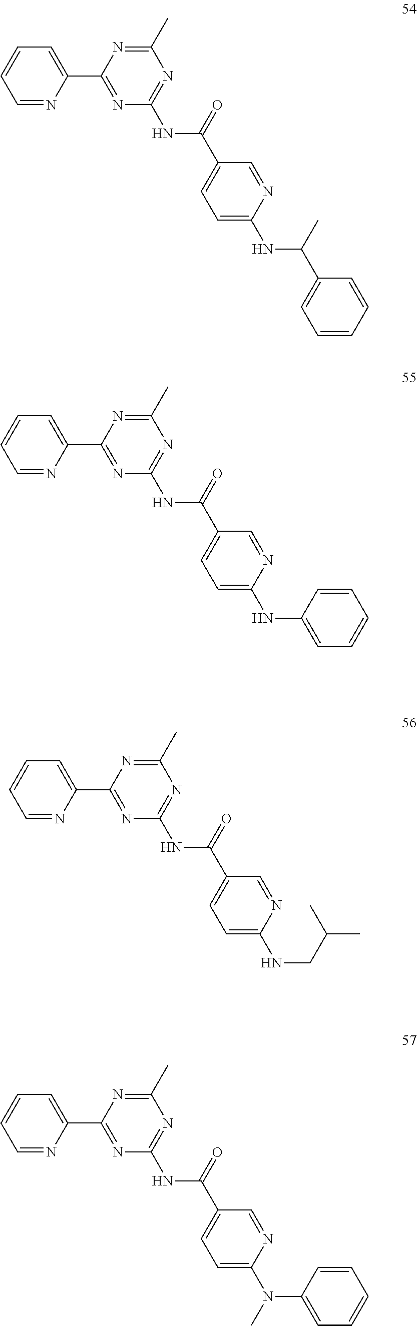

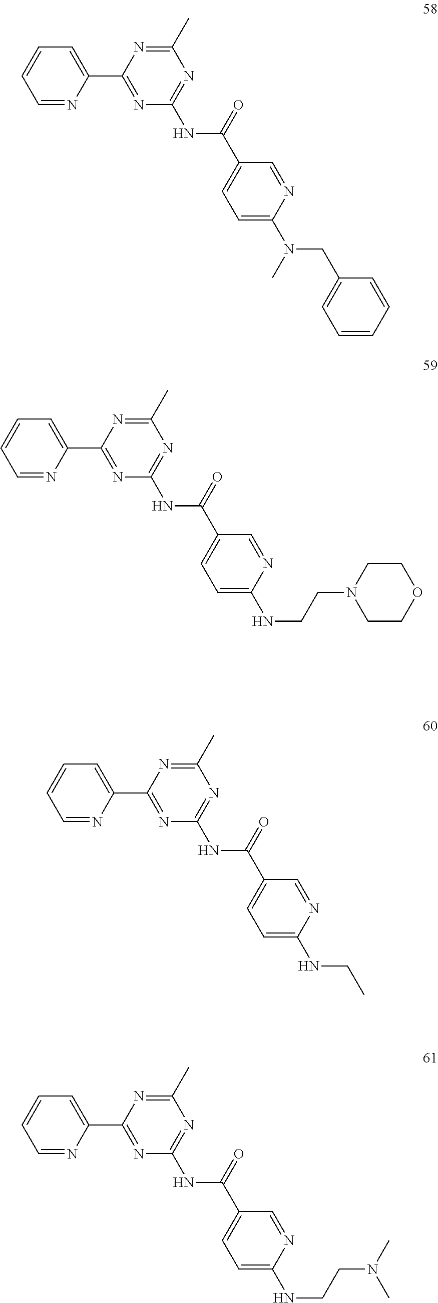

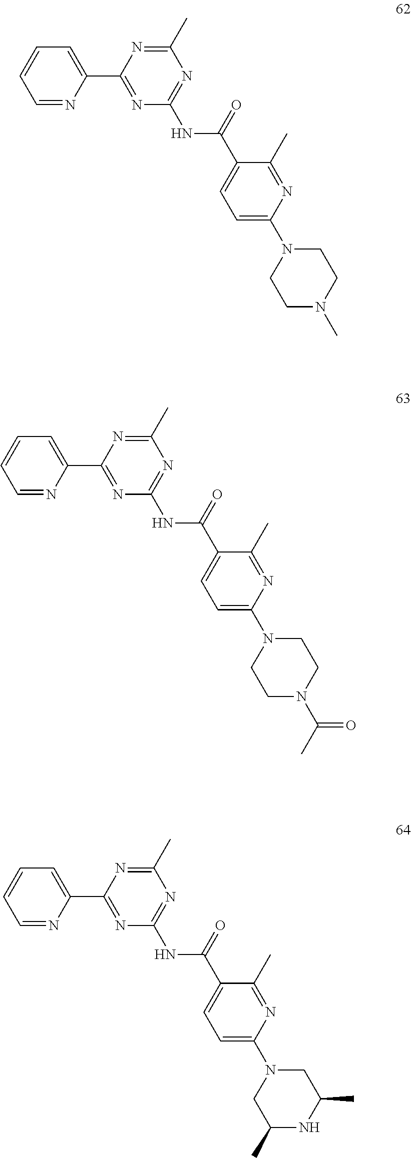

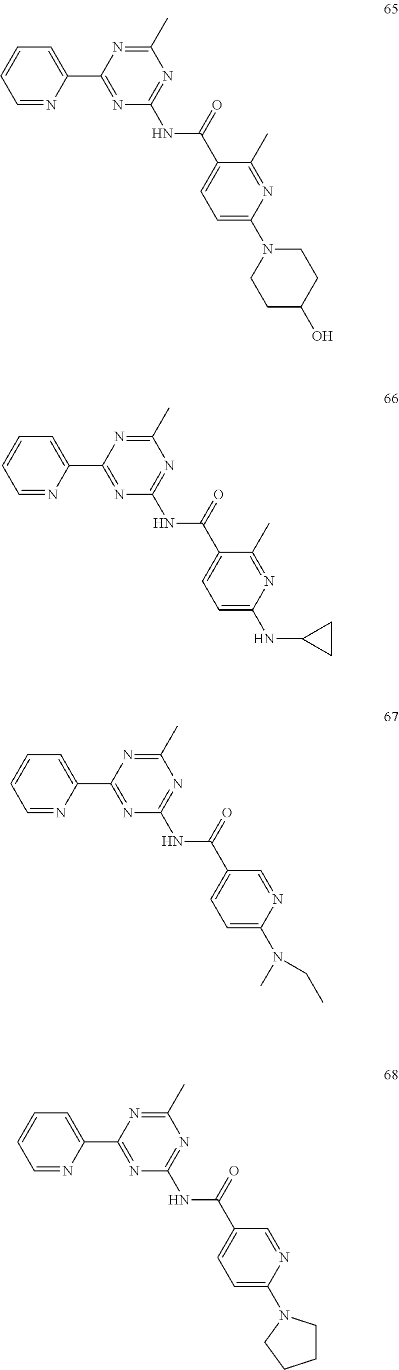

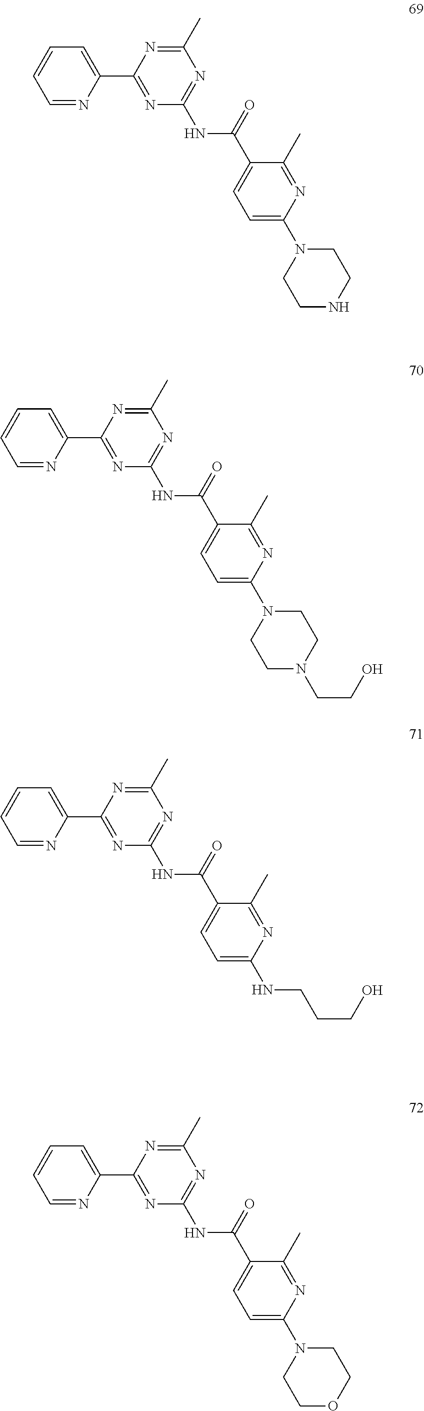

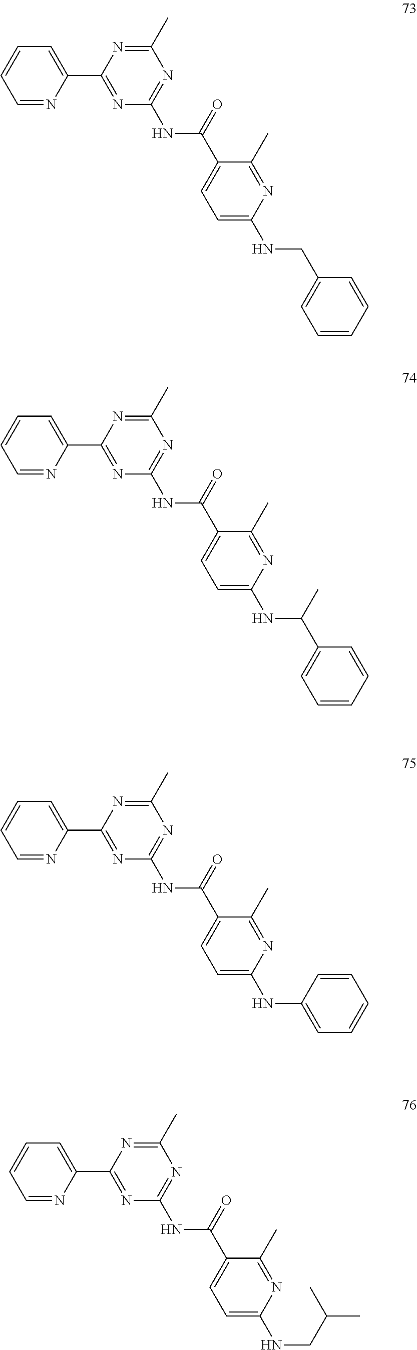

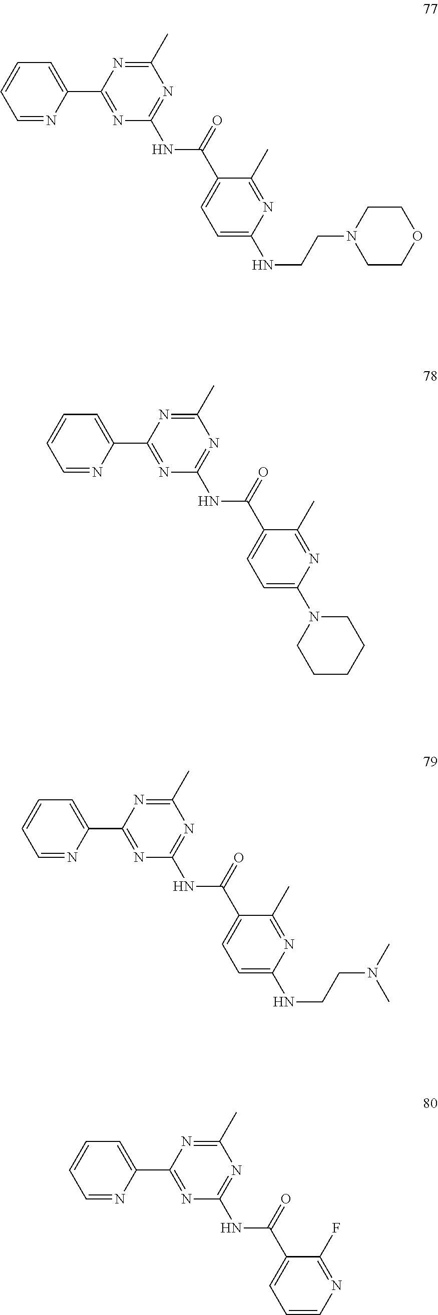

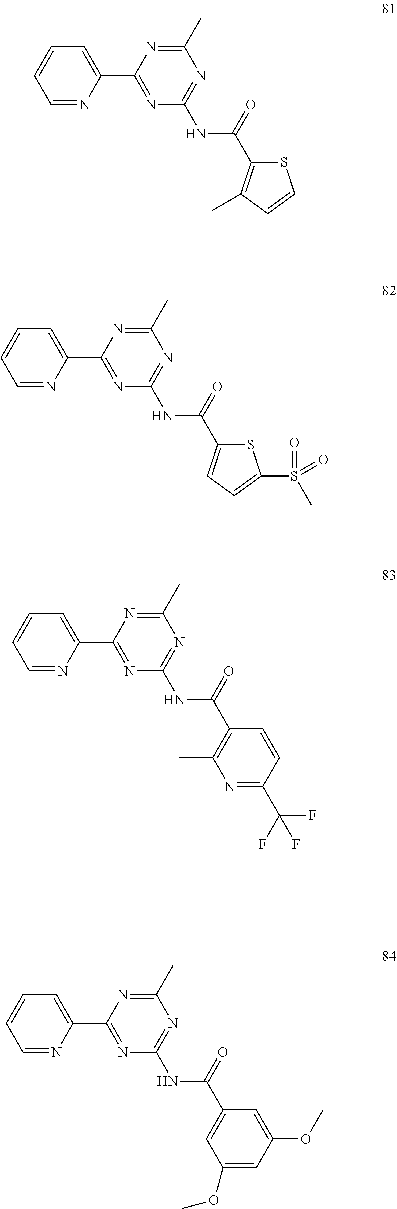

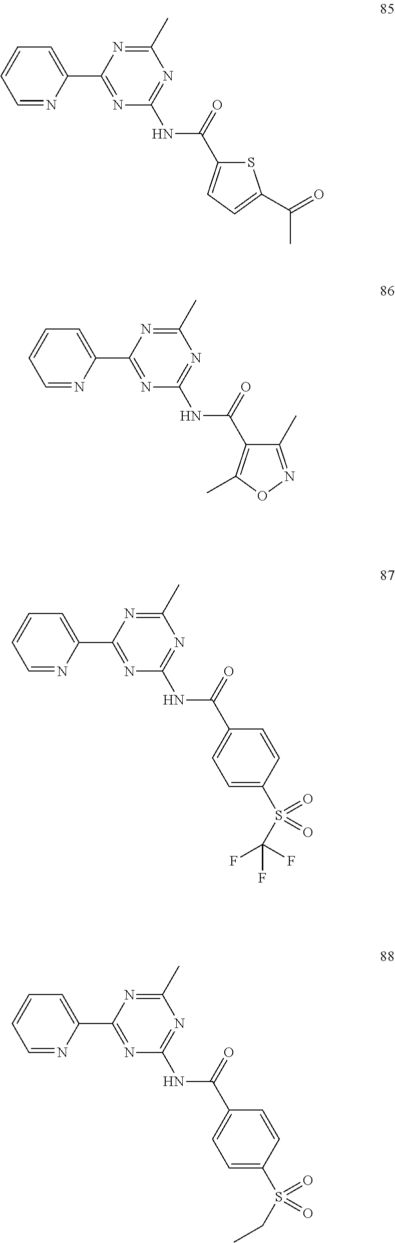

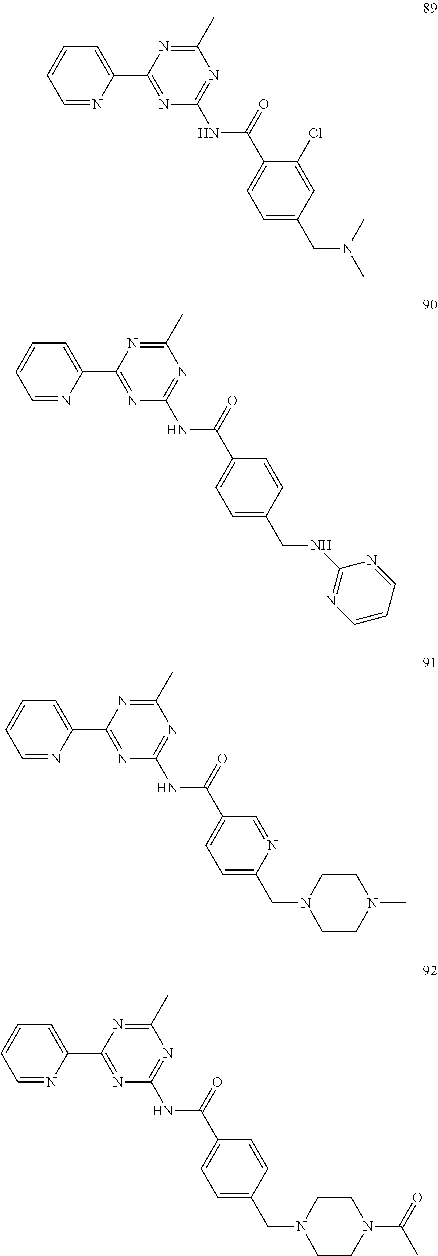

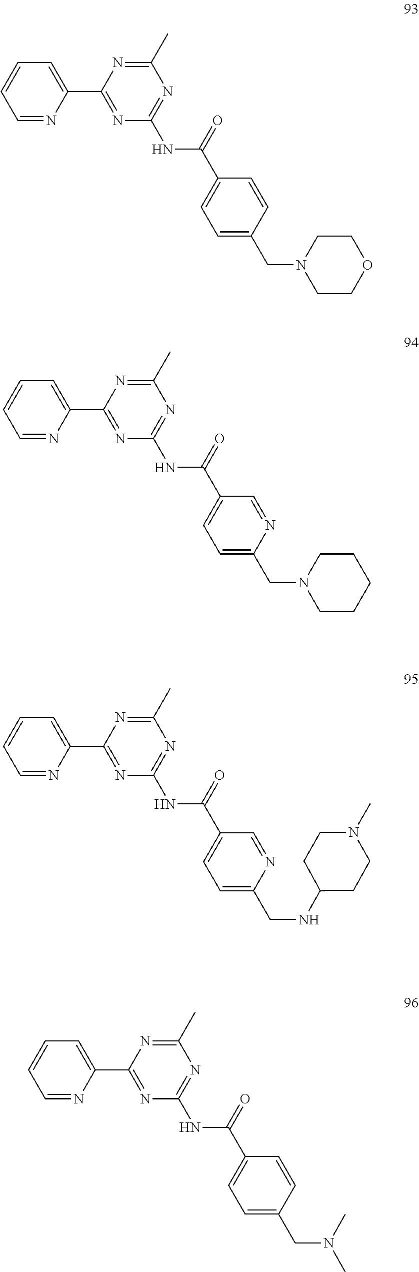

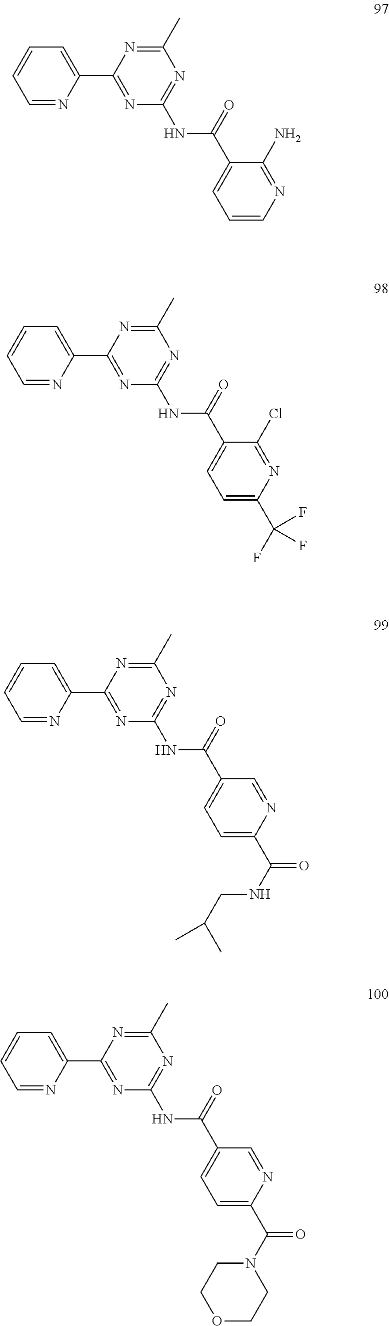

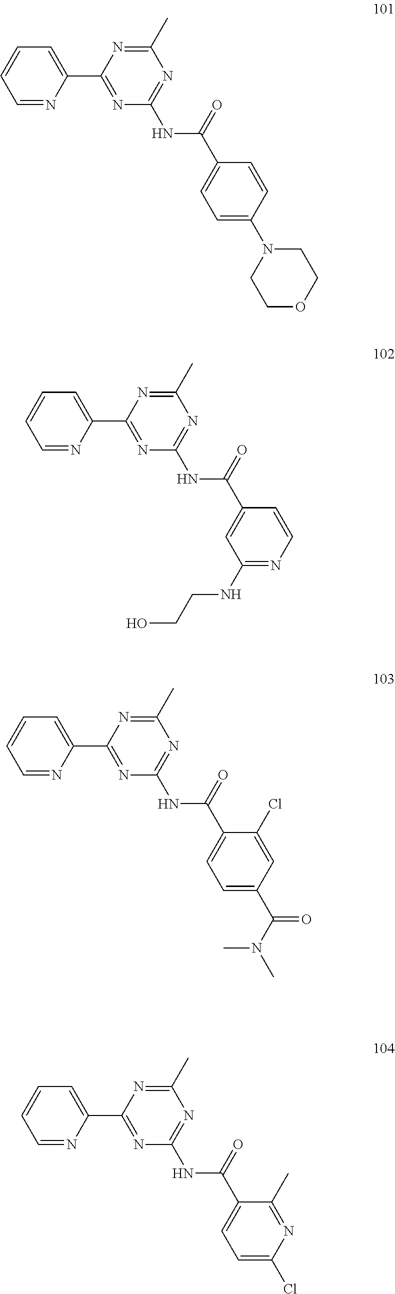

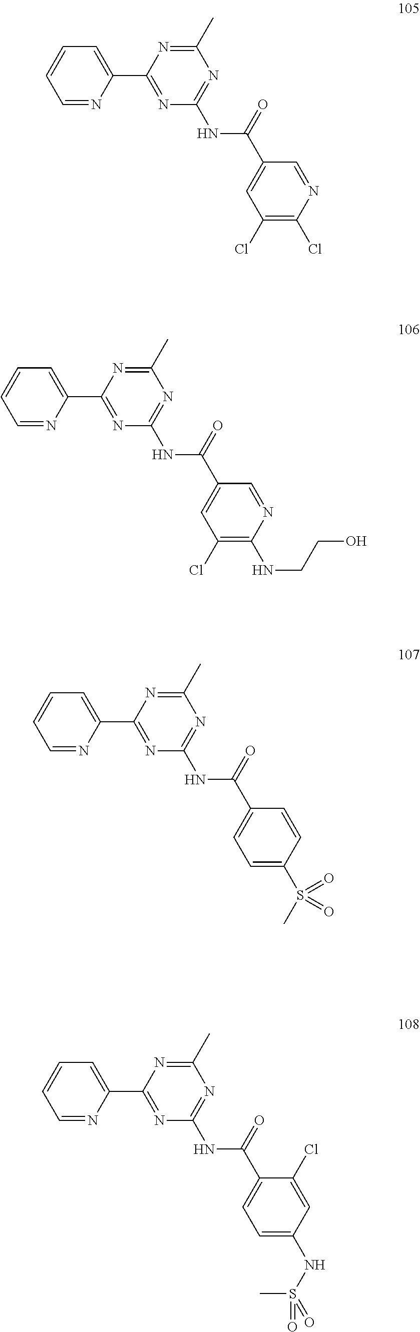

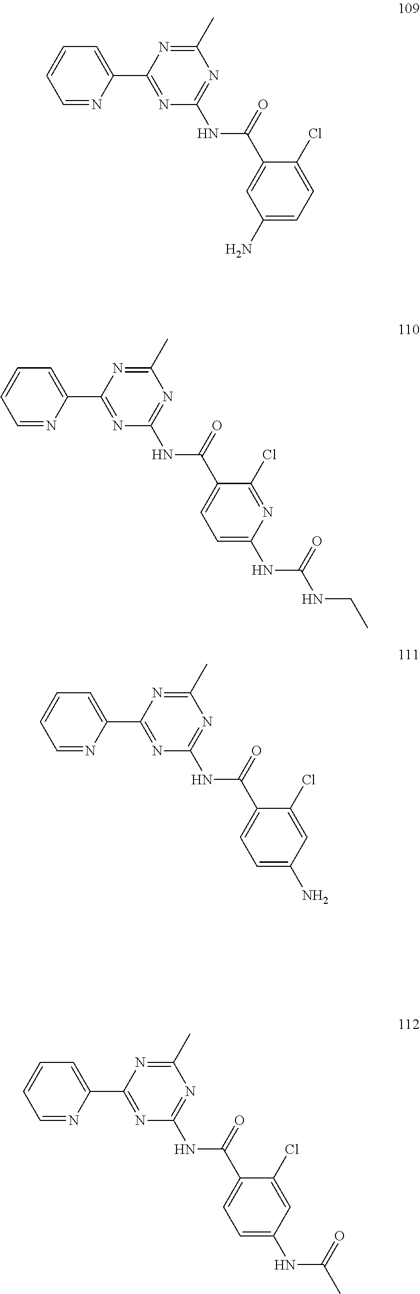

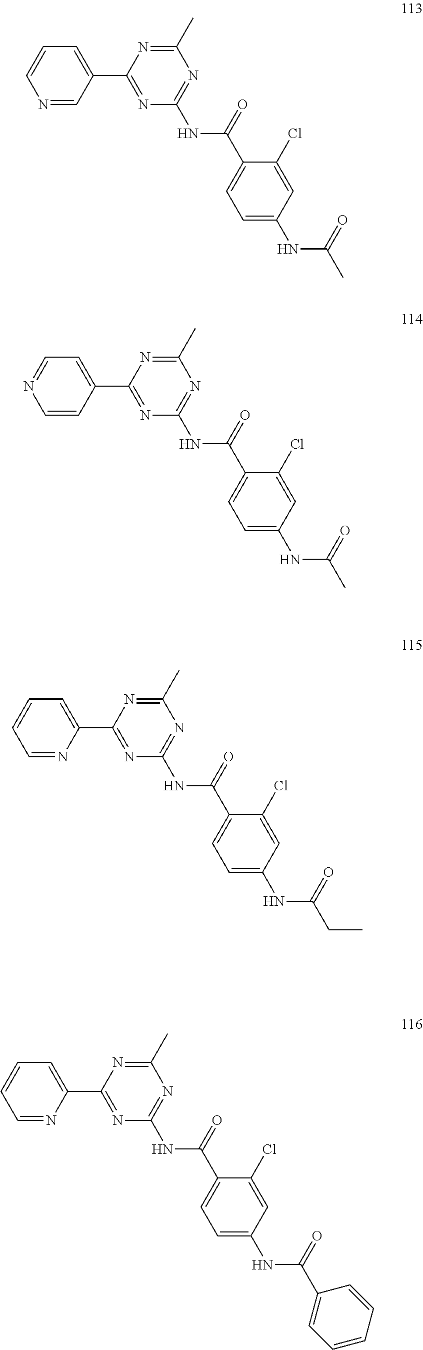

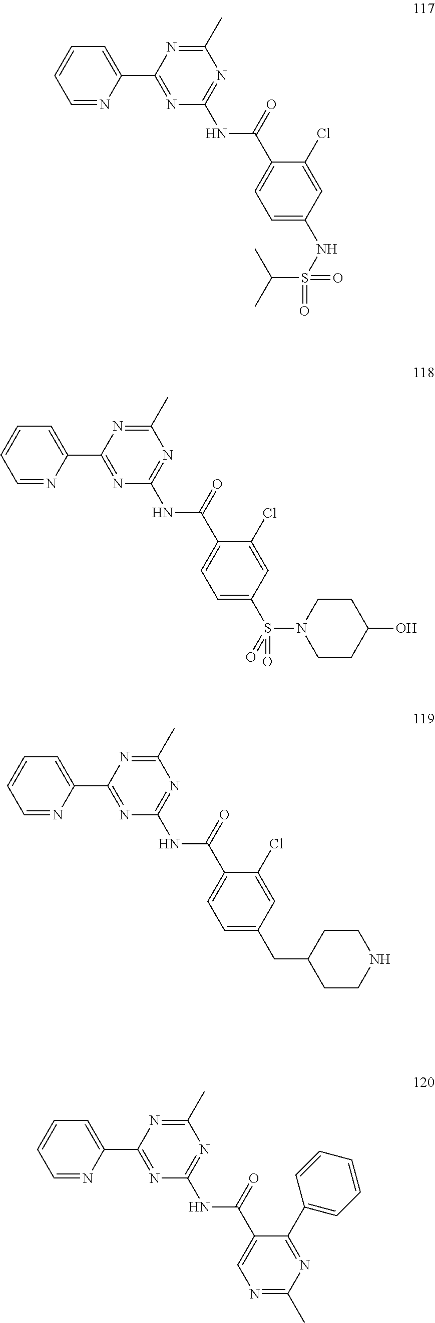

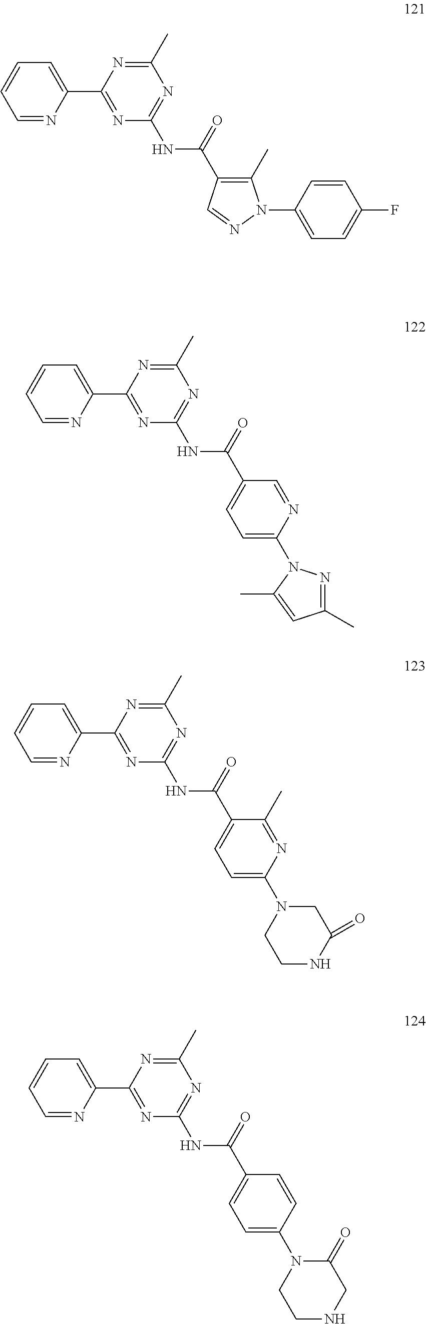

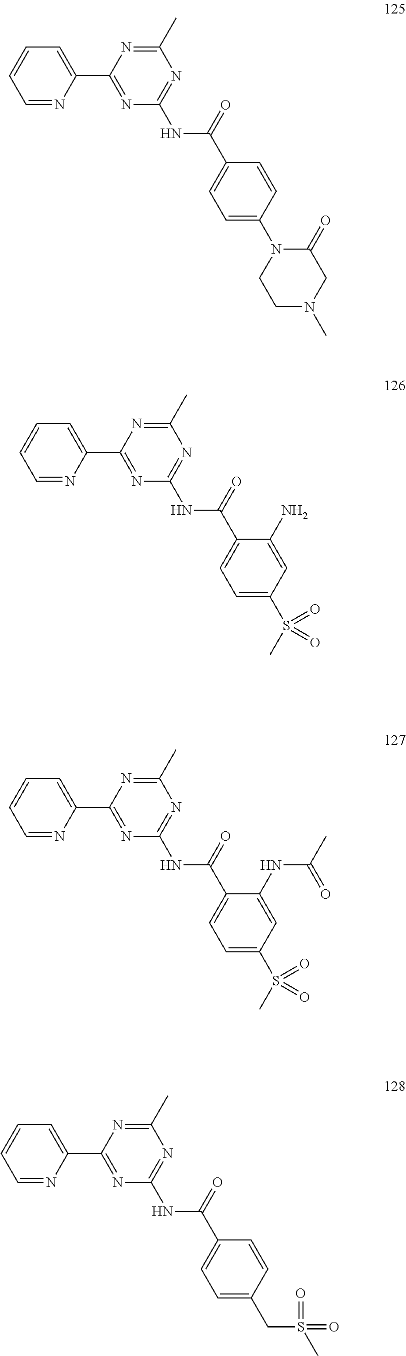

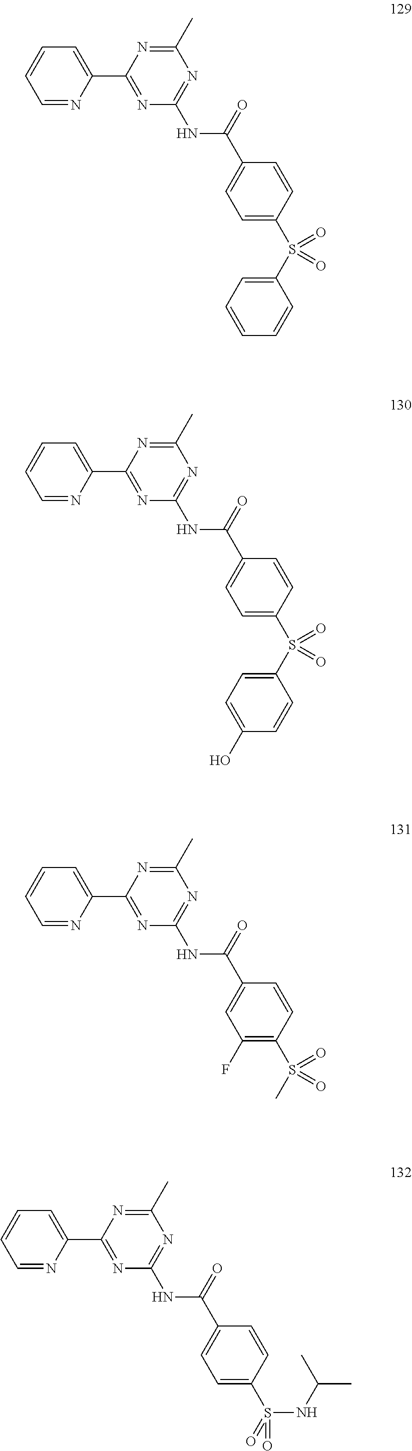

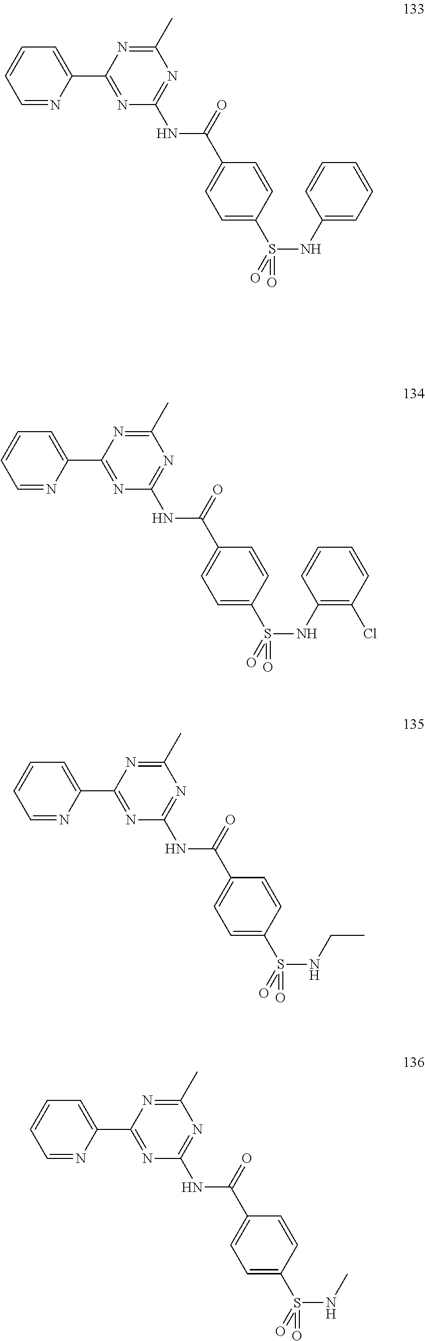

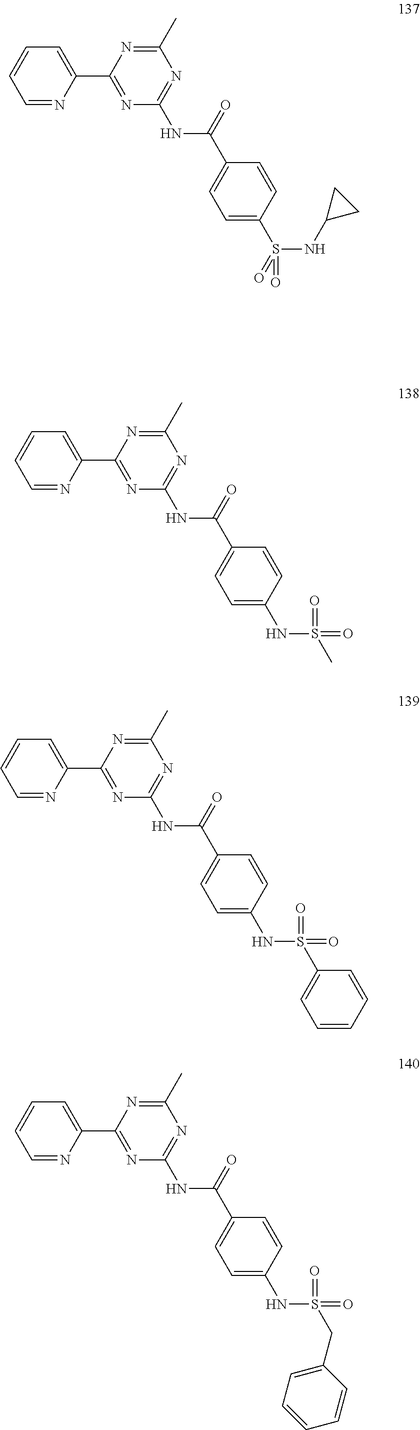

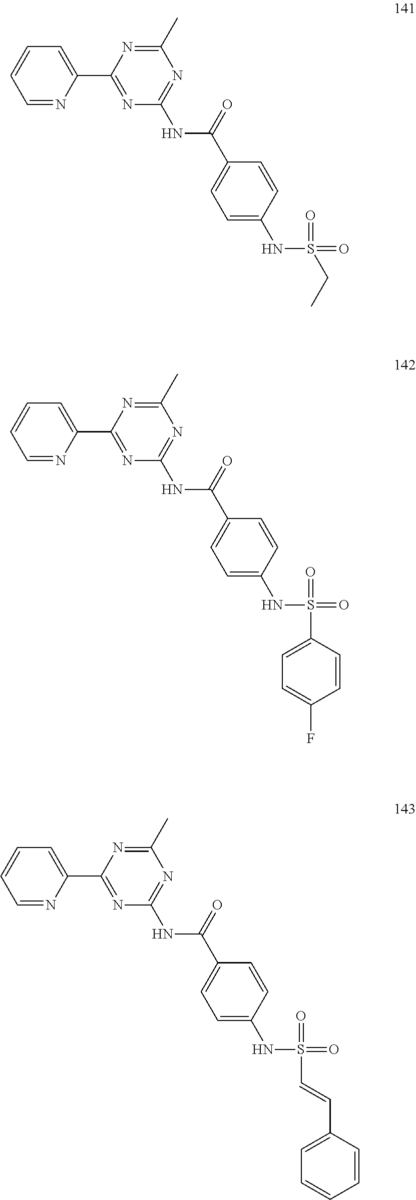

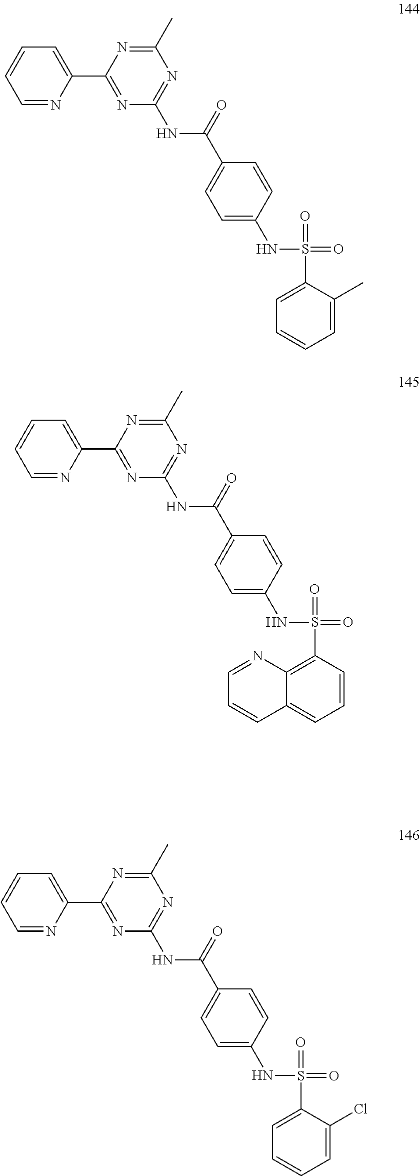

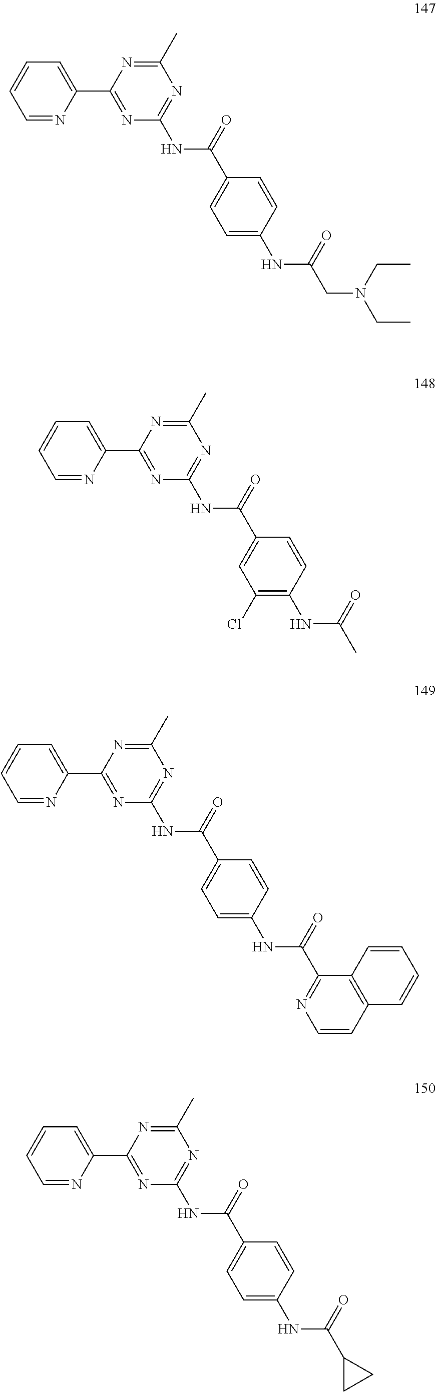

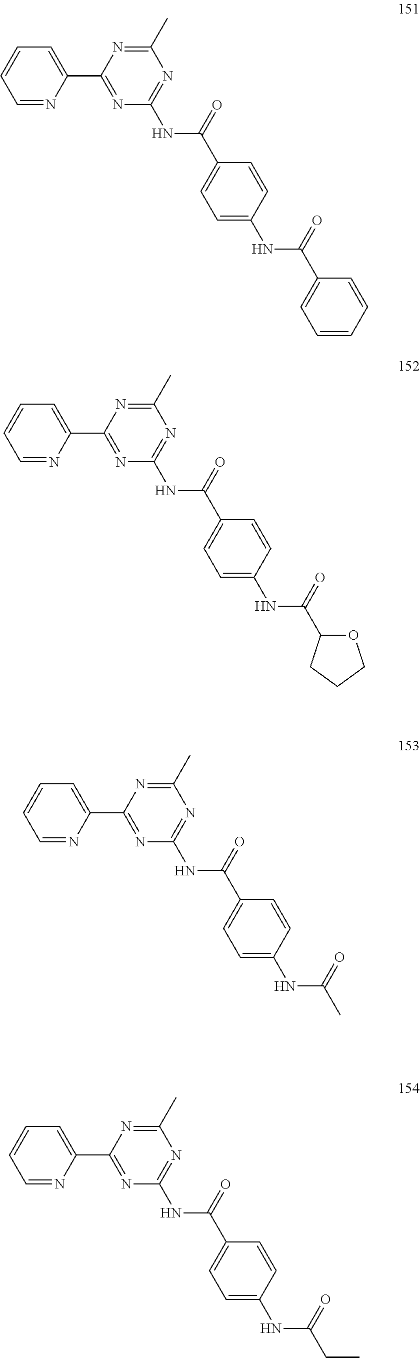

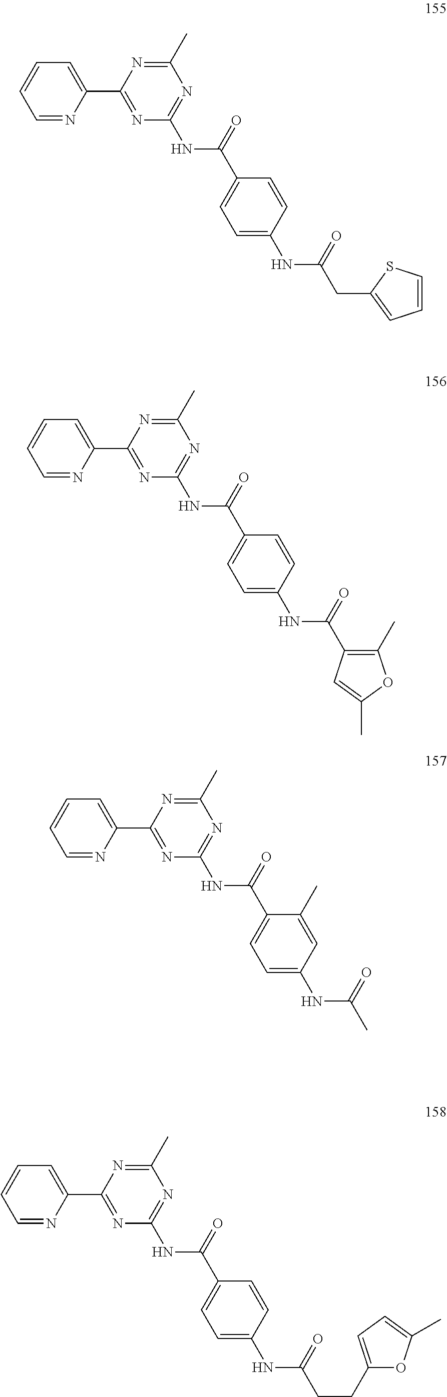

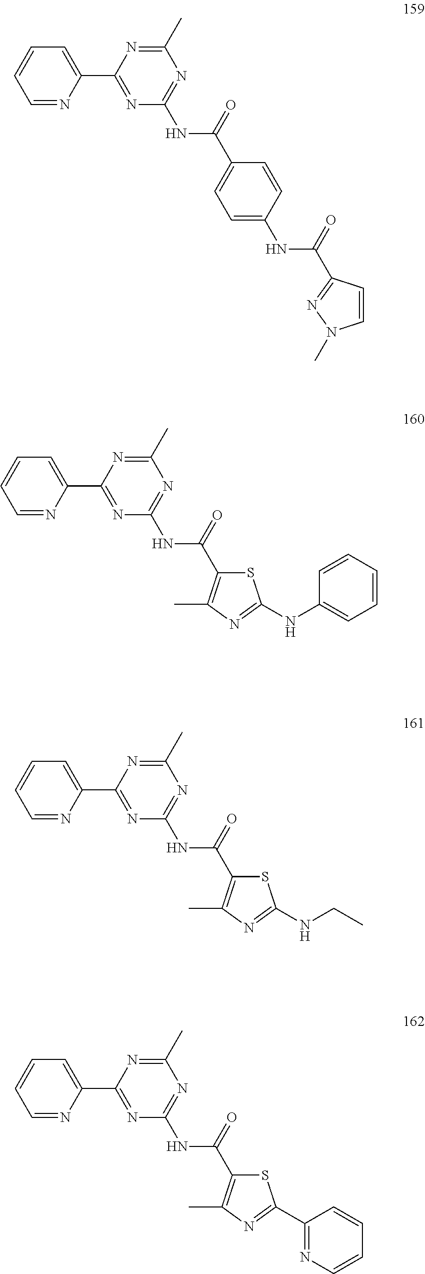

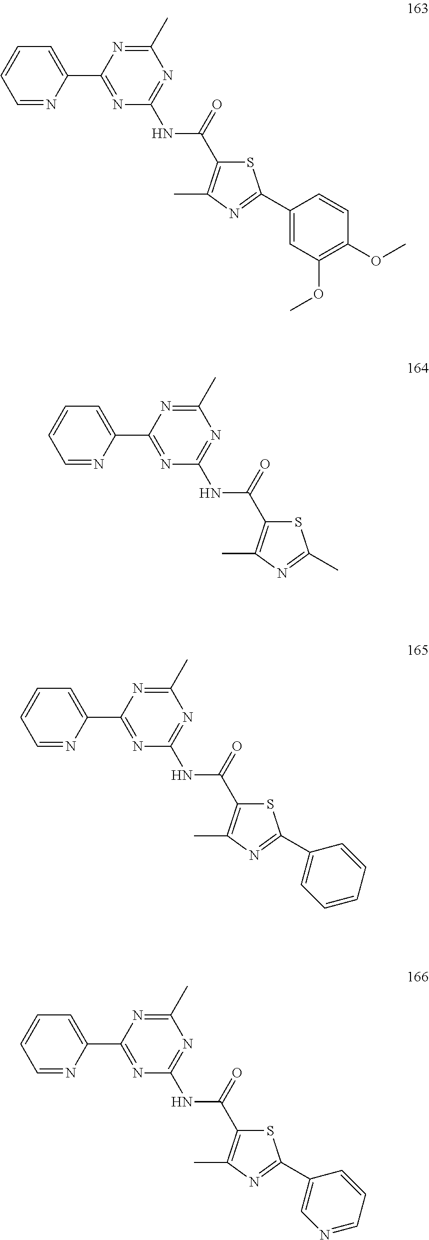

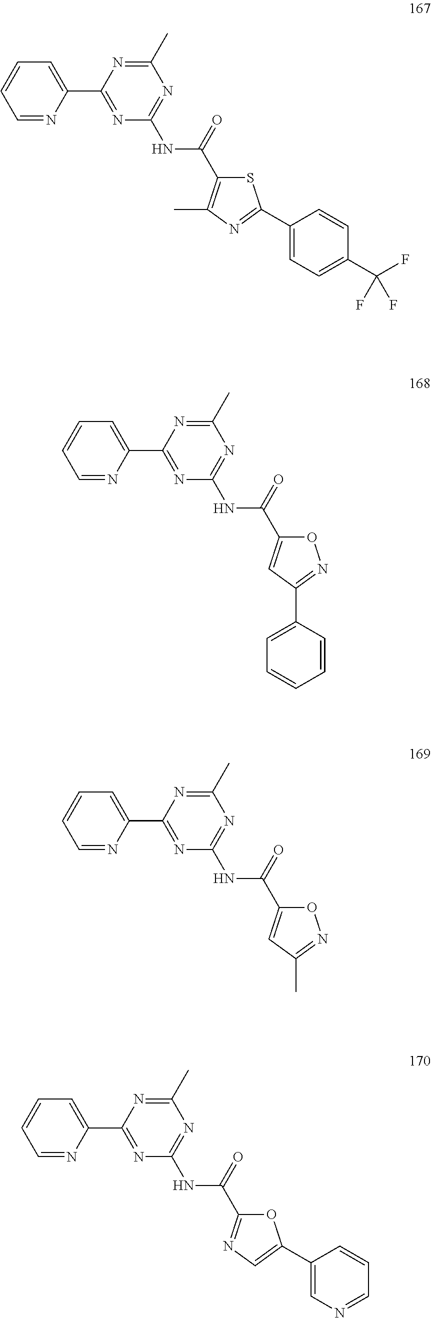

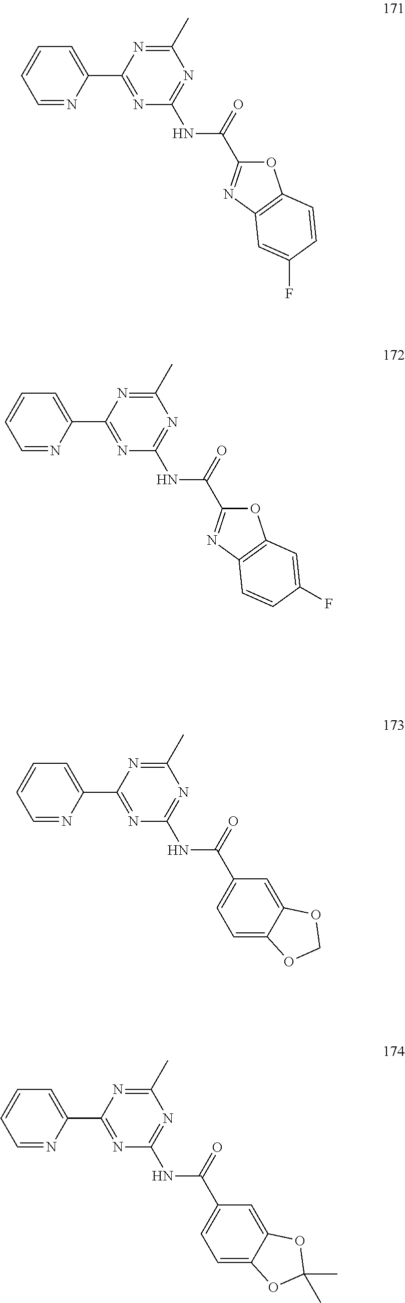

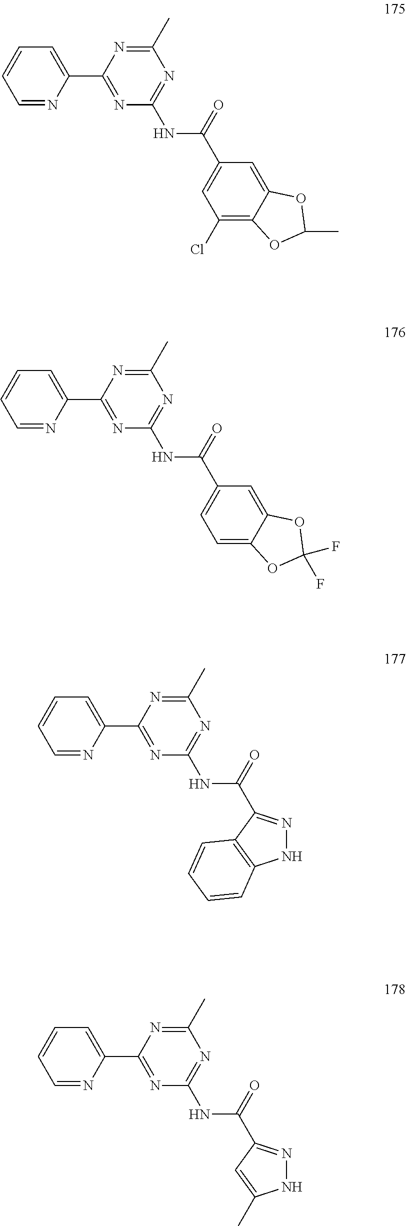

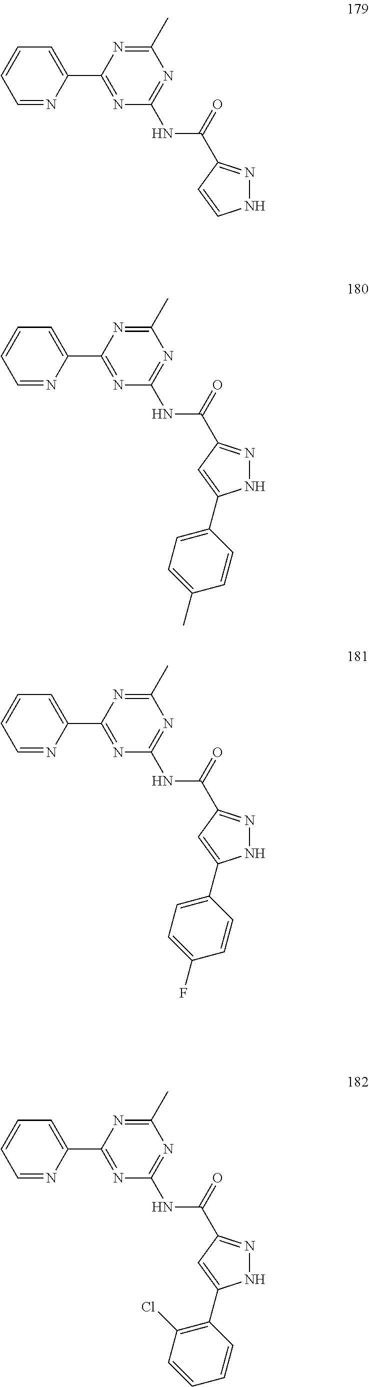

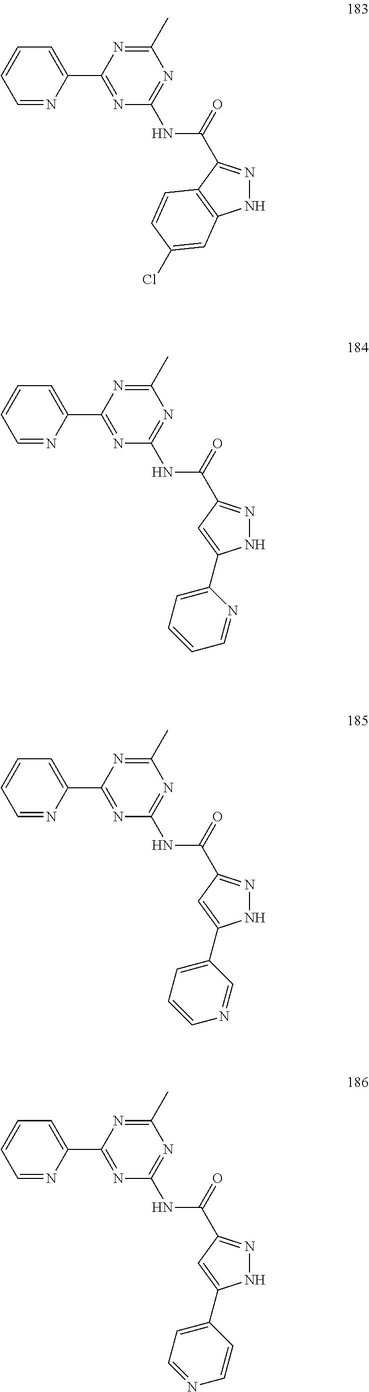

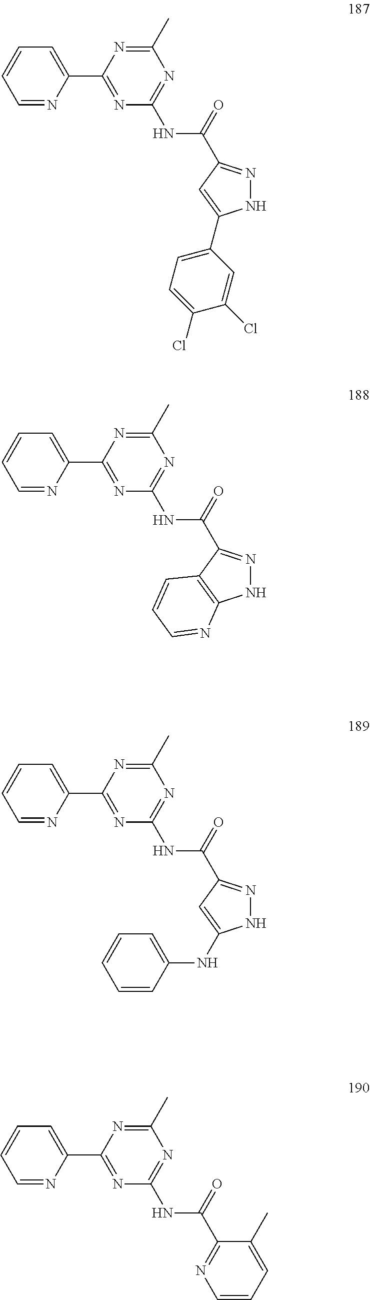

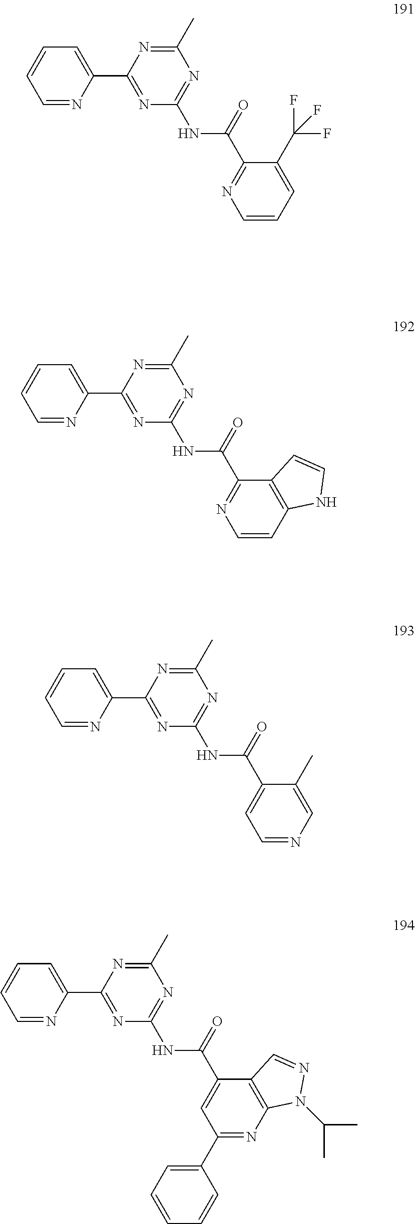

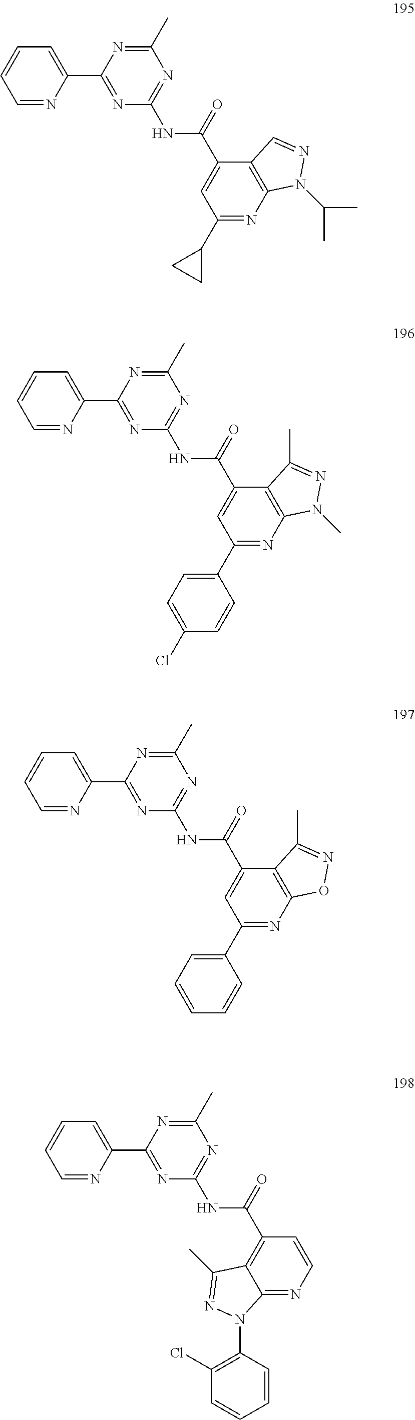

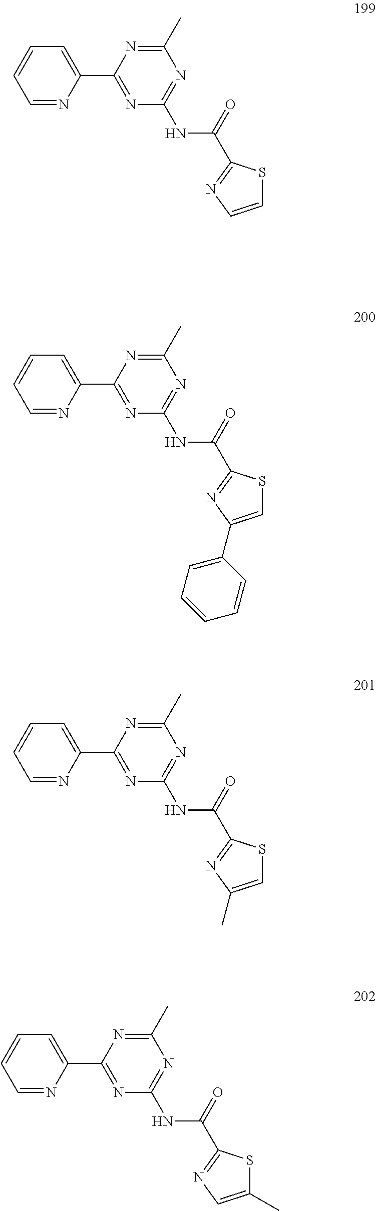

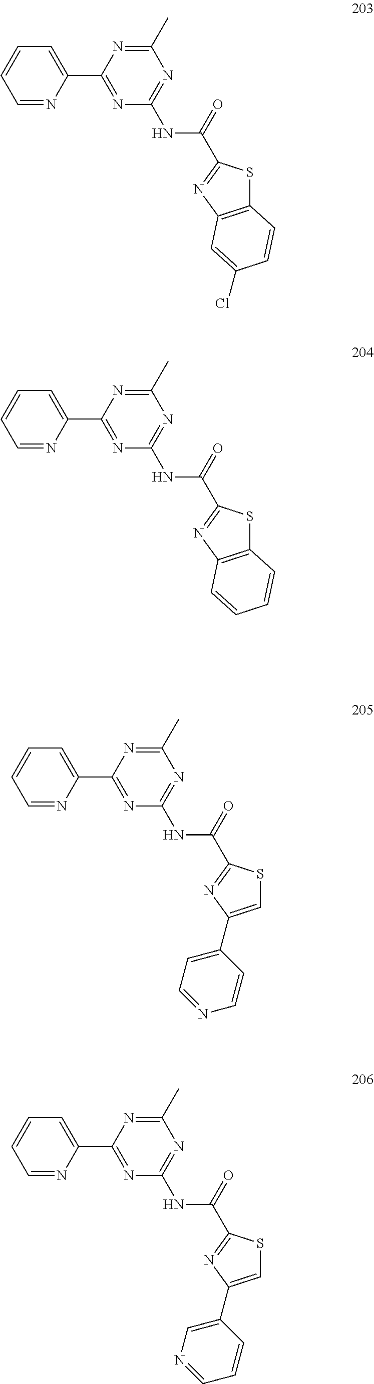

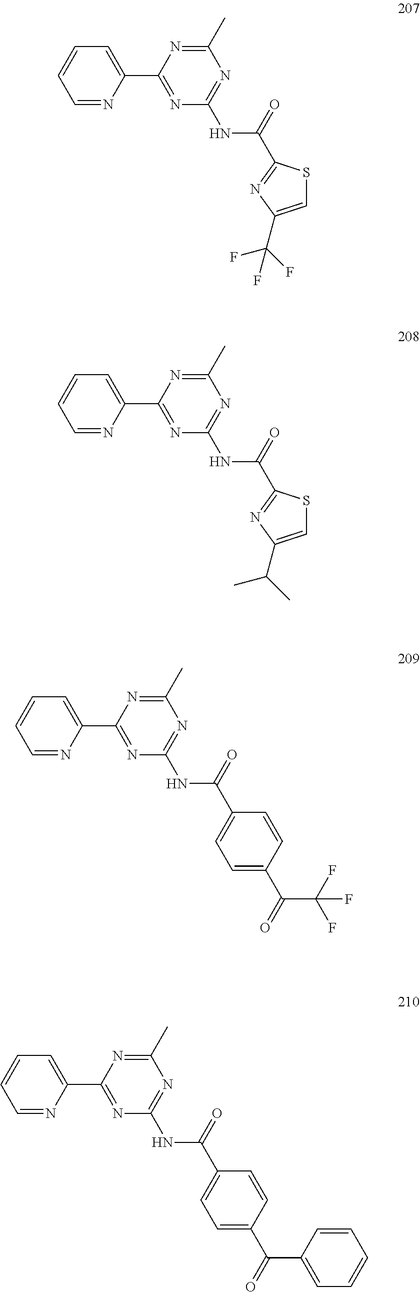

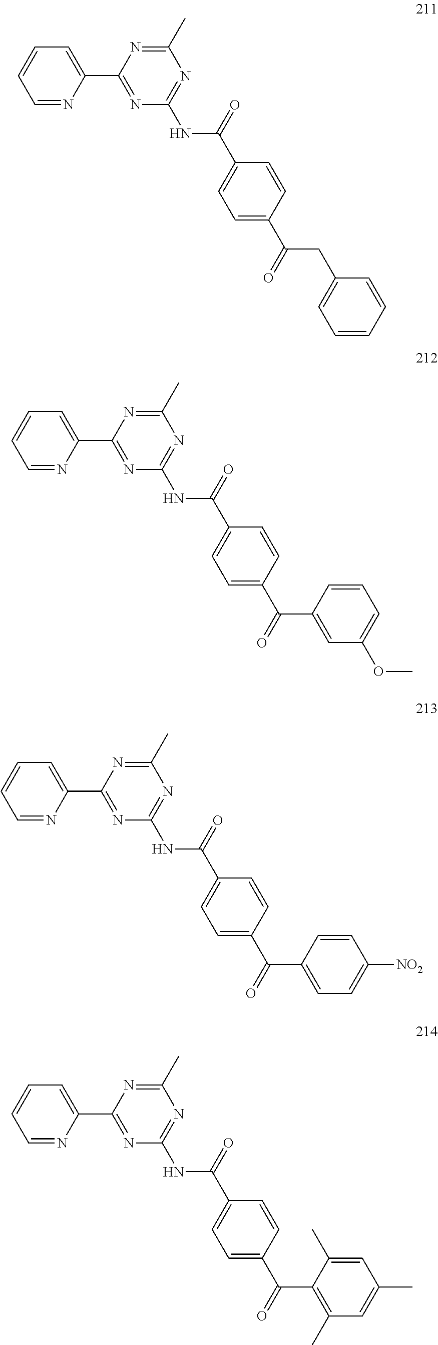

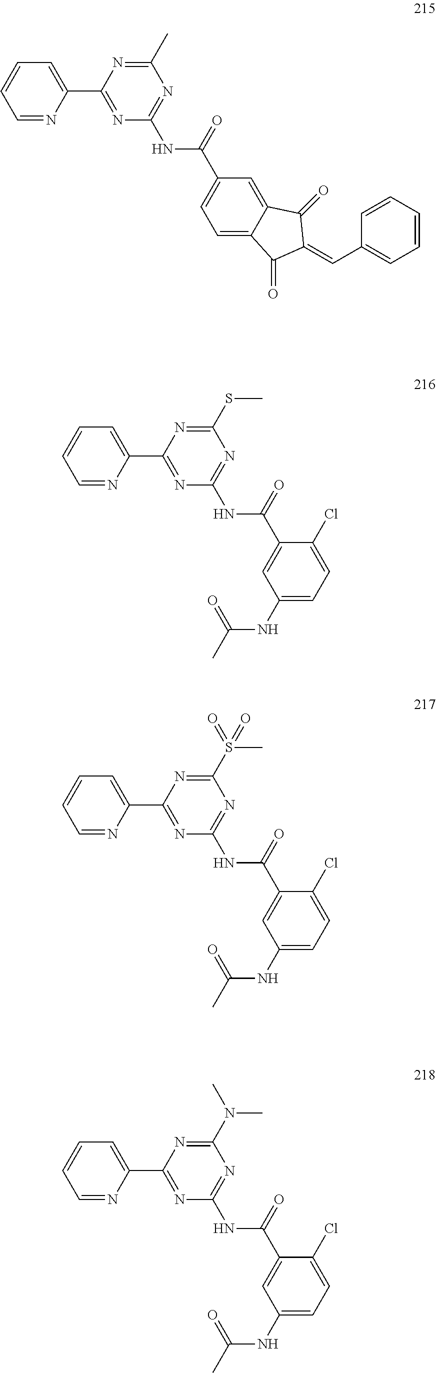

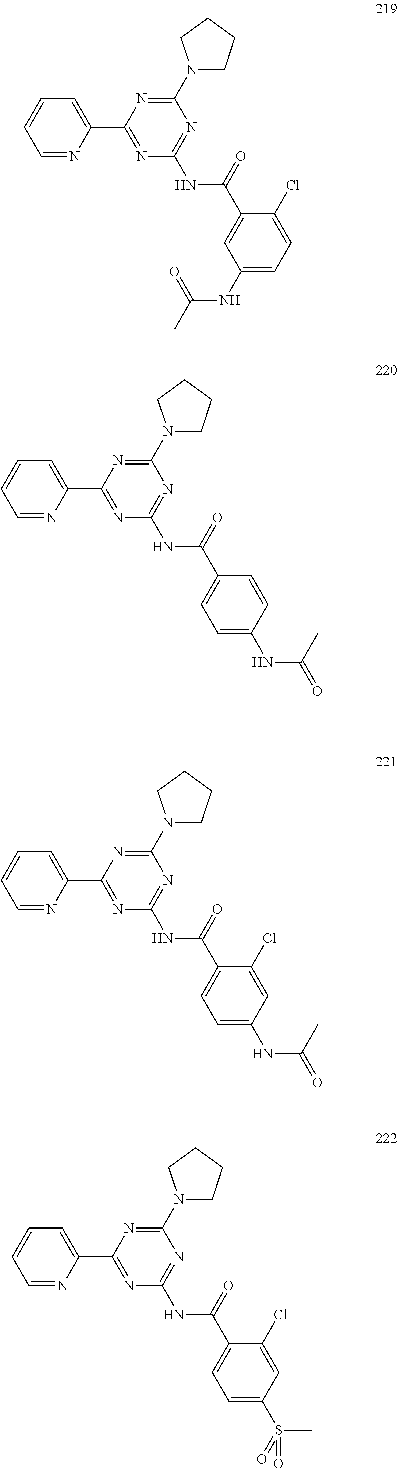

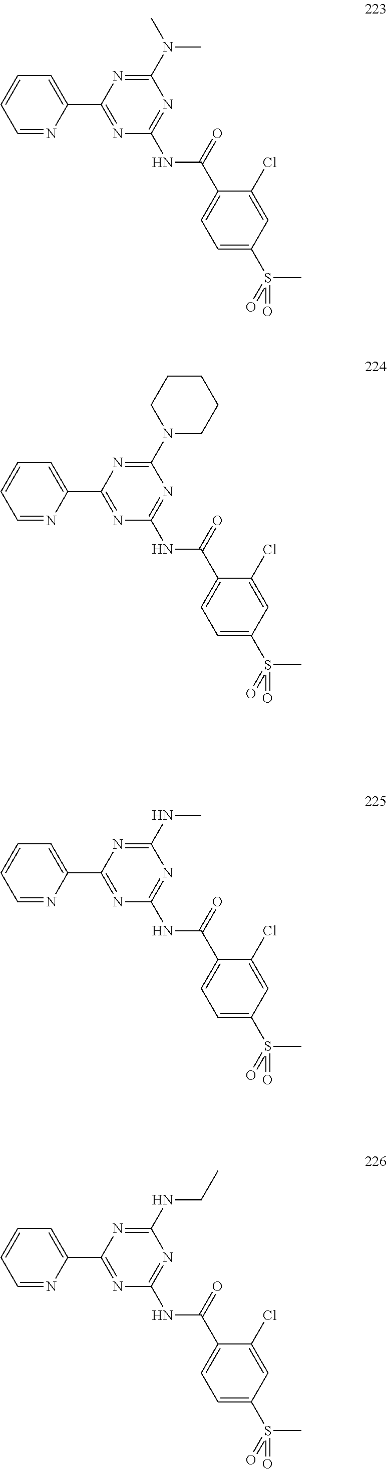

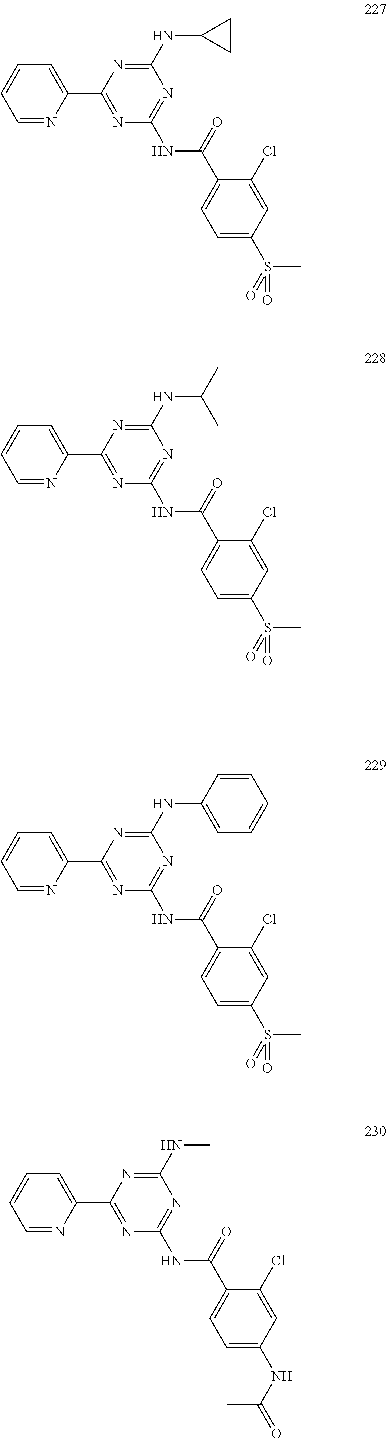

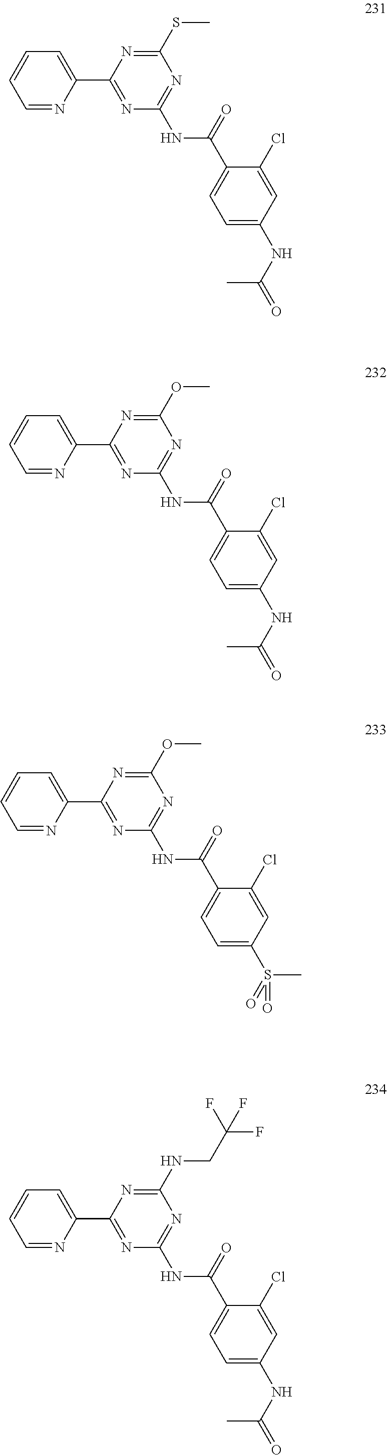

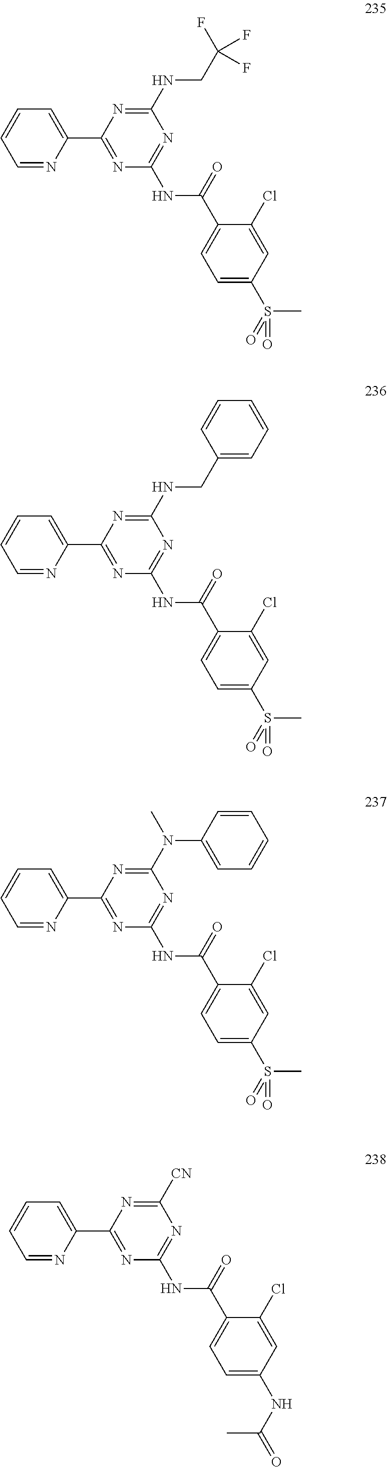

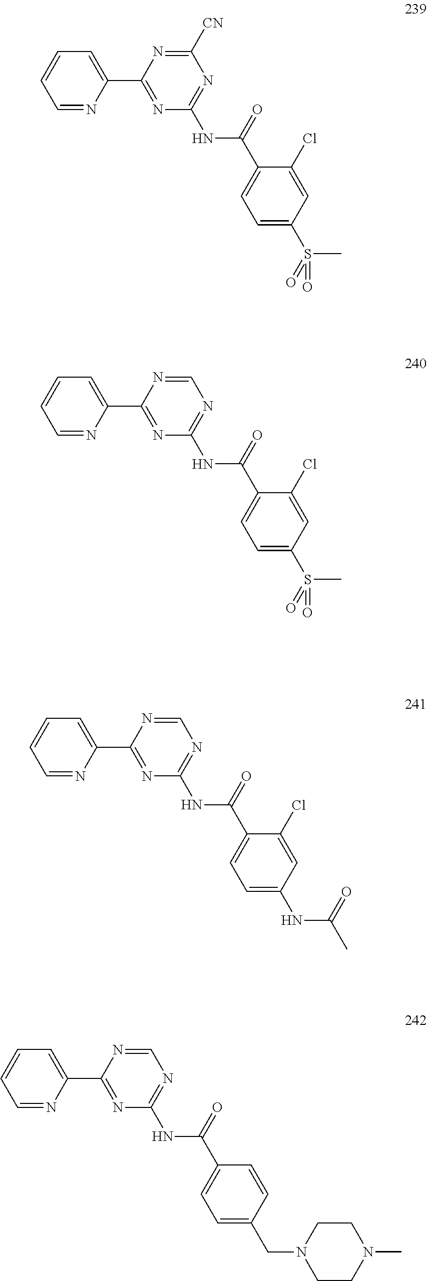

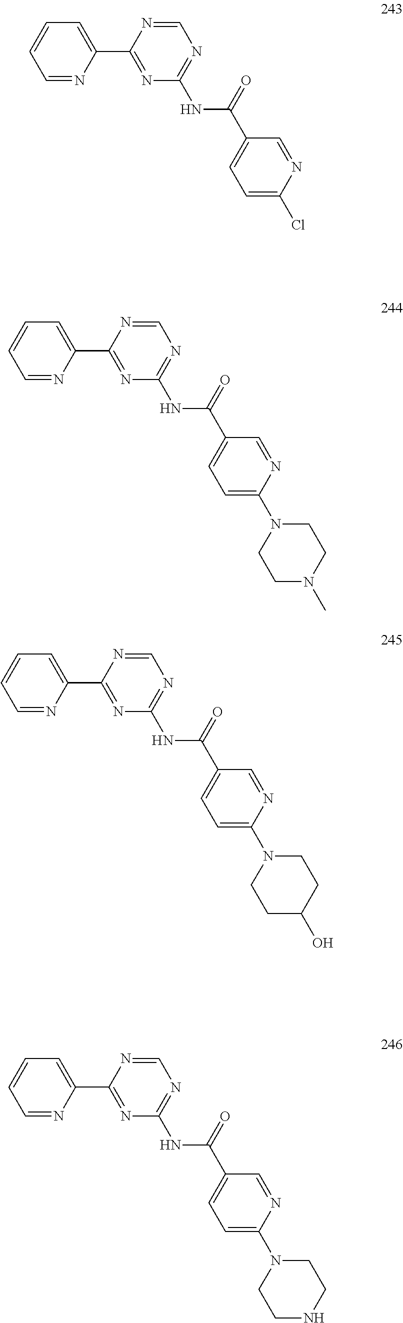

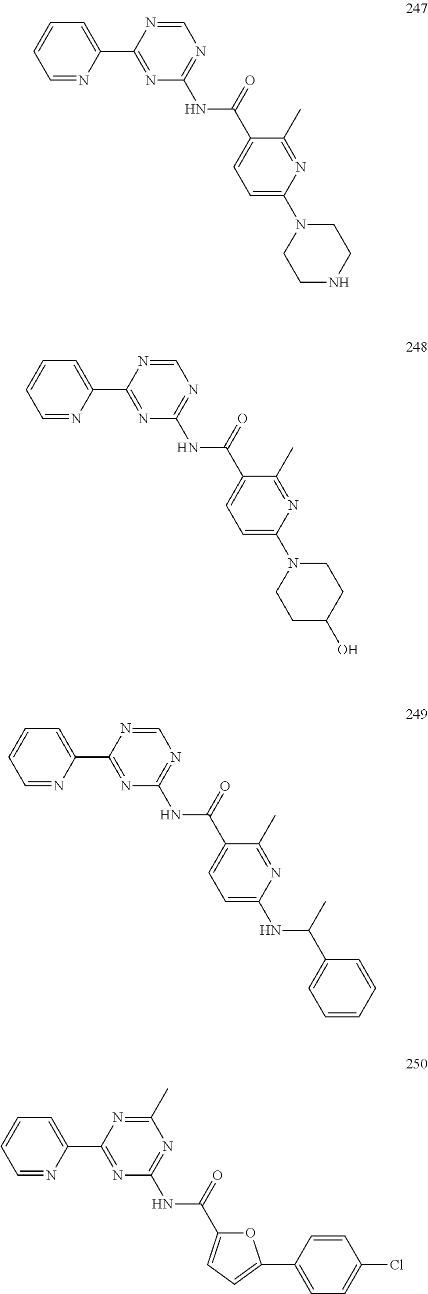

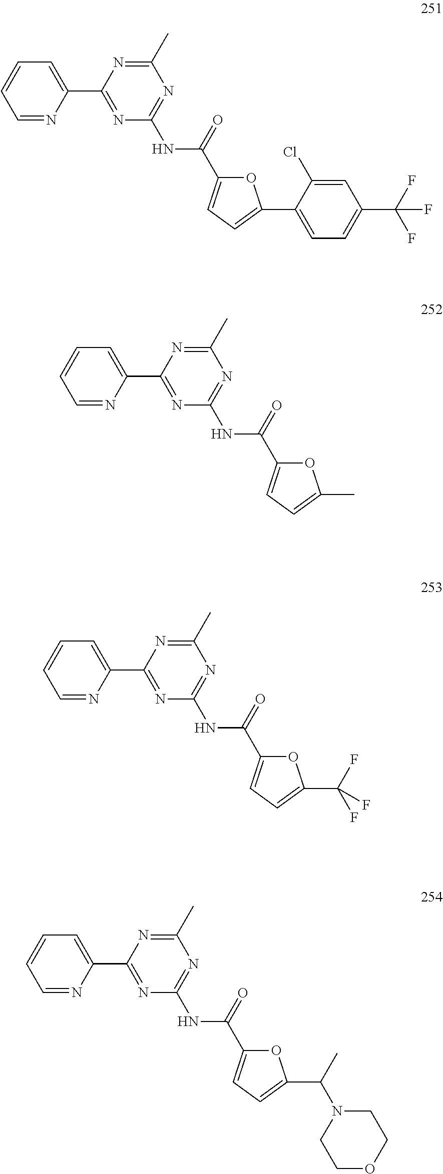

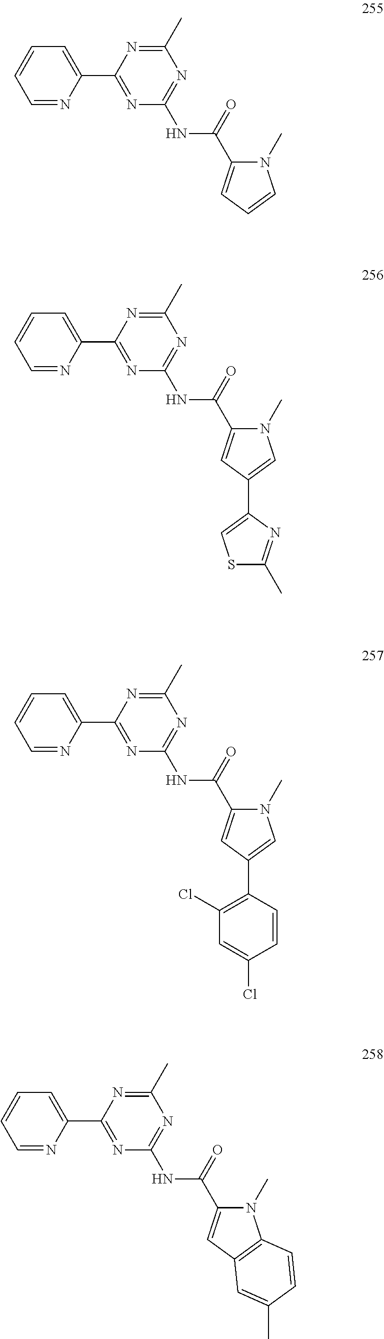

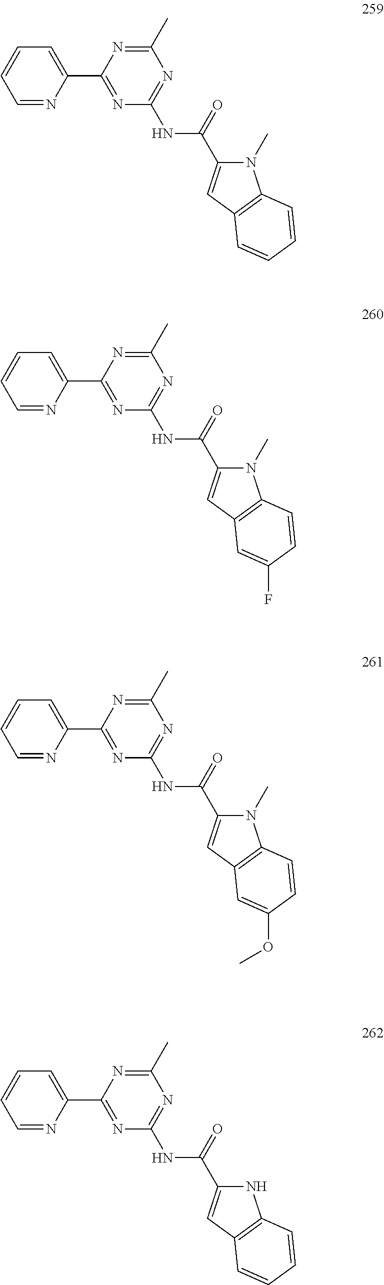

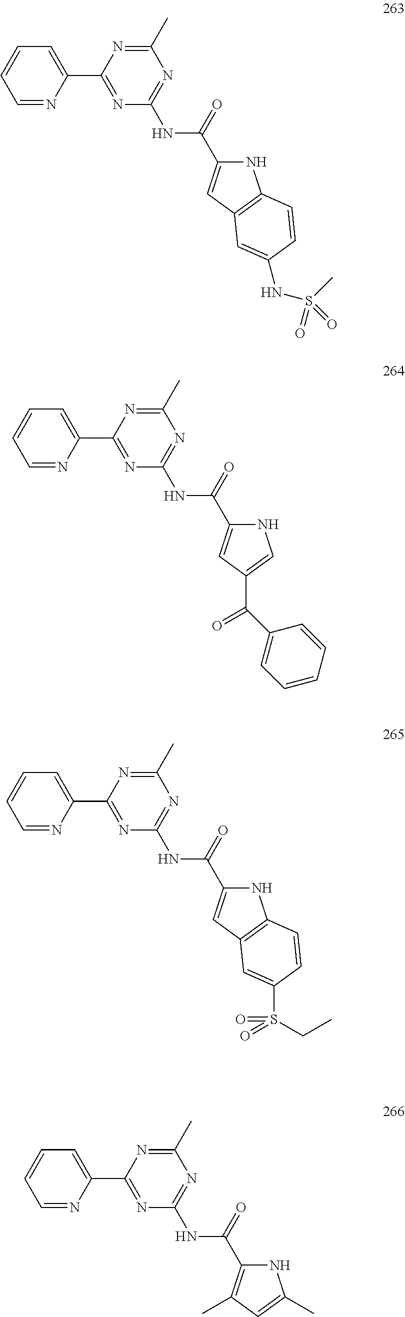

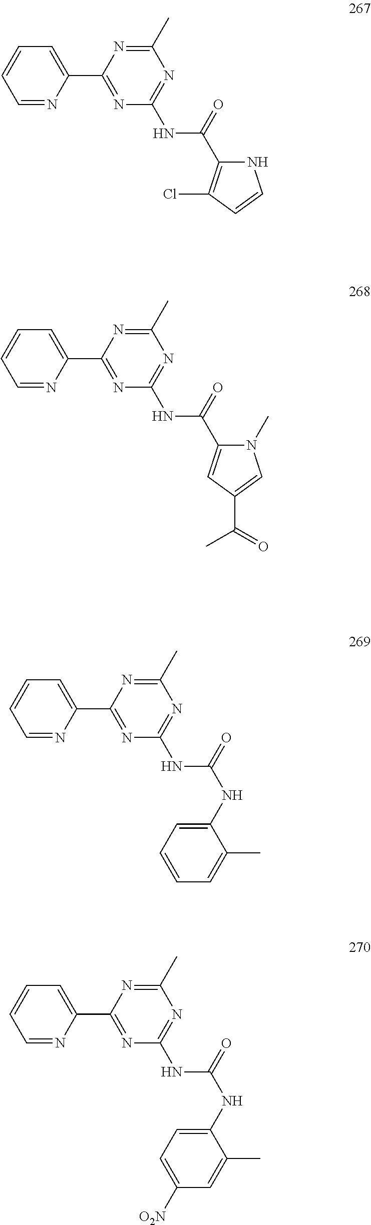

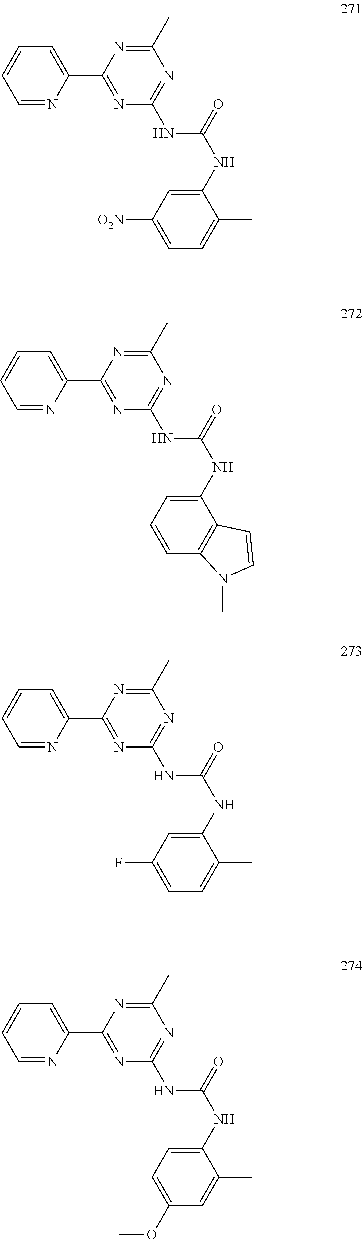

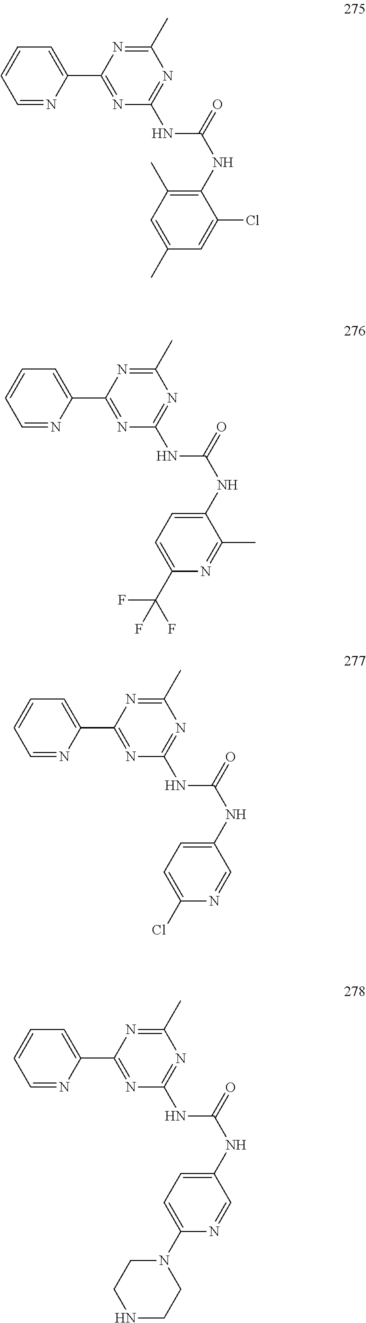

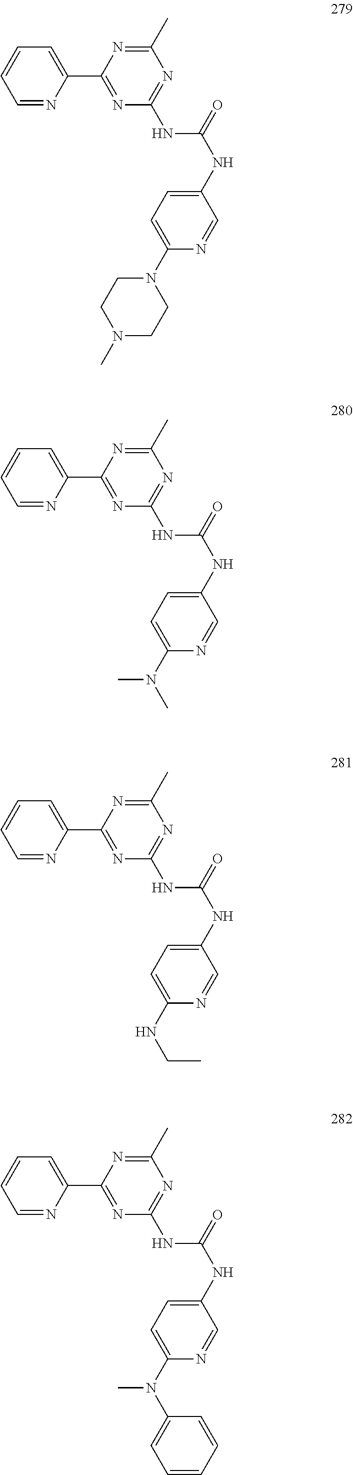

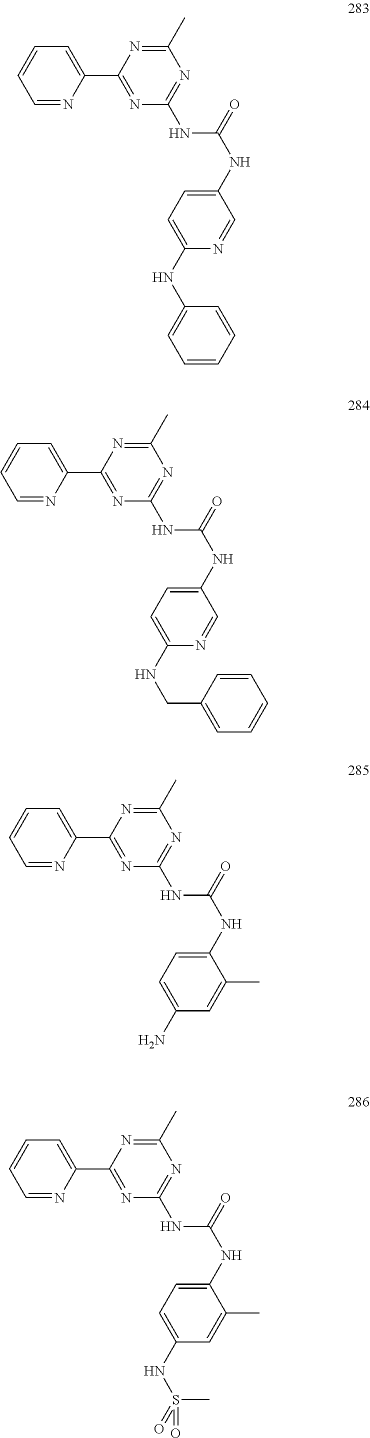

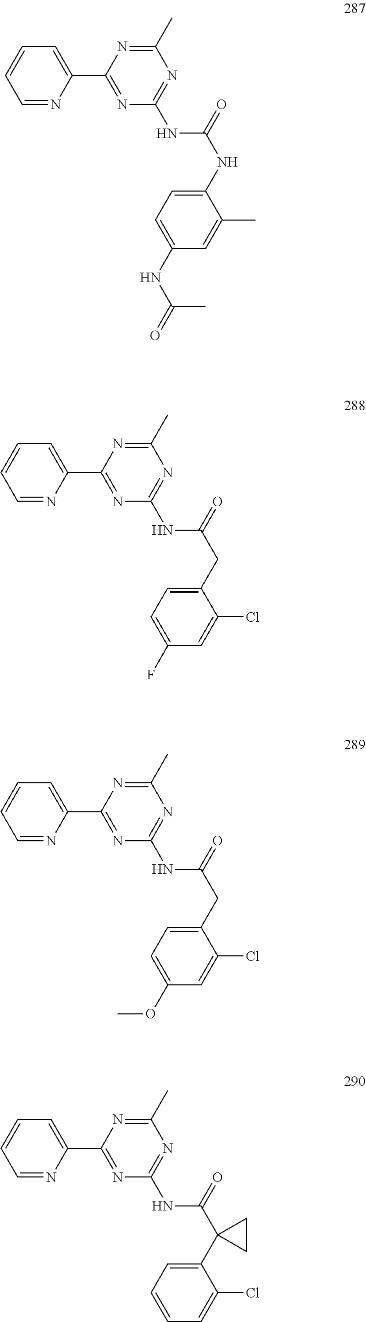

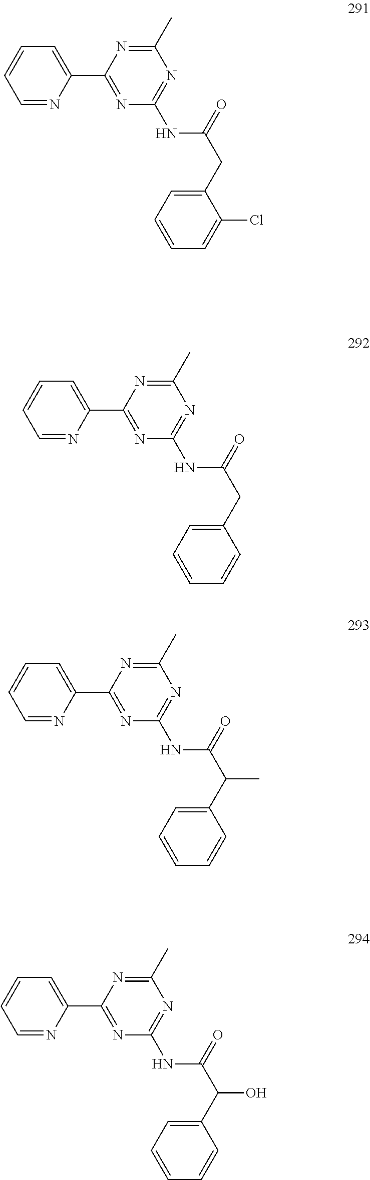

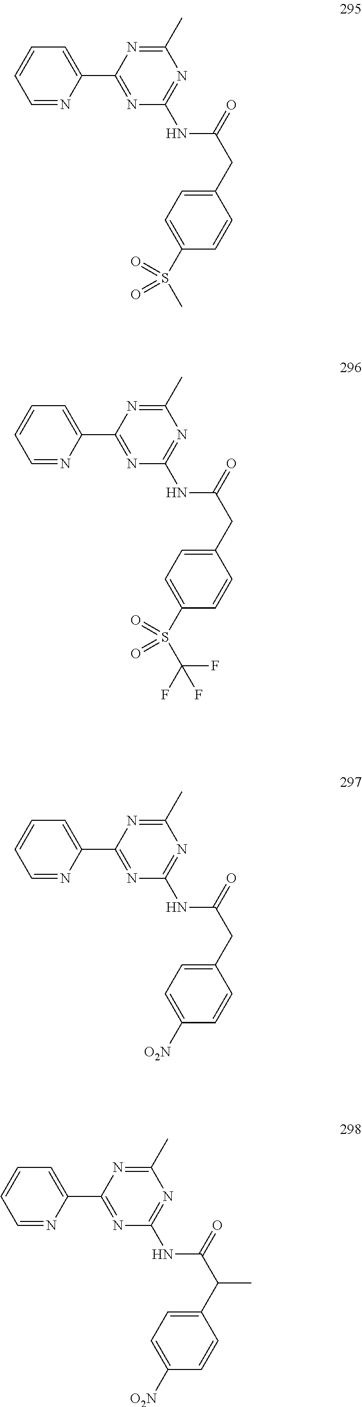

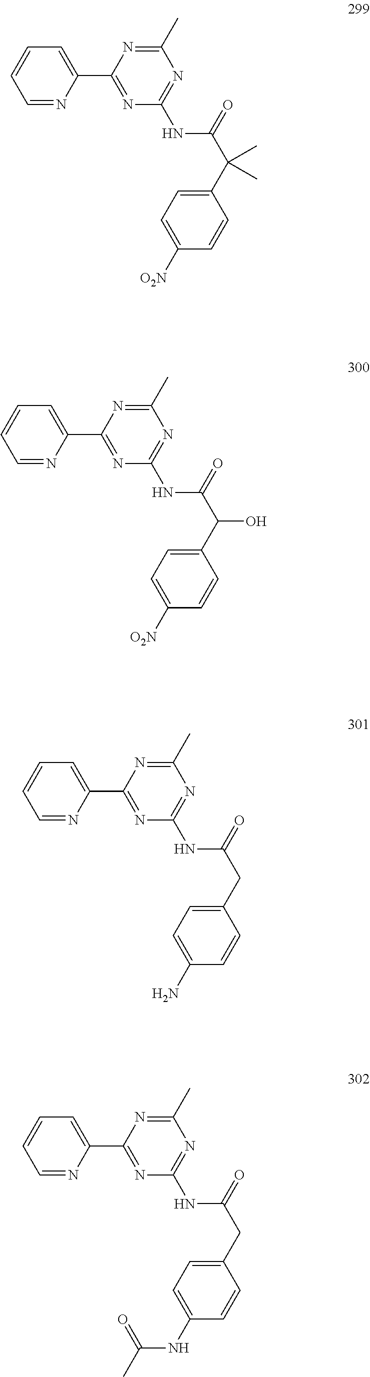

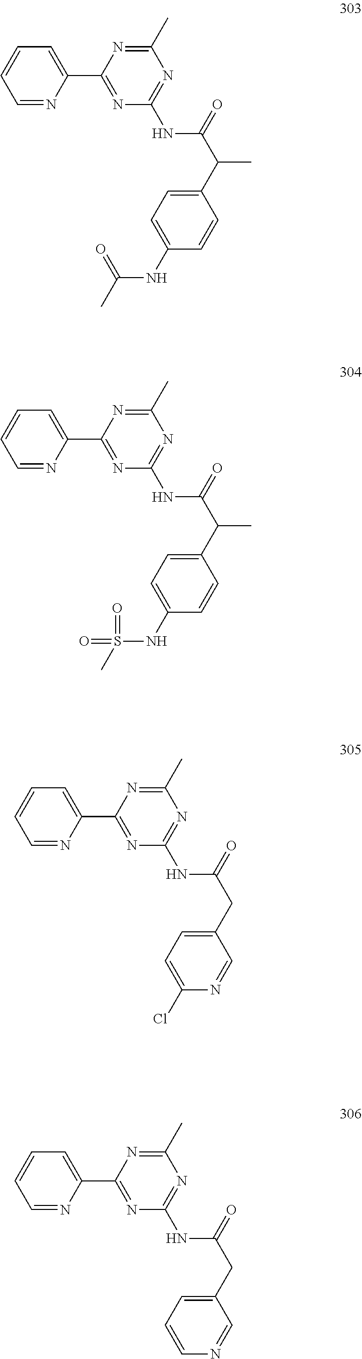

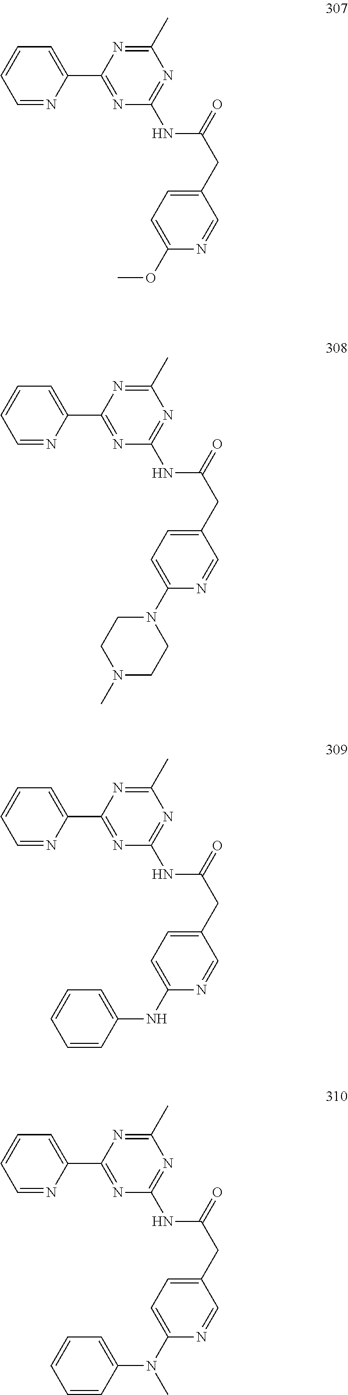

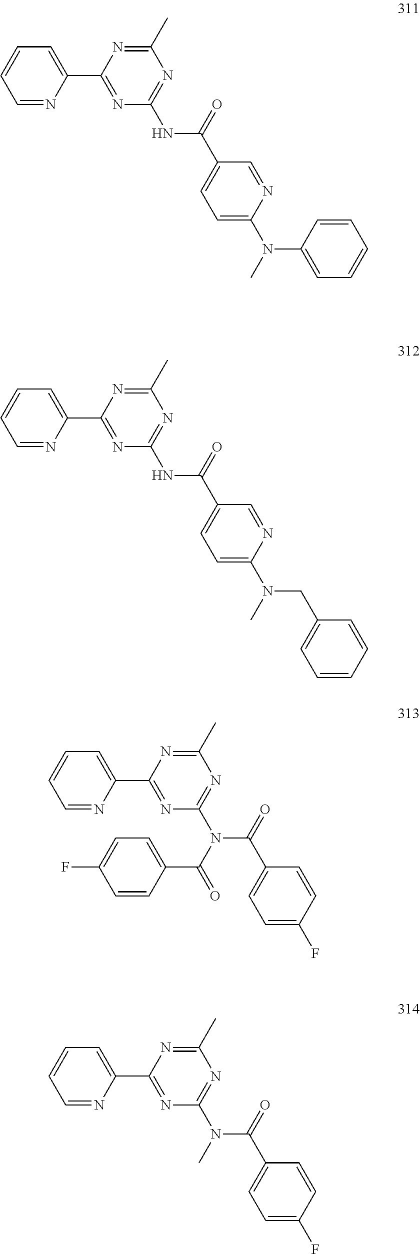

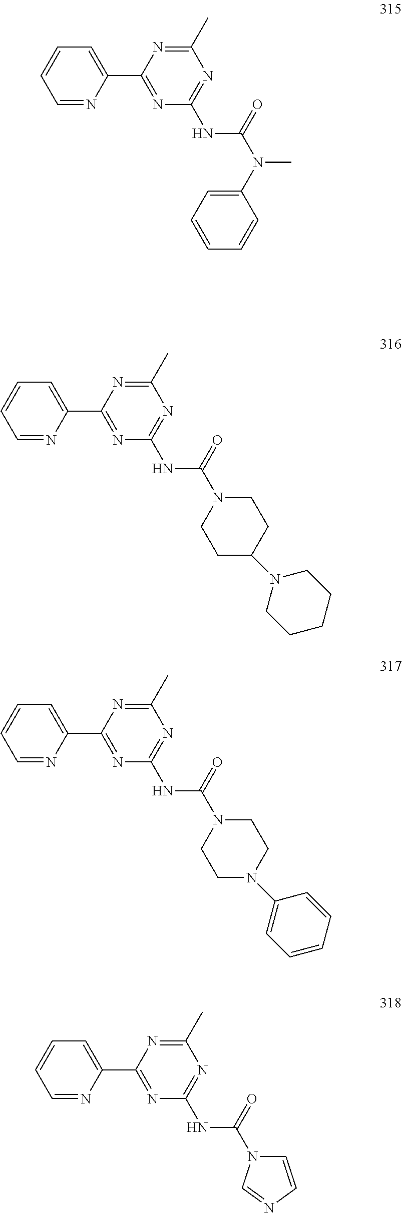

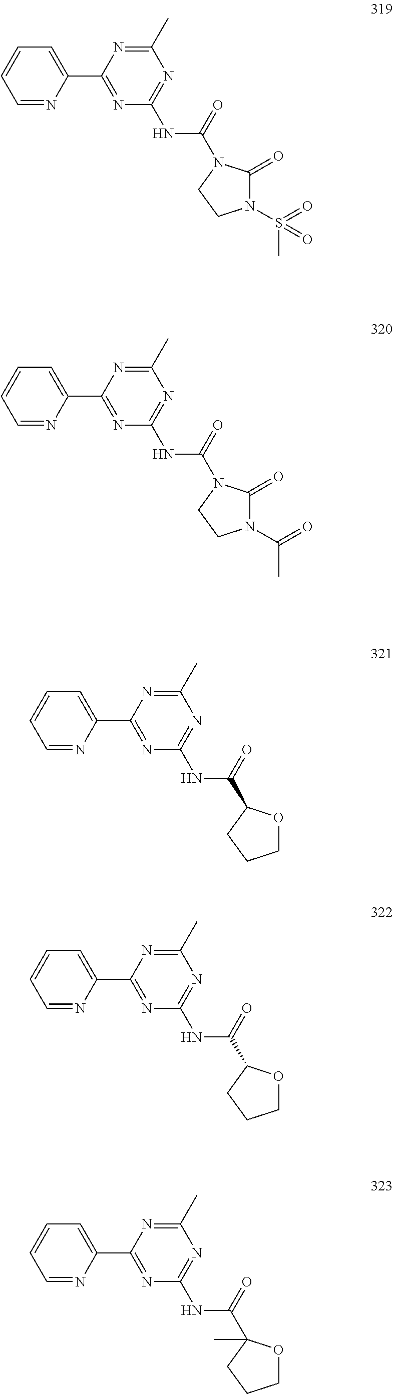

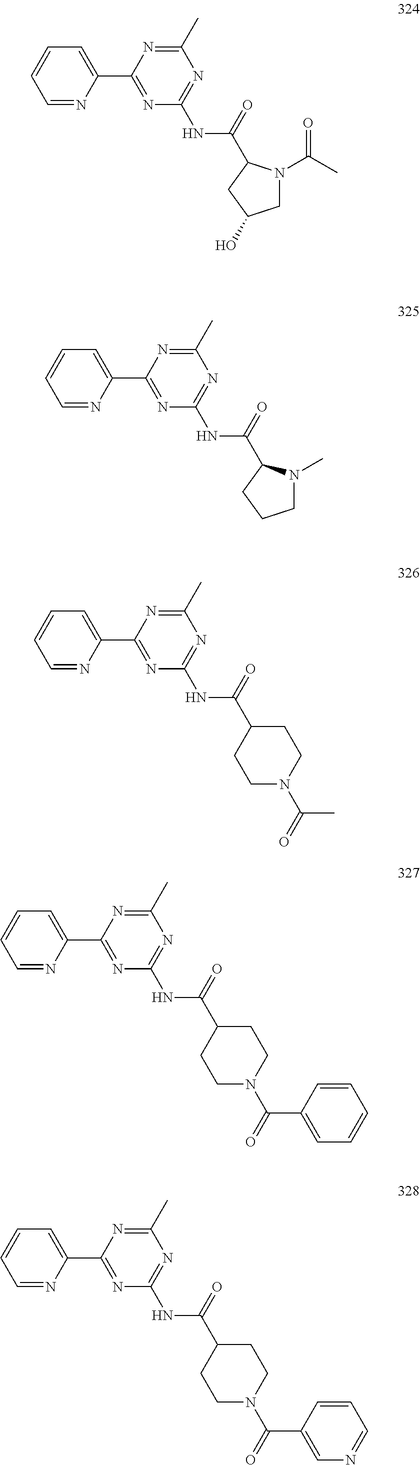

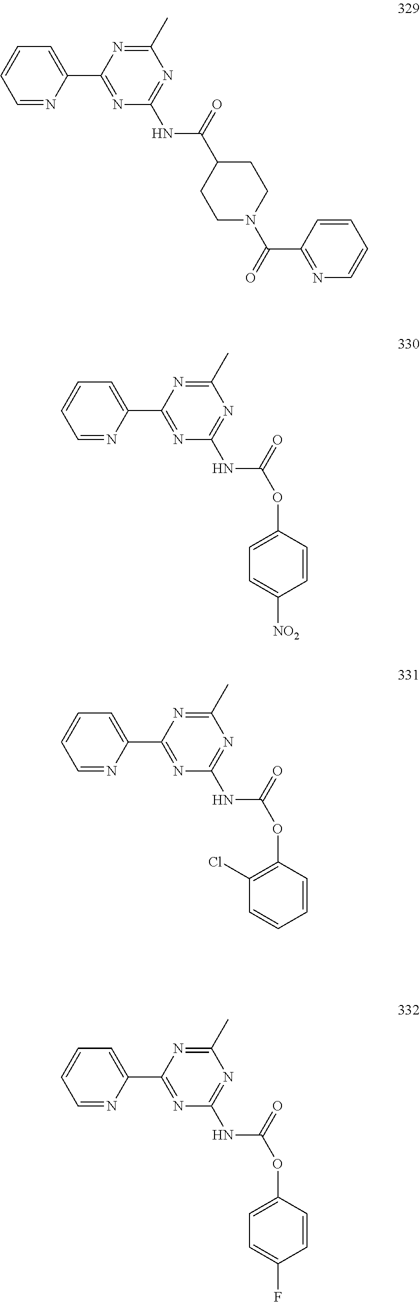

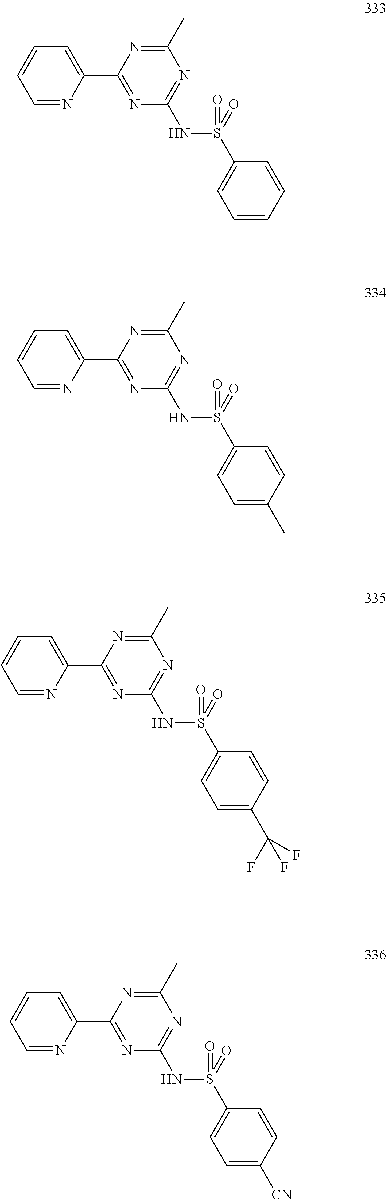

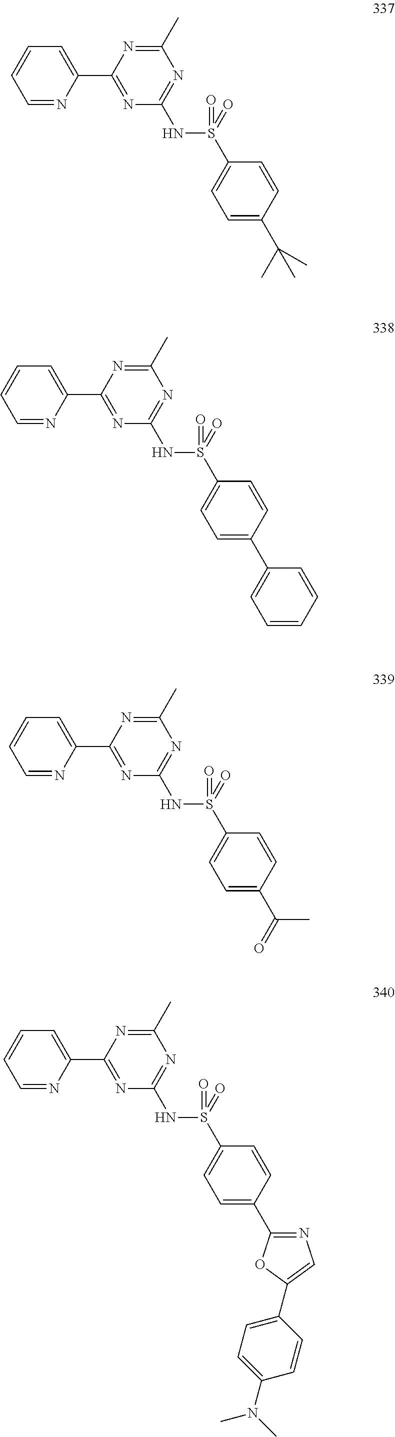

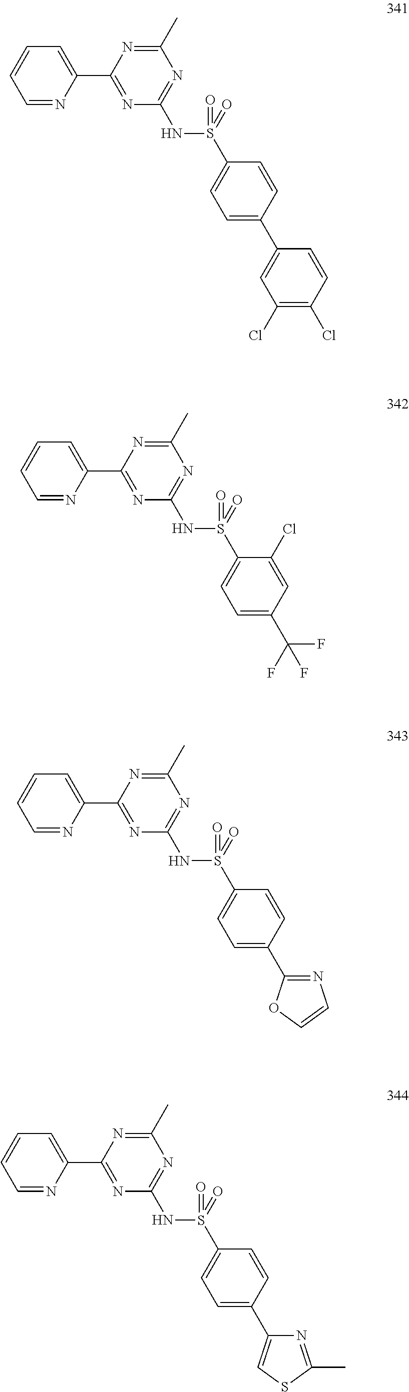

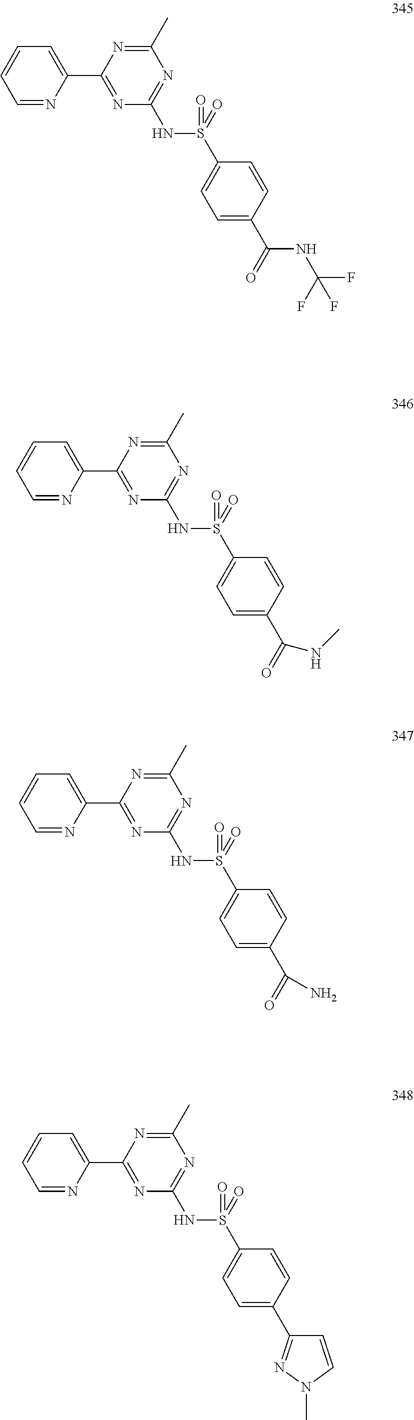

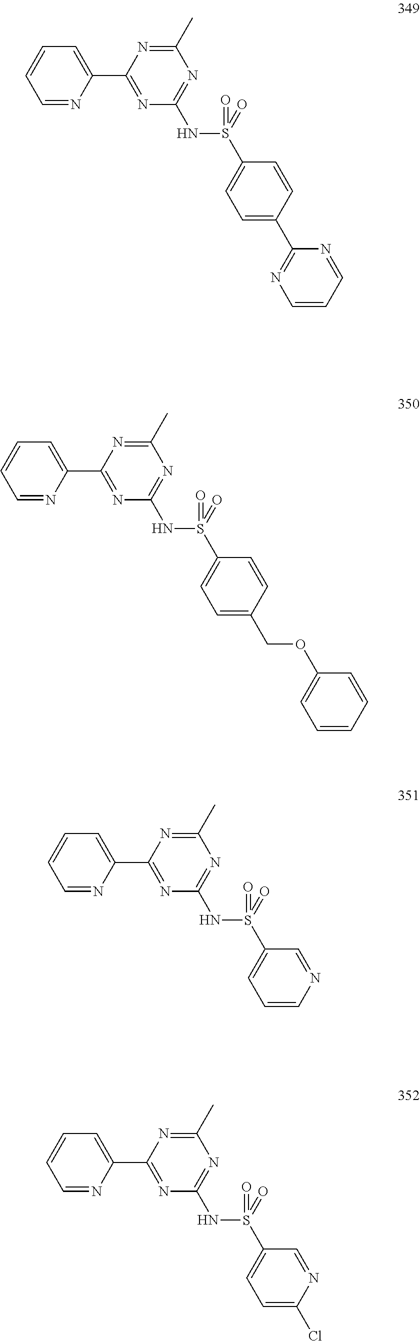

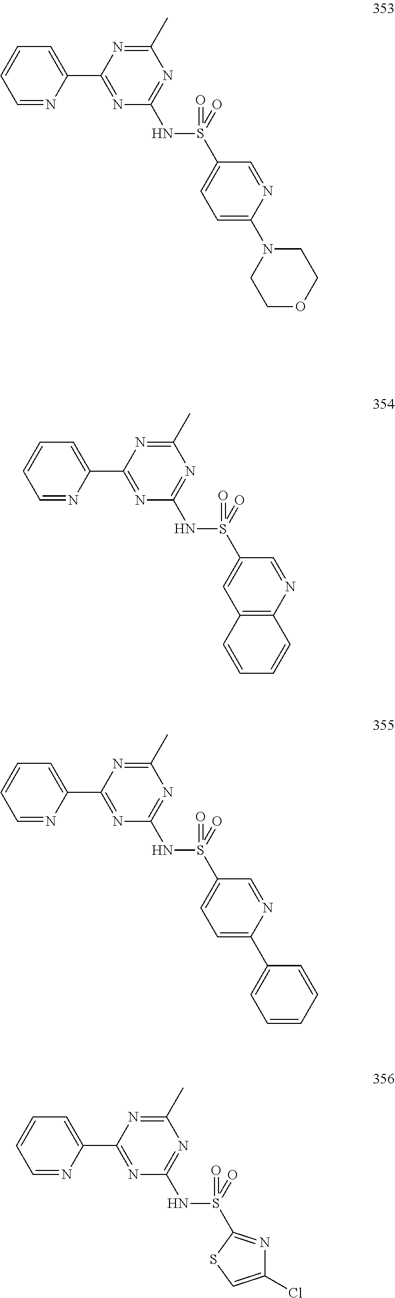

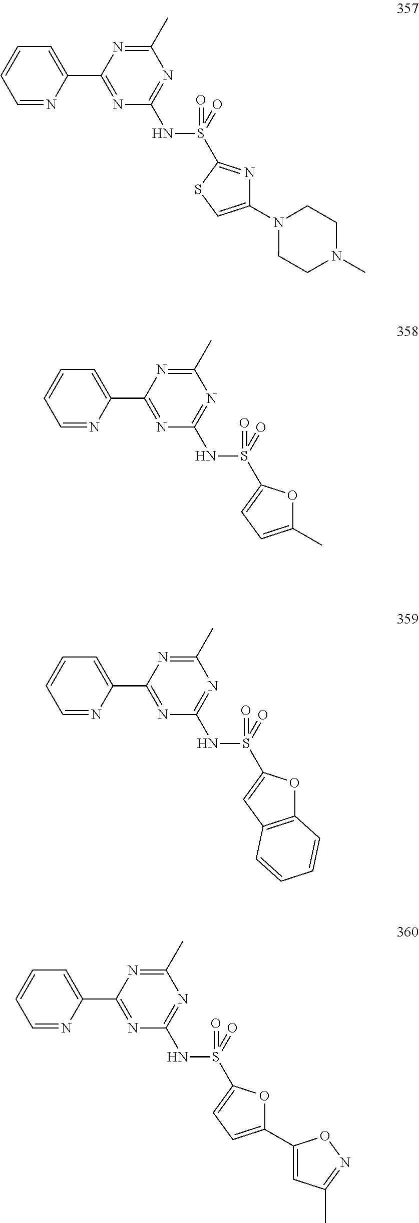

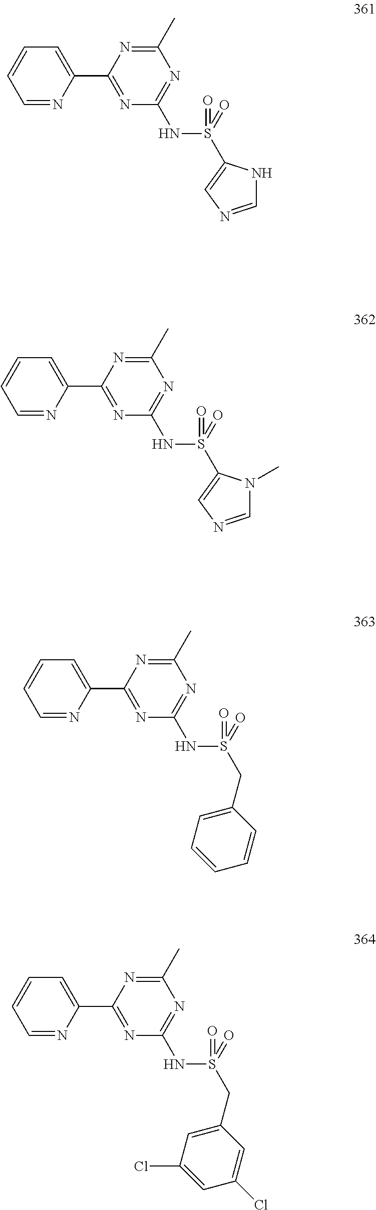

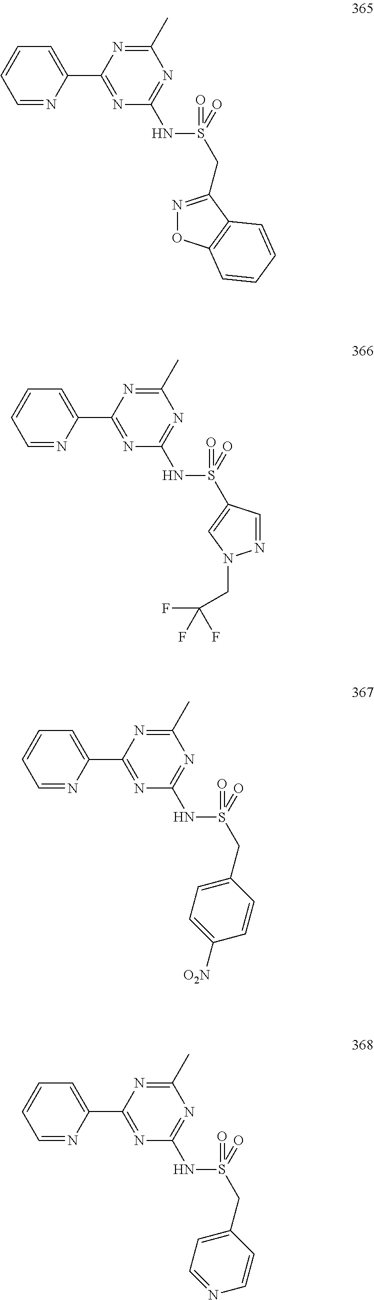

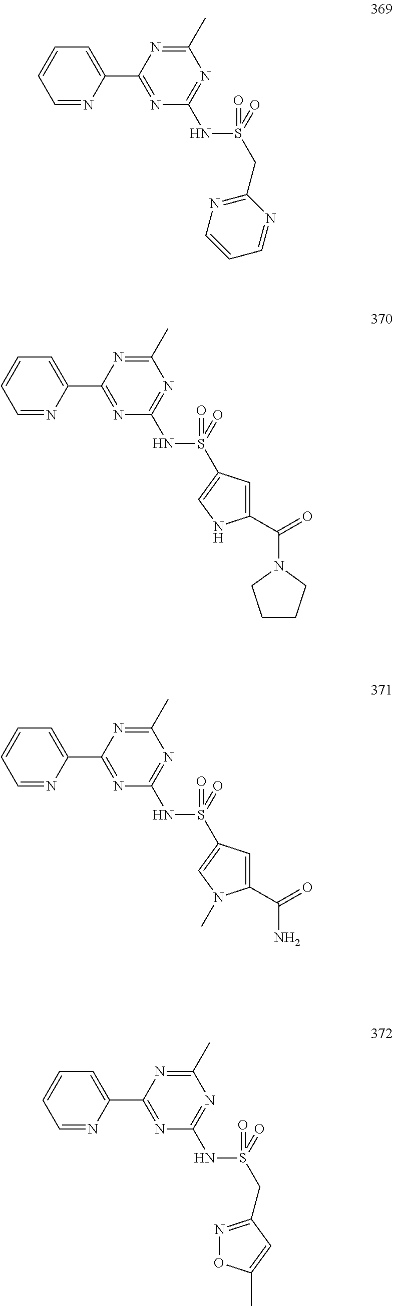

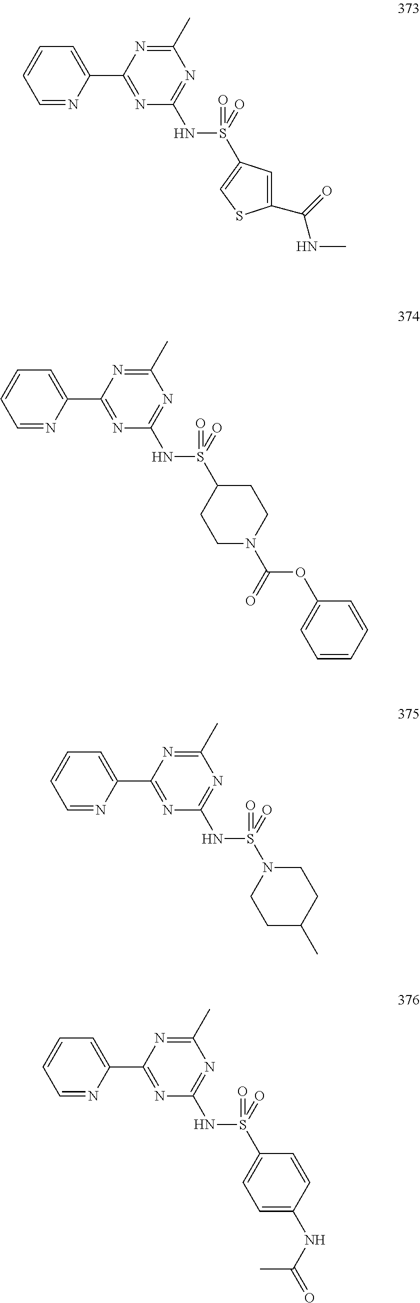

7. A compound as shown in Formula (A): 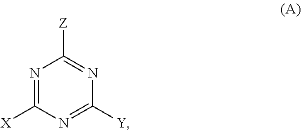
or a pharmaceutically acceptable salt thereof, wherein:
Y is selected from -K-A¹-R¹;
K is selected from NR³C(O) and NR⁴C(O)NR⁵;
A¹ is selected from aryl, heteroaryl, and heterocyclyl;
R₁ is one or more substituents independently selected from H, halo, nitro, C₁-C₆ alkylsulfonyl, —OR⁴, C₁-C₆ alkyl, and C₁-C₆ haloalkyl;
R³ is selected from H, C₁-C₆ alkyl, and —C(O)-A¹-R¹;
R⁴ and R⁵ are each independently selected from H and C₁-C₆ alkyl;
X is pyridinyl;
Z is selected from H, C₁-C₆ alkyl, C₁-C₆ alkylthio, C₁-C₆ alkoxy, C₁-C₆ haloalkyl, —NR⁴R⁵, and cyano.

8. A compound as shown in Formula (A): 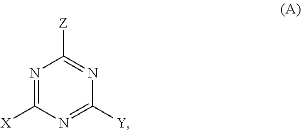
or a pharmaceutically acceptable salt thereof, wherein:
Y is -K-A¹-R¹;
K is selected from NR³C(O) and NR⁴C(O)NR⁵;
A¹ is selected from phenyl and furanyl;
R¹ is one or more substituents independently selected from H, halo, nitro, C₁-C₆ alkylsulfonyl, —OR⁴, C₁-C₆ alkyl, and C₁-C₆ haloalkyl;
R³ is selected from H, C₁-C₆ alkyl, and —C(O)-A¹-R¹;
R⁴ and R⁵ are each independently selected from H and C₁-C₆ alkyl;
X is pyridinyl;
Z is selected from C₁-C₆ alkyl, C₁-C₆ alkylthio, and —NR⁴R⁵.

9. A process for making compound of claim 7 or its pharmaceutically acceptable salts, hydrates, solvates, crystal forms salts and individual diastereomers thereof.

10. A pharmaceutical composition comprising at least one compound of claim 7 or its pharmaceutically acceptable salts, hydrates, solvates, crystal forms salts and individual diastereomers thereof, and a pharmaceutically acceptable carrier.

11. A process for making compound of claim 8 or its pharmaceutically acceptable salts, hydrates, solvates, crystal forms salts and individual diastereomers thereof.

12. A pharmaceutical composition comprising at least one compound of claim 8 or its pharmaceutically acceptable salts, hydrates, solvates, crystal forms salts and individual diastereomers thereof, and a pharmaceutically acceptable carrier.
